SEQUENCE LISTING (54 sequences)

SEQ ID NO: 1 - protein, 400 aa, Artificial Sequence
  Description of Artificial Sequence/note = synthetic construct
MDSSAAPTNASNCTDALAYSSCSPAPSPGSWVNLSHLDGNLSDPCGPNRTDLGGRDSLCPPTGSPSMITAITIMALYSIVCVVGLFGNFLVMYVIVRYTKMKTATNIYIFNLALADALATSTLPFQSVNYLMGTWPFGTILCKIVISIDYYNMFTSIFTLCTMSVDRYIAVCHPVKALDFRTPRNAKIINVCNWILSSAIGLPVMFMATTKYRQGSIDCTLTFSHPTWYWENLLKICVFIFAFIMPVLIITVCYGLMILRLKSVRMLSGSKEKDRNLRRITRMVLVVVAVFIVCWTPIHIYVIIKALVTIPETTFQTVSWHFCIALGYTNSCLNPVLYAFLDENFKRCFREFCIPTSSNIEQQNSTRIRQNTRDHPSTANTVDRTNHQLENLEAETAPLP

SEQ ID NO: 2 - DNA, 1200 bp, Artificial Sequence
  Description of Artificial Sequence/note = synthetic construct
atggacagcagcgctgcccccacgaacgccagcaattgcactgatgccttggcgtactcaagttgctccccagcacccagccccggttcctgggtcaacttgtcccacttagatggcaacctgtccgacccatgcggtccgaaccgcaccgacctgggcgggagagacagcctgtgccctccgaccggcagtccctccatgatcacggccatcacgatcatggccctctactccatcgtgtgcgtggtggggctcttcggaaacttcctggtcatgtatgtgattgtcagatacaccaagatgaagactgccaccaacatctacattttcaaccttgctctggcagatgccttagccaccagtaccctgcccttccagagtgtgaattacctaatgggaacatggccatttggaaccatcctttgcaagatagtgatctccatagattactataacatgttcaccagcatattcaccctctgcaccatgagtgttgatcgatacattgcagtctgccaccctgtcaaggccttagatttccgtactccccgaaatgccaaaattatcaatgtctgcaactggatcctctcttcagccattggtcttcctgtaatgttcatggctacaacaaaatacaggcaaggttccatagattgtacactaacattctctcatccaacctggtactgggaaaacctgctgaagatctgtgttttcatcttcgccttcattatgccagtgctcatcattaccgtgtgctatggactgatgatcttgcgcctcaagagtgtccgcatgctctctggctccaaagaaaaggacaggaatcttcgaaggatcaccaggatggtgctggtggtggtggctgtgttcatcgtctgctggactcccattcacatttacgtcatcattaaagccttggttacaatcccagaaactacgttccagactgtttcttggcacttctgcattgctctaggttacacaaacagctgcctcaacccagtcctttatgcatttctggatgaaaacttcaaacgatgcttcagagagttctgtatcccaacctcttccaacattgagcaacaaaactccactcgaattcgtcagaacactagagaccacccctccacggccaatacagtggatagaactaatcatcagctagaaaatctggaagcagaaactgctccgttgccc

SEQ ID NO: 3 - protein, 398 aa, Artificial Sequence
  Description of Artificial Sequence/note = synthetic construct
MDSSAGPGNISDCSDPLAPASWSPAPGSWLNLSHVDGNQSDPCGPNRTGLGGSHSLCPQTGSPSMVTAITIMALYSIVCVVGLFGNFLVMYVIVRYTKMKTATNIYIFNLALADALATSTLPFQSVNYLMGTWPFGNILCKIVISIDYYNMFTSIFTLCTMSVDRYIAVCHPVKALDFRTPRNAKIVNVCNWILSSAIGLPVMFMATTKYRQGSIDCTLTFSHPTWYWENLLKICVFIFAFIMPVLIITVCYGLMILRLKSVRMLSGSKEKDRNLRRITRMVLVVVAVFIVCWTPIHIYVIIKALITIPETTFQTVSWHFCIALGYTNSCLNPVLYAFLDENFKRCFREFCIPTSSTIEQQNSARIRQNTREHPSTANTVDRTNHQLENLEAETAPLP

SEQ ID NO: 4 - DNA, 1194 bp, Artificial Sequence
  Description of Artificial Sequence/note = synthetic construct
atggacagcagcgccggcccagggaacatcagcgactgctctgaccccttagctcctgcaagttggtccccagcacctggctcctggctcaacttgtcccacgttgatggcaaccagtccgacccatgcggtcctaaccgcacggggcttggcgggagccacagcctgtgccctcagaccggcagcccttccatggtcacagccatcaccatcatggccctctattctatcgtgtgtgtagtgggcctctttggaaacttcctggtcatgtatgtgattgtaagatataccaaaatgaagactgccaccaacatctacattttcaaccttgctctggcagatgccttagccactagcacgctgccctttcagagtgttaactacctgatgggaacgtggccctttggaaacatcctctgcaagatcgtgatctcaatagactactacaacatgttcaccagtatcttcaccctctgcaccatgagtgtagaccgctacattgccgtctgccacccggtcaaggccctggatttccgtaccccccgaaatgccaaaattgtcaatgtctgcaactggatcctctcttctgccattggtctgcccgtaatgttcatggcaaccacaaaatacaggcaggggtccatagattgcaccctcactttctctcatcccacatggtactgggagaacctgctcaaaatctgtgtcttcatcttcgccttcatcatgccggtcctcatcatcactgtgtgttatggactgatgatcttacgactcaagagtgtccgcatgctgtcgggctccaaagaaaaggacaggaacctgcgcaggatcacccggatggtgctggtggtcgtggctgtatttattgtctgctggacccccatccacatctatgtcatcatcaaagcactgatcacgattccagaaaccactttccagactgtttcctggcacttctgcattgccttgggttacacaaacagctgcctgaacccagttctttatgcgttcctggatgaaaacttcaaacgatgttttagagagttctgcatcccaacttcctccacaatcgaacagcaaaactctgctcgaatccgtcaaaacactagggaacacccctccacggctaatacagtggatcgaactaaccaccagctagaaaatctggaagcagaaactgctccattgccc

SEQ ID NO: 5 - DNA, 2423 bp, Artificial Sequence
  Description of Artificial Sequence/note = synthetic construct
acccgcgctcgtacgtgcgcctccgccggcagctcctgactcatcgggggctccgggtcacatgcgcccgcgcggccctataggcgcctcctccgcccgccgcccgggagccgcagccgccgccgccactgccactcccgctctctcagcgccgccgtcgccaccgccaccgccaccgccactaccaccgtctgagtctgcagtcccgagatcccagccatcatgtccatagagaagatctgggcccgggagatcctggactcccgcgggaaccccacagtggaggtggatctctatactgccaaaggtcttttccgggctgcagtgcccagtggagcctctacgggcatctatgaggccctggagctgagggatggagacaaacagcgttacttaggcaaaggtgtcctgaaggcagtggaccacatcaactccaccatcgcgccagccctcatcagctcaggtctctctgtggtggagcaagagaaactggacaacctgatgctggagttggatgggactgagaacaaatccaagtttggggccaatgccatcctgggtgtgtctctggccgtgtgtaaggcaggggcagctgagcgggaactgcccctgtatcgccacattgctcagctggccgggaactcagacctcatcctgcctgtgccggccttcaacgtgatcaatggtggctctcatgctggcaacaagctggccatgcaggagttcatgatcctcccagtgggagctgagagctttcgggatgccatgcgactaggtgcagaggtctaccatacactcaagggagtcatcaaggacaaatacggcaaggatgccaccaatgtgggggatgaaggtggctttgcccccaatatcctggagaacagtgaagccttggagctggtgaaggaagccatcgacaaggctggctacacggaaaagatcgttattggcatggatgttgctgcctcagagttttatcgtgatggcaaatatgacttggacttcaagtctcccactgatccttcccgatacatcactggggaccagctgggggcactctaccaggactttgtcagggactatcctgtggtctccattgaggacccatttgaccaggatgattgggctgcctggtccaagttcacagccaatgtagggatccagattgtgggtgatgacctgacagtgaccaacccaaaacgtattgagcgggcagtggaagaaaaggcctgcaactgtctgctgctcaaggtcaaccagatcggctcggtcactgaagccatccaagcgtgcaagctggcccaggagaatggctggggggtcatggtgagtcatcgctcaggagagactgaggacacattcattgctgacctggtggtggggctgtgcacaggccagatcaagactggtgccccgtgccgttctgaacgtctggctaaatacaaccagctcatgagaattgaggaagagctgggggatgaagctcgctttgccggacataacttccgtaatcccagtgtgctgtgattcctctgcttgcctggagacgtggaacctctgtctcatcctcctggaaccttgctgtcctgatctgtgatagttcaccccctgagatcccctgagccccagggtgcccagaacttccctgattgacctgctccgctgctccttggcttacctgacctcttgctgtctctgctcgccctcctttctgtgccctactcattggggttccgcactttccacttcttcctttctctttctctcttccctcagaaactagaaatgtgaatgaggattattataaaagggggtccgtggaagaatgatcagcatctgtgatgggagcgtcagggttggtgtgctgaggtgttagagagggaccatgtgtcacttgtgctttgctcttgtcccacgtgtcttccactttgcatatgagccgtgaactgtgcatagtgctgggatggaggggagtgttgggcatgtgatcacgcctggctaataaggctttagtgtatttatttatttatttattttatttgtttttcattcatcccattaatcatttccccataactcaatggcctaaaactggcctgacttgggggaacgatgtgtctgtatttcatgtggctgtagatcccaagatgactggggtgggaggtcttgctagaatgggaagggtcatagaaagggccttgacatcagttcctttgtgtgtactcactgaagcctgcgttggtccagagcggaggctgtgtgcctgggggagttttcctctatacatctctccccaaccctaggttccctgttcttcctccagctgcaccagagcaacctctcactccccatgccacgttccacagttgccaccacctctgtggcattgaaatgagcacctccattaaagtctgaatcagtgc

SEQ ID NO: 6 - DNA, 1773 bp, Artificial Sequence
  Description of Artificial Sequence/note = synthetic construct
ccgaggagcctgcgctgctcctggctcacagcgctccgggcgaggagagcgggcggaccggggggctgggccggtgcgggcggcgaggcaggcggacgaggcgcagagacagcggggcggccggggcgcggcacgcggcgggtcggggccggcctctgccttgccgctcccctcgcgtcggatccccgcgcccaggcagccggtggagagggacgcggcggacgccggcagccatggaaccggccccctccgccggcgccgagctgcagcccccgctcttcgccaacgcctcggacgcctaccctagcgccttccccagcgctggcgccaatgcgtcggggccgccaggcgcgcggagcgcctcgtccctcgccctggcaatcgccatcaccgcgctctactcggccgtgtgcgccgtggggctgctgggcaacgtgcttgtcatgttcggcatcgtccggtacactaagatgaagacggccaccaacatctacatcttcaacctggccttagccgatgcgctggccaccagcacgctgcctttccagagtgccaagtacctgatggagacgtggcccttcggcgagctgctctgcaaggctgtgctctccatcgactactacaatatgttcaccagcatcttcacgctcaccatgatgagtgttgaccgctacatcgctgtctgccaccctgtcaaggccctggacttccgcacgcctgccaaggccaagctgatcaacatctgtatctgggtcctggcctcaggcgttggcgtgcccatcatggtcatggctgtgacccgtccccgggacggggcagtggtgtgcatgctccagttccccagccccagctggtactgggacacggtgaccaagatctgcgtgttcctcttcgccttcgtggtgcccatcctcatcatcaccgtgtgctatggcctcatgctgctgcgcctgcgcagtgtgcgcctgctgtcgggctccaaggagaaggaccgcagcctgcggcgcatcacgcgcatggtgctggtggttgtgggcgccttcgtggtgtgttgggcgcccatccacatcttcgtcatcgtctggacgctggtggacatcgaccggcgcgacccgctggtggtggctgcgctgcacctgtgcatcgcgctgggctacgccaatagcagcctcaaccccgtgctctacgctttcctcgacgagaacttcaagcgctgcttccgccagctctgccgcaagccctgcggccgcccagaccccagcagcttcagccgcgcccgcgaagccacggcccgcgagcgtgtcaccgcctgcaccccgtccgatggtcccggcggtggcgctgccgcctgaccaggccatccggcccccagacgcccctccctagttgtacccggaggccacatgagtcccagtgggaggcgcgagccatgatgtggagtggggccagtagataggtcggagggctttgggaccgccagatggggcctctgtttcggagacgggaccgggccgctagatgggcatggggtgggcctctggtttggggcgaggcagaggacagatcaatggcgcagtgcctctggtctgggtgcccccgtccacggctctaggtggggcgggaaagccagtgactccaggagaggagcgggacctgtggctctacaactgagtccttaaacagggcatctccaggaaggcggggcttcaaccttgagacagcttcggtttctaacttggagccggactttcggagttggggggtccggggccc

SEQ ID NO: 7 - DNA, 9426 bp, Artificial Sequence
  Description of Artificial Sequence/note = synthetic construct
atgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctgtgaaacttcgaggagtctctttgttgaggacttttgagttctcccttgaggctcccacagatacaataaatatttgagattgaaccctgtcgagtatctgtgtaatcttttttacctgtgaggtctcggaatccgggccgagaacttcgcagttggcgcccgaacagggacttgattgagagtgattgaggaagtgaagctagagcaatagaaagctgttaagcagaactcctgctgacctaaatagggaagcagtagcagacgctgctaacagtgagtatctctagtgaagcggactcgagctcataatcaagtcattgtttaaaggcccagataaattacatctggtgactcttcgcggaccttcaagccaggagattcgccgagggacagtcaacaaggtaggagagattctacagcaacatggggaatggacaggggcgagattggaaaatggccattaagagatgtagtaatgttgctgtaggagtaggggggaagagtaaaaaatttggagaagggaatttcagatgggccattagaatggctaatgtatctacaggacgagaacctggtgatataccagagactttagatcaactaaggttggttatttgcgatttacaagaaagaagagaaaaatttggatctagcaaagaaattgatatggcaattgtgacattaaaagtctttgcggtagcaggacttttaaatatgacgggtgtctactgctgctgcagctgaaaatatgtattctcaaatgggattagacactaggccatctatgaaagaagcaggtggaaaagaggaaggccctccacaggcatatcctattcaaacagtaaatggagtaccacaatatgtagcacttgacccaaaaatggtgtccatttttatggaaaaggcaagagaaggactaggaggtgaggaagttcaactatggtttactgccttctctgcaaatttaacacctactgacatggccacattaataatggccgcaccagggtgcgctgcagataaagaaatattggatgaaagcttaaagcaactgacagcagaatatgatcgcacacatccccctgatgctcccagaccattaccctattttactgcagcagaaattatgggtataggattaactcaagaacaacaagcagaagcaagatttgcaccagctaggatgcagtgtagagcatggtatctcgaggcattaggaaaattggctgccataaaagctaagtctcctcgagctgtgcagttaagacaaggagctaaggaagattattcatcctttatagacagattgtttgcccaaatagatcaagaacaaaatacagctgaagttaagttatatttaaaacagtcattgagcatagctaatgctaatgcagactgtaaaaaggcaatgagccaccttaagccagaaagtaccctagaagaaaagttgagagcttgtcaagaaataggctcaccaggatataaaatgcaactcttggcagaagctcttacaaaagttcaagtagtgcaatcaaaaggatcaggaccagtgtgttttaattgtaaaaaaccaggacatctagcaagacaatgtagagaagtgaaaaaatgtaataaatgtggaaaacctggtcatgtagctgccaaatgttggcaaggaaatagaaagaattgtacaagggaagaaagggatacaacaattacaaaagtgggaagattgggtaggatggataggaaatattccacaatatttaaagggactattgggaggtatcttgggaataggattaggagtgttattattgattttatgtttacctacattggttgattgtataagaaattgtatccacaagatactaggatacacagtaattgcaatgcctgaagtagaaggagaagaaatacaaccacaaatggaattgaggagaaatggtaggcaatgtggcatgtctgaaaaagaggaggaatgatgaagtatctcagacttattttataagggagatactgtgctgagttcttccctttgaggaaggtatgtcatatgaatccatttcgaaatcaaattcctcctctgctcgcccaatccttccaaccccctatggtggtatggctgacacagaaaatgtctgctcctgtatgggacatttgcccctcttctccaaatataagacaggatgaggcctagcttttgctgctccaaagttttaaaagaacacattgcacggcatttagggactctaaagggtggaggaggaatgagggaattgcatcatgccaaggctggtcctcatccatcactgcttccagggcccagagtggcttccaggaggtattcttacaaaggaagcccgatctgtagctaacactcagagcccattttcctgcgttaacccctcccgacctcatatacaggagtaacatgatcagtgacctgggggagctggccaaactgcgggacctgcccaagctgagggccttggtgctgctggacaacccctgtgccgatgagactgactaccgccaggaggccctggtgcagatggcacacctagagcgcctagacaaagagtactatgaggacgaggaccgggcagaagctgaggagatccgacagaggctgaaggaggaacaggagcaagaactcgacccggaccaagacatggaaccgtacctcccgccaacttagtggctcctctagcctgcagggacagtaaaggtgatggcaggaaggcagcccccggaggtcaaaggctgggcacgcgggaggagaggccagagtcagaggctgcgggtatctcagatatgaaggaaagatgagagaggctcaggaagaggtaagaaaagacacaagagaccagagaagggagaagaattagagagggaggcagaggaccgctgtctctacagacatagctggtagagactgggaggaagggatgaaccctgagcgcatgaagggaaggaggtggctggtggtatatggaggatgtagctgggccagggaaaagatcctgcactaaaaatctgaagctaaaaataacaggacacggggtggagaggcgaaaggagggcagattgaggcagagagactgagaggcctggggatgtgggcattccggtagggcacacagttcacttgtcttctctttttccaggaggccaragatgctgacctcaagaactcataataccccagtggggaccaccgcattcatagccctgttacaagaagtgggagatgttcctttttgtcccagactggaaatccattacatcccgaggctcaggttctgtggtggtcatctctgtgtggcttgttctgtgggcctacctaaagtcctaagcacagctctcaagcagatccgaggcgactaagatgctagtaggggttgtctggagagaagagccgaggaggtgggctgtgatggatcagttcagctttcaaataaaaaggcgtttttatattctgtgtcgagttcgtgaacccctgtggtgggcttctccatctgtctgggttagtacctgccactatactggaataaggggacgcctgcttccctcgagttggctggacaaggttatgagcatccgtgtacttatggggttgccagcttggtcctggatcgcccgggcccttcccccacccgttcggttccccaccaccacccgcgctcgtacgtgcgtctccgcctgcagctcttgactcatcggggcccccgggtcacatgcgctcgctcggctctataggcgccgccccctgcccaccccccgcccgcgctgggagccgcagccgccgccactcctgctctctctgcgccgccgccgtcaccaccgccaccgccaccggctgagtctgcagtcctcgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggccaagctaagcttgatatcgaattccggatgagcctctgtgaactactaaggtgggagggggctatacgcagaggagaatgtcagatgctcagctcggtcccctccgcctgacgctcctctctgtctcagccaggactggtttctgtaagaaacagcaggagctgtggcagcggcgaaaggaagcggctgaggcgcttggaacccgaaaagtctcggtgctcctggctacctcgcacagcggtgcccgcccggccgtcagtaccatggacagcagcgctgcccccacgaacgccagcaattgcactgatgccttggcgtactcaagttgctccccagcacccagccccggttcctgggtcaacttgtcccacttagatggcaacctgtccgacccatgcggtccgaaccgcaccgacctgggcgggagagacagcctgtgccctccgaccggcagtccctccatgatcacggccatcacgatcatggccctctactccatcgtgtgcgtggtggggctcttcggaaacttcctggtcatgtatgtgattgtcagatacaccaagatgaagactgccaccaacatctacattttcaaccttgctctggcagatgccttagccaccagtaccctgcccttccagagtgtgaattacctaatgggaacatggccatttggaaccatcctttgcaagatagtgatctccatagattactataacatgttcaccagcatattcaccctctgcaccatgagtgttgatcgatacattgcagtctgccaccctgtcaaggccttagatttccgtactccccgaaatgccaaaattatcaatgtctgcaactggatcctctcttcagccattggtcttcctgtaatgttcatggctacaacaaaatacaggcaaggttccatagattgtacactaacattctctcatccaacctggtactgggaaaacctgctgaagatctgtgttttcatcttcgccttcattatgccagtgctcatcattaccgtgtgctatggactgatgatcttgcgcctcaagagtgtccgcatgctctctggctccaaagaaaaggacaggaatcttcgaaggatcaccaggatggtgctggtggtggtggctgtgttcatcgtctgctggactcccattcacatttacgtcatcattaaagccttggttacaatcccagaaactacgttccagactgtttcttggcacttctgcattgctctaggttacacaaacagctgcctcaacccagtcctttatgcatttctggatgaaaacttcaaacgatgcttcagagagttctgtatcccaacctcttccaacattgagcaacaaaactccactcgaattcgtcagaacatctagagaccacccctccacggccaatacagtggatagaactaatcatcagctagaaaatctggaagcagaaactgctccgttgccctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggtaaaaaagaaaaaagggtggactgggatgagtattggaaccctgaagaaatagaaagaatgcttatggactagggactgtttacgaacaaatgataaaaggaaatagctgagcatgactcatagttaaagcgctagcagctgcctaaccgcaaaaccacatcctatggaaagcttgctaatgacgtataagttgttccattgtaagagtatataaccagtgctttgtgaaacttcgaggagtctctttgttgaggacttttgagttctcccttgaggctcccacagatacaataaatatttgagattgaaccctgtcgagtatctgtgtaatcttttttacctgtgaggtctcggaatccgggccgagaacttcgcagcggccgctcatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctggatcccgtcgacctcgagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtg

SEQ ID NO: 8 - DNA, 12745 bp, Artificial Sequence
  Description of Artificial Sequence/note = synthetic construct
atgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctgtgaaacttcgaggagtctctttgttgaggacttttgagttctcccttgaggctcccacagatacaataaatatttgagattgaaccctgtcgagtatctgtgtaatcttttttacctgtgaggtctcggaatccgggccgagaacttcgcagttggcgcccgaacagggacttgattgagagtgattgaggaagtgaagctagagcaatagaaagctgttaagcagaactcctgctgacctaaatagggaagcagtagcagacgctgctaacagtgagtatctctagtgaagcggactcgagctcataatcaagtcattgtttaaaggcccagataaattacatctggtgactcttcgcggaccttcaagccaggagattcgccgagggacagtcaacaaggtaggagagattctacagcaacatggggaatggacaggggcgagattggaaaatggccattaagagatgtagtaatgttgctgtaggagtaggggggaagagtaaaaaatttggagaagggaatttcagatgggccattagaatggctaatgtatctacaggacgagaacctggtgatataccagagactttagatcaactaaggttggttatttgcgatttacaagaaagaagagaaaaatttggatctagcaaagaaattgatatggcaattgtgacattaaaagtctttgcggtagcaggacttttaaatatgacgggtgtctactgctgctgcagctgaaaatatgtattctcaaatgggattagacactaggccatctatgaaagaagcaggtggaaaagaggaaggccctccacaggcatatcctattcaaacagtaaatggagtaccacaatatgtagcacttgacccaaaaatggtgtccatttttatggaaaaggcaagagaaggactaggaggtgaggaagttcaactatggtttactgccttctctgcaaatttaacacctactgacatggccacattaataatggccgcaccagggtgcgctgcagataaagaaatattggatgaaagcttaaagcaactgacagcagaatatgatcgcacacatccccctgatgctcccagaccattaccctattttactgcagcagaaattatgggtataggattaactcaagaacaacaagcagaagcaagatttgcaccagctaggatgcagtgtagagcatggtatctcgaggcattaggaaaattggctgccataaaagctaagtctcctcgagctgtgcagttaagacaaggagctaaggaagattattcatcctttatagacagattgtttgcccaaatagatcaagaacaaaatacagctgaagttaagttatatttaaaacagtcattgagcatagctaatgctaatgcagactgtaaaaaggcaatgagccaccttaagccagaaagtaccctagaagaaaagttgagagcttgtcaagaaataggctcaccaggatataaaatgcaactcttggcagaagctcttacaaaagttcaagtagtgcaatcaaaaggatcaggaccagtgtgttttaattgtaaaaaaccaggacatctagcaagacaatgtagagaagtgaaaaaatgtaataaatgtggaaaacctggtcatgtagctgccaaatgttggcaaggaaatagaaagaattgtacaagggaagaaagggatacaacaattacaaaagtgggaagattgggtaggatggataggaaatattccacaatatttaaagggactattgggaggtatcttgggaataggattaggagtgttattattgattttatgtttacctacattggttgattgtataagaaattgtatccacaagatactaggatacacagtaattgcaatgcctgaagtagaaggagaagaaatacaaccacaaatggaattgaggagaaatggtaggcaatgtggcatgtctgaaaaagaggaggaatgatgaagtatctcagacttattttataagggagatactgtgctgagttcttccctttgaggaaggtatgtcatatgaatccatttcgaatcaaatcaaactaataaagtatgtattgtaaggtaaaaggaaaagacaaagaagaagaagaaagaagaaagccttcagtacatttatattggctcatgtccaatatgaccgccatgttgacattgattattgactagttattaatagtaatcaattacggggtcattagttcatagcccatatatggagttccgcgttacataacttacggtaattggcccgcctgctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccggccccctattgacgtcaatgacggtaaatggcccgcctggcattatgcccagtacatgaccttacgggactttggtacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagactctataggcacacccctttggctcttatgcatgctatactgtttttggcttggggcctatacacccccgctccttatgctataggtgatggtatagcttagcctataggtgtgggttattgaccattattgaccactcccctattggtgacgatactttccattactaatccataacatggctctttgccacaactatctctattggctatatgccaatactctgtccttcagagactgacacggactctgtatttttacaggatggggtcccatttattatttacaaattcacatatacaacaacgccgtcccccgtgcccgcagtttttattaaacatagcgtgggatctccacgcgaatctcgggtacgtgttccggacatgggctcttctccggtagcggcggagcttccacatccgagccctggtcccatgcctccagcggctcatggtcgctcggcagctccttgctcctaacagtggaggccagacttaggcacagcacaatgcccaccaccaccagtgtgccgcacaaggccgtggcggtagggtatgtgtctgaaaatgagctcggagattgggctcgcaccgtgacgcagatggaagacttaaggcagcggcagaagaagatgcaggcagctgagttgttgtattctgataagagtcagaggtaactcccgttgcggttctgttaacggtggagggcagtgtagtctgagcagtactcgttgctgccgcgcgcgccaccagacataatagctgacagactaacagactgttcctttccatgggtcttttctgcagtcaccgtcgtcgaagcttatgaccatgattacggattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatgcacggttacgatgcgcccatctacaccaacgtaacctatcccattacggtcaatccgccgtttgttcccacggagaatccgacgggttgttactcgctcacatttaatgttgatgaaagctggctacaggaaggccagacgcgaattatttttgatggcgttaactcggcgtttcatctgtggtgcaacgggcgctgggtcggttacggccaggacagtcgtttgccgtctgaatttgacctgagcgcatttttacgcgccggagaaaaccgcctcgcggtgatggtgctgcgttggagtgacggcagttatctggaagatcaggatatgtggcggatgagcggcattttccgtgacgtctcgttgctgcataaaccgactacacaaatcagcgatttccatgttgccactcgctttaatgatgatttcagccgcgctgtactggaggctgaagttcagatgtgcggcgagttgcgtgactacctacgggtaacagtttctttatggcagggtgaaacgcaggtcgccagcggcaccgcgcctttcggcggtgaaattatcgatgagcgtggtggttatgccgatcgcgtcacactacgtctgaacgtcgaaaacccgaaactgtggagcgccgaaatcccgaatctctatcgtgcggtggttgaactgcacaccgccgacggcacgctgattgaagcagaagcctgcgatgtcggtttccgcgaggtgcggattgaaaatggtctgctgctgctgaacggcaagccgttgctgattcgaggcgttaaccgtcacgagcatcatcctctgcatggtcaggtcatggatgagcagacgatggtgcaggatatcctgctgatgaagcagaacaactttaacgccgtgcgctgttcgcattatccgaaccatccgctgtggtacacgctgtgcgaccgctacggcctgtatgtggtggatgaagccaatattgaaacccacggcatggtgccaatgaatcgtctgaccgatgatccgcgctggctaccggcgatgagcgaacgcgtaacgcgaatggtgcagcgcgatcgtaatcacccgagtgtgatcatctggtcgctggggaatgaatcaggccacggcgctaatcacgacgcgctgtatcgctggatcaaatctgtcgatccttcccgcccggtgcagtatgaaggcggcggagccgacaccacggccaccgatattatttgcccgatgtacgcgcgcgtggatgaagaccagcccttcccggctgtgccgaaatggtccatcaaaaaatggctttcgctacctggagagacgcgcccgctgatcctttgcgaatacgcccacgcgatgggtaacagtcttggcggtttcgctaaatactggcaggcgtttcgtcagtatccccgtttacagggcggcttcgtctgggactgggtggatcagtcgctgattaaatatgatgaaaacggcaacccgtggtcggcttacggcggtgattttggcgatacgccgaacgatcgccagttctgtatgaacggtctggtctttgccgaccgcacgccgcatccagcgctgacggaagcaaaacaccagcagcagtttttccagttccgtttatccgggcaaaccatcgaagtgaccagcgaatacctgttccgtcatagcgataacgagctcctgcactggatggtggcgctggatggtaagccgctggcaagcggtgaagtgcctctggatgtcgctccacaaggtaaacagttgattgaactgcctgaactaccgcagccggagagcgccgggcaactctggctcacagtacgcgtagtgcaaccgaacgcgaccgcatggtcagaagccgggcacatcagcgcctggcagcagtggcgtctggcggaaaacctcagtgtgacgctccccgccgcgtcccacgccatcccgcatctgaccaccagcgaaatggatttttgcatcgagctgggtaataagcgttggcaatttaaccgccagtcaggctttctttcacagatgtggattggcgataaaaaacaactgctgacgccgctgcgcgatcagttcacccgtgcaccgctggataacgacattggcgtaagtgaagcgacccgcattgaccctaacgcctgggtcgaacgctggaaggcggcgggccattaccaggccgaagcagcgttgttgcagtgcacggcagatacacttgctgatgcggtgctgattacgaccgctcacgcgtggcagcatcaggggaaaaccttatttatcagccggaaaacctaccggattgatggtagtggtcaaatggcgattaccgttgatgttgaagtggcgagcgatacaccgcatccggcgcggattggcctgaactgccagctggcgcaggtagcagagcgggtaaactggctcggattagggccgcaagaaaactatcccgaccgccttactgccgcctgttttgaccgctgggatctgccattgtcagacatgtataccccgtacgtcttcccgagcgaaaacggtctgcgctgcgggacgcgcgaattgaattatggcccacaccagtggcgcggcgacttccagttcaacatcagccgctacagtcaacagcaactgatggaaaccagccatcgccatctgctgcacgcggaagaaggcacatggctgaatatcgacggtttccatatggggattggtggcgacgactcctggagcccgtcagtatcggcggaattccagctgagcgccggtcgctaccattaccagttggtctggtgtcaaaaataactcgatcgaccagagctgagatcctacaggagtccagggctggagagaaaacctctgaagaggatgatgacagagttagaagatcgcttcaggaagctatttggcacgacttctacaacgggagacagcacagtagattctgaagatgaacctcctaaaaaagaaaaaagggtggactgggatgagtattggaaccctgaagaaatagaaagaatgcttatggactagggactgtttacgaacaaatgataaaaggaaatagctgagcatgactcatagttaaagcgctagcagctgcctaaccgcaaaaccacatcctatggaaagcttgctaatgacgtataagttgttccattgtaagagtatataaccagtgctttgtgaaacttcgaggagtctctttgttgaggacttttgagttctcccttgaggctcccacagatacaataaatatttgagattgaaccctgtcgagtatctgtgtaatcttttttacctgtgaggtctcggaatccgggccgagaacttcgcagcggccgctcgagcatgcatctagagggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgaggggggatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattttaacaaaatattaacgtttacaatttaaatatttgcttatacaatcttcctgtttttggggcttttctgattatcaaccggggtgggtaccgagctcgaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctcccgggagcttggatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctggatcccgtcgacctcgagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtg

SEQ ID NO: 9 - DNA, 1200 bp, Artificial Sequence
  Description of Artificial Sequence/note = synthetic construct
atggacagcagcgctgcccccacgaacgccagcaattgcactgatgccttggcgtactcaagttgctccccagcacccagccccggttcctggatcaacttgtcccacttagatggcaacctgtccgacccatgcggtccgaaccgcaccgacctgggcgggagagacagcctgtgccctccgaccggcagtccctccatgatcacggccatcacgatcatggccctctactccatcgtgtgcgtggtggggctcttcggaaacttcctggtcatgtatgtgattgtcagatacaccaagatgaagactgccaccaacatctacattttcaaccttgctctggcagatgccttagccaccagtaccctgcccttccagagtgtgaattacctaatgggaacatggccatttggaaccatcctttgcaagatagtgatctccatagattactataacatgttcaccagcatattcaccctctgcaccatgagtgttgatcgatacattgcagtctgccaccctgtcaaggccttagatttccgtactccccgaaatgccaaaattatcaatgtctgcaactggatcctctcttcagccattggtcttcctgtaatgttcatggctacaacaaaatacaggcaaggttccatagattgtacactaacattctctcatccaacctggtactgggaaaacctgctgaagatctgtgttttcatcttcgccttcattatgccagtgctcatcattaccgtgtgctatggactgatgatcttgcgcctcaagagtgtccgcatgctctctggctccaaagaaaaggacaggaatcttcgaaggatcaccaggatggtgctggtggtggtggctgtgttcatcgtctgctggactcccattcacatttacgtcatcattaaagccttggttacaatcccagaaactacgttccagactgtttcttggcacttctgcattgctctaggttacacaaacagctgcctcaacccagtcctttatgcatttctggatgaaaacttcaaacgatgcttcagagagttctgtatcccaacctcttccaacattgagcaacaaaactccactcgaattcgtcagaacactagagaccacccctccacggccaatacagtggatagaactaatcatcagctagaaaatctggaagcagaaactgctccgttgccc

SEQ ID NO: 10 - protein, 400 aa, Artificial Sequence
  Description of Artificial Sequence/note = synthetic construct
MDSSAAPTNASNCTDALAYSSCSPAPSPGSWINLSHLDGNLSDPCGPNRTDLGGRDSLCPPTGSPSMITAITIMALYSIVCVVGLFGNFLVMYVIVRYTKMKTATNIYIFNLALADALATSTLPFQSVNYLMGTWPFGTILCKIVISIDYYNMFTSIFTLCTMSVDRYIAVCHPVKALDFRTPRNAKIINVCNWILSSAIGLPVMFMATTKYRQGSIDCTLTFSHPTWYWENLLKICVFIFAFIMPVLIITVCYGLMILRLKSVRMLSGSKEKDRNLRRITRMVLVVVAVFIVCWTPIHIYVIIKALVTIPETTFQTVSWHFCIALGYTNSCLNPVLYAFLDENFKRCFREFCIPTSSNIEQQNSTRIRQNTRDHPSTANTVDRTNHQLENLEAETAPLP

SEQ ID NO: 11 - DNA, 1986 bp, Artificial Sequence
  Description of Artificial Sequence/note = synthetic construct
tcctcctctgctcgcccaatccttccaaccccctatggtggtatggctgacacagaaaatgtctgctcctgtatgggacatttgcccctcttctccaaatataagacaggatgaggcctagcttttgctgctccaaagttttaaaagaacacattgcacggcatttagggactctaaagggtggaggaggaatgagggaattgcatcatgccaaggctggtcctcatccatcactgcttccagggcccagagtggcttccaggaggtattcttacaaaggaagcccgatctgtagctaacactcagagcccattttcctgcgttaacccctcccgacctcatatacaggagtaacatgatcagtgacctgggggagctggccaaactgcgggacctgcccaagctgagggccttggtgctgctggacaacccctgtgccgatgagactgactaccgccaggaggccctggtgcagatggcacacctagagcgcctagacaaagagtactatgaggacgaggaccgggcagaagctgaggagatccgacagaggctgaaggaggaacaggagcaagaactcgacccggaccaagacatggaaccgtacctcccgccaacttagtggctcctctagcctgcagggacagtaaaggtgatggcaggaaggcagcccccggaggtcaaaggctgggcacgcgggaggagaggccagagtcagaggctgcgggtatctcagatatgaaggaaagatgagagaggctcaggaagaggtaagaaaagacacaagagaccagagaagggagaagaattagagagggaggcagaggaccgctgtctctacagacatagctggtagagactgggaggaagggatgaaccctgagcgcatgaagggaaggaggtggctggtggtatatggaggatgtagctgggccagggaaaagatcctgcactaaaaatctgaagctaaaaataacaggacacggggtggagaggcgaaaggagggcagattgaggcagagagactgagaggcctggggatgtgggcattccggtagggcacacagttcacttgtcttctctttttccaggaggccaragatgctgacctcaagaactcataataccccagtggggaccaccgcattcatagccctgttacaagaagtgggagatgttcctttttgtcccagactggaaatccattacatcccgaggctcaggttctgtggtggtcatctctgtgtggcttgttctgtgggcctacctaaagtcctaagcacagctctcaagcagatccgaggcgactaagatgctagtaggggttgtctggagagaagagccgaggaggtgggctgtgatggatcagttcagctttcaaataaaaaggcgtttttatattctgtgtcgagttcgtgaacccctgtggtgggcttctccatctgtctgggttagtacctgccactatactggaataaggggacgcctgcttccctcgagttggctggacaaggttatgagcatccgtgtacttatggggttgccagcttggtcctggatcgcccgggcccttcccccacccgttcggttccccaccaccacccgcgctcgtacgtgcgtctccgcctgcagctcttgactcatcggggcccccgggtcacatgcgctcgctcggctctataggcgccgccccctgcccaccccccgcccgcgctgggagccgcagccgccgccactcctgctctctctgcgccgccgccgtcaccaccgccaccgccaccggctgagtctgcagtcctcgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggccaagct

SEQ ID NO: 12 - DNA, 5982 bp, Artificial Sequence
  Description of Artificial Sequence/note = synthetic construct
atgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctgtgaaacttcgaggagtctctttgttgaggacttttgagttctcccttgaggctcccacagatacaataaatatttgagattgaaccctgtcgagtatctgtgtaatcttttttacctgtgaggtctcggaatccgggccgagaacttcgcagttggcgcccgaacagggacttgattgagagtgattgaggaagtgaagctagagcaatagaaagctgttaagcagaactcctgctgacctaaatagggaagcagtagcagacgctgctaacagtgagtatctctagtgaagcggactcgagctcataatcaagtcattgtttaaaggcccagataaattacatctggtgactcttcgcggaccttcaagccaggagattcgccgagggacagtcaacaaggtaggagagattctacagcaacatggggaatggacaggggcgagattggaaaatggccattaagagatgtagtaatgttgctgtaggagtaggggggaagagtaaaaaatttggagaagggaatttcagatgggccattagaatggctaatgtatctacaggacgagaacctggtgatataccagagactttagatcaactaaggttggttatttgcgatttacaagaaagaagagaaaaatttggatctagcaaagaaattgatatggcaattgtgacattaaaagtctttgcggtagcaggacttttaaatatgacgggtgtctactgctgctgcagctgaaaatatgtattctcaaatgggattagacactaggccatctatgaaagaagcaggtggaaaagaggaaggccctccacaggcatatcctattcaaacagtaaatggagtaccacaatatgtagcacttgacccaaaaatggtgtccatttttatggaaaaggcaagagaaggactaggaggtgaggaagttcaactatggtttactgccttctctgcaaatttaacacctactgacatggccacattaataatggccgcaccagggtgcgctgcagataaagaaatattggatgaaagcttaaagcaactgacagcagaatatgatcgcacacatccccctgatgctcccagaccattaccctattttactgcagcagaaattatgggtataggattaactcaagaacaacaagcagaagcaagatttgcaccagctaggatgcagtgtagagcatggtatctcgaggcattaggaaaattggctgccataaaagctaagtctcctcgagctgtgcagttaagacaaggagctaaggaagattattcatcctttatagacagattgtttgcccaaatagatcaagaacaaaatacagctgaagttaagttatatttaaaacagtcattgagcatagctaatgctaatgcagactgtaaaaaggcaatgagccaccttaagccagaaagtaccctagaagaaaagttgagagcttgtcaagaaataggctcaccaggatataaaatgcaactcttggcagaagctcttacaaaagttcaagtagtgcaatcaaaaggatcaggaccagtgtgttttaattgtaaaaaaccaggacatctagcaagacaatgtagagaagtgaaaaaatgtaataaatgtggaaaacctggtcatgtagctgccaaatgttggcaaggaaatagaaagaattgtacaagggaagaaagggatacaacaattacaaaagtgggaagattgggtaggatggataggaaatattccacaatatttaaagggactattgggaggtatcttgggaataggattaggagtgttattattgattttatgtttacctacattggttgattgtataagaaattgtatccacaagatactaggatacacagtaattgcaatgcctgaagtagaaggagaagaaatacaaccacaaatggaattgaggagaaatggtaggcaatgtggcatgtctgaaaaagaggaggaatgatgaagtatctcagacttattttataagggagatactgtgctgagttcttccctttgaggaaggtatgtcatatgaatccatttcgaaatcaaattagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggtaaaaaagaaaaaagggtggactgggatgagtattggaaccctgaagaaatagaaagaatgcttatggactagggactgtttacgaacaaatgataaaaggaaatagctgagcatgactcatagttaaagcgctagcagctgcctaaccgcaaaaccacatcctatggaaagcttgctaatgacgtataagttgttccattgtaagagtatataaccagtgctttgtgaaacttcgaggagtctctttgttgaggacttttgagttctcccttgaggctcccacagatacaataaatatttgagattgaaccctgtcgagtatctgtgtaatcttttttacctgtgaggtctcggaatccgggccgagaacttcgcagcggccgctcatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctggatcccgtcgacctcgagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtg

SEQ ID NO: 13 - DNA, 13361 bp, Artificial Sequence
  Description of Artificial Sequence/note = synthetic construct
tggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttaactggcttatcgaaattaatacgactcactatagggagacccaagcttggtaccgagctcggatccactagtaacggccgccagtgtgctggaattctgcagatatccatcacactggcggcccataatcaagtcattgtttaaaggcccagataaattacatctggtgactcttcgcggaccttcaagccaggagattcgccgagggacagtcaacaaggtaggagagattctacagcaacatggggaatggacaggggcgagattggaaaatggccattaagagatgtagtaatgttgctgtaggagtaggggggaagagtaaaaaatttggagaagggaatttcagatgggccattagaatggctaatgtatctacaggacgagaacctggtgatataccagagactttagatcaactaaggttggttatttgcgatttacaagaaagaagagaaaaatttggatctagcaaagaaattgatatggcaattgtgacattaaaagtctttgcggtagcaggacttttaaatatgacggtgtctactgctgctgcagctgaaaatatgtattctcaaatgggattagacactaggccatctatgaaagaagcaggtggaaaagaggaaggccctccacaggcatatcctattcaaacagtaaatggagtaccacaatatgtagcacttgacccaaaaatggtgtccatttttatggaaaaggcaagagaaggactaggaggtgaggaagttcaactatggtttactgccttctctgcaaatttaacacctactgacatggccacattaataatggccgcaccagggtgcgctgcagataaagaaatattggatgaaagcttaaagcaactgacagcagaatatgatcgcacacatccccctgatgctcccagaccattaccctattttactgcagcagaaattatgggtataggattaactcaagaacaacaagcagaagcaagatttgcaccagctaggatgcagtgtagagcatggtatctcgaggcattaggaaaattggctgccataaaagctaagtctcctcgagctgtgcagttaagacaaggagctaaggaagattattcatcctttatagacagattgtttgcccaaatagatcaagaacaaaatacagctgaagttaagttatatttaaaacagtcattgagcatagctaatgctaatgcagactgtaaaaaggcaatgagccaccttaagccagaaagtaccctagaagaaaagttgagagcttgtcaagaaataggctcaccaggatataaaatgcaactcttggcagaagctcttacaaaagttcaagtagtgcaatcaaaaggatcaggaccagtgtgttttaattgtaaaaaaccaggacatctagcaagacaatgtagagaagtgaaaaaatgtaataaatgtggaaaacctggtcatgtagctgccaaatgttggcaaggaaatagaaagaattcgggaaactggaaggcggggcgagctgcagccccagtgaatcaaatgcagcaagcagtaatgccatctgcacctccaatggaggagaaactattggatttataaattataataaagtaggtactactacaacattagaaaagaggccagaaatactcatatttgtaaatggatatcctataaaatttttattagacacaggagcagatataacaattttaaataggagagattttcaagtaaaaaattctatagaaaatggaaggcaaaatatgattggagtaggaggaggaaagagaggaacaaattatattaatgtacatttagagattagagatgaaaattataagacacaatgtatatttggtaatgtttgtgtcttagaagataactcattaatacaaccattattagggagagataatatgattaaattcaatattaggttagtaatggctcaaatttctgataagattccagtagtaaaagtaaaaatgaaggatcctaataaaggacctcaaataaaacaatggccattaacaaatgaaaaaattgaagccttaacagaaatagtagaaagactagaaagagaagggaaagtaaaaagagcagatccaaataatccatggaatacaccagtatttgctataaaaaagaaaagtggaaaatggagaatgctcatagattttagagaattaaacaaactaactgagaaaggagcagaggtccagttgggactacctcatcctgctggtttacaaataaaaaaacaagtaacagtattagatataggggatgcatatttcaccattcctcttgatccagattatgctccttatacagcatttactttacctagaaaaaataatgcgggaccaggaaggagatttgtgtggtgtagtctaccacaaggctggattttaagtccattgatatatcaaagtacattagataatataatacaaccttttattagacaaaatcctcaattagatatttaccaatatatggatgacatttatataggatcaaatttaagtaaaaaggagcataaagaaaaggtagaagaattaagaaaattactattatggtggggatttgaaactccagaagataaattacaggaagaacccccatatacatggatgggttatgaattacatccattaacatggacaatacaacagaaacagttagacattccagaacagcccactctaaatgagttgcaaaaattagcaggaaaaattaattgggctagccaagctattccagacttgagtataaaagcattaactaacatgatgagaggaaatcaaaacctaaattcaacaagacaatggactaaagaagctcgactggaagtacaaaaggcaaaaaaggctatagaagaacaagtacaactaggatactatgaccccagtaaggagttatatgctaaattaagtttggtgggaccacatcaaataagttatcaagtatatcagaaggatccagaaaagatactatggtatggaaaaatgagtagacaaaagaaaaaggcagaaaatacatgtgatatagccttaagagcatgctataagataagagaagagtctattataagaataggaaaagaaccaagatatgaaatacctacttctagagaagcctgggaatcaaatttaattaattcaccatatcttaaggccccacctcctgaggtagaatatatccatgctgctttgaatataaagagagcgttaagtatgataaaagatgctccaataccaggagcagaaacatggtatatagatggaggtagaaagctaggaaaagcagcaaaagcagcctattggacagatacaggaaagtggcaagtgatggaattagaaggcagtaatcagaaggcagaaatacaagcattattattggcattaaaagcaggatcagaggagatgaatattataacagattcacaatatgttataaatattattcttcaacaaccagatatgatggagggaatctggcaagaagttttagaagaattggagaagaaaacagcaatatttatagattgggtcccaggacataaaggtattccaggaaatgaggaagtagataagctttgtcaaacaatgatgataatagaaggggatgggatattagataaaaggtcagaagatgcaggatatgatttattagctgcaaaagaaatacatttattgccaggagaggtaaaagtaataccaacaggggtaaagctaatgttgcctaaaggatattggggattaataataggaaaaagctcgatagggagtaaaggattggatgtattaggaggggtaatagacgaaggatatcgaggtgaaattggagtaataatgattaatgtatcaagaaaatcaatcaccttaatggaacgacaaaagatagcacaattaataatattgccttgtaaacatgaagtattagaacaaggaaaagtagtaatggattcagagagaggagacaatggttatgggtcaacaggagtattctcctcttgggttgacagaattgaggaagcagaaataaatcatgaaaaatttcactcagatccacagtacttaaggactgaatttaatttacctaaaatggtagcagaagagataagacgaaaatgcccagtatgcagaatcagaggagaacaagtgggaggacaattgaaaatagggcctggtatctggcaaatggattgcacacactttgatggcaaaataattcttgtgggtatacatgtggaatcaggatatatatgggcacaaataatttctcaagaaactgctgactgtacagttaaagctgtcttacaattgttgagtgctcataatgttactgaattacaaacagataatggaccaaattttaaaaatcaaaagatggaaggagtactcaattacatgggtgtgaaacataagtttggtatcccagggaacccacagtcacaagcattagttgaaaatgtaaatcatacattaaaagtttggattcggaaatttttgcctgaaacaacctccttggataatgccttatctctcgctgtacatagtctcaattttaaaagaagaggtaggataggagggatggccccttatgaattattagcacaacaagaatccttaagaatacaagattatttttctgcaataccacaaaaattgcaagcacagtggatttattataaagatcaaaaagataagaaatggaaaggaccaatgagagtagaatactggggacagggatcagtattattaaaggatgaagagaagggatattttcttatacctaggagacacataaggagagttccagaaccctgcgctcttcctgaaggggatgagtgaagaagattggcaggtaagtagaagactctttgcagtgctccaaggaggagtaaatagcgctatgctatacatatctaggctacctccggatgaaagagaaaagtataaaaaagacttcaagaaaagactttttgacacagaaacaggatttataaagagactacggaaagctgaaggaataaaatggagctttcatactagagattattacataggatatgtcagagaaatggtggcaggatccactacatcattaagtctaaggatgtatatatatataagtaacccactatggcattctcagtatcgtccaggtttgaaaaatttcaataaggaatggccttttgtaaatatgtggataaaaacaggatttatgtgggatgatattgaaaaacaaaatatttgtataggaggagaagtttcaccaggatggggaccagggatggtaggtatagcaataaaagcttttagttgtggcgaaagaaagattgaggctactcctgtaatgattataagaggagaaatagatccaaaaaaatggtgcggagattgttggaatttaatgtgtcttagaaactcacctccaaagactttacaaagactcgctatgttggcgtgtggcgtgccggctaagaagtggcgaggatgctgtaatcaacgctttgtttctccttacagaacgcctgctgatttagaggtcattcaatccaagcccagctggaacctgttatggtcgggagaattatgaatggaagacataatagtattattcaatagggtcactgagaaactagaaaaagaattagctatcagaatatttgtattagcacatcaattagaaagggacaaagctattagattactacaaggattattttggagatatagatttaagaaaccccgagtagattattgtttatgttggtggtgttgcaaattctattattggcagttgcaatctacattatcaataactactgcttagaaatatttagattaatatttcatttgcaacaataagaatggcagaaggatttgcagccaatagacaatggataggactagaagaagctgaagagttattagattttgatatagcaacacaaatgagtgaagaaggaccactaaatccaggagtaaacccatttagggtacctggaataacagaaaaagaaaagcaaaactactgtaacatattacaacctaagttacaagatctaaggaacgaaattcaagaggtaaaactggaagaaggaaatgcaggtaagtttagaagagcaagatttttaaggtattctgatgaaagtgtattgtccctggttcatgcgttcataggatattgtatatatttaggtaatcgaaataagttaggatctttaagacatgacattgatatagaagcaccccaagaagagtgttataataatagagagaagggtacaactgacaatataaaatatggtagacgatgttgcctaggaacggtgactttgtacctgattttatttataggaataataatatattcacagacaaccaacgctcaggtagtatggagacttccaccattagtagtcccagtagaagaatcagaaataattttttgggattgttgggcaccagaagaacccgcctgtcaggactttcttggggcaatgatacatctaaaagctaagacaaatataagtatacgagagggacctaccttggggaattgggctagagaaatatgggcaacattattcaaaaaggctactagacaatgtagaagaggcagaatatggaaaagatggaatgagactataacaggaccatcaggatgtgctaataacacatgttataatgtttcagtaatagtacctgattatcagtgttatttagatagagtagatacttggttacaagggaaaataaatatatcattatgtctaacaggaggaaaaatgttgtacaataaagttacaaaacaattaagctattgtacagacccattacaaatcccactgatcaattatacatttggacctaatcaaacatgtatgtggaatacttcacaaattcaggaccctgaaataccaaggccgcggccctggcaacccatcaagaagctgtagaaaaggtgactgaagccttaaagataaacaacttaagattagttacattagagcatcaagtactagtaataggattaaaagtagaagctatggaaaaatttttgtatacagctttcgctatgcaagaattaggatgtaatcaaaatcaatttttctgcaaaatccctcctgagttgtggacaaggtataatatgactataaatcaaacaatatggaatcatggaaatataactttgggggaatggtataaccaaacaaaagatttacaacaaaagttttatgaaataataatggacatagaacaaaataatgtacaagggaagaaagggatacaacaattacaaaagtgggaagattgggtaggatggataggaaatattccacaatatttaaagggactattgggaggtatcttgggaataggattaggagtgttattattgattttatgtttacctacattggttgattgtataagaaattgtatccacaagatactaggatacacagtaattgcaatgcctgaagtagaaggagaagaaatacaaccacaaatggaattgaggagaaatggtaggcaatgtggcatgtctgaaaaagaggaggaatgatgaagtatctcagacttattttataagggagatactgtgctgagttcttccctttgaggaaggtatgtcatatgaatccatttcgaatcaaatcaaactaataaagtatgtattgtaaggtaaaaggaaaagacaaagaagaagaagaaagaagaaagccttcaagaggatgatgacagagttagaagatcgcttcaggaagctatttggcacgacttctacaacgggagacagcacagtagattctgaagatgaacctcctaaaaaagaaaaaagggtggactgggatgagtattggaaccctgaagaaatagaaagaatgcttatggactagggactgtttacgaacaaatgataaaaggaaatagctgactagagggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgaggggggatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattttaacaaaatattaacgtttacaatttaaatatttgcttatacaatcttcctgtttttggggcttttctgattatcaaccggggtgggtaccgagctcgaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctcccgggagcttggatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctggatcccgtcgacctcgagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccactc

SEQ ID NO: 14 - DNA, 9569 bp, Artificial Sequence
  Description of Artificial Sequence/note = synthetic construct
atgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctgtgaaacttcgaggagtctctttgttgaggacttttgagttctcccttgaggctcccacagatacaataaatatttgagattgaaccctgtcgagtatctgtgtaatcttttttacctgtgaggtctcggaatccgggccgagaacttcgcagttggcgcccgaacagggacttgattgagagtgattgaggaagtgaagctagagcaatagaaagctgttaagcagaactcctgctgacctaaatagggaagcagtagcagacgctgctaacagtgagtatctctagtgaagcggactcgagctcataatcaagtcattgtttaaaggcccagataaattacatctggtgactcttcgcggaccttcaagccaggagattcgccgagggacagtcaacaaggtaggagagattctacagcaacatggggaatggacaggggcgagattggaaaatggccattaagagatgtagtaatgttgctgtaggagtaggggggaagagtaaaaaatttggagaagggaatttcagatgggccattagaatggctaatgtatctacaggacgagaacctggtgatataccagagactttagatcaactaaggttggttatttgcgatttacaagaaagaagagaaaaatttggatctagcaaagaaattgatatggcaattgtgacattaaaagtctttgcggtagcaggacttttaaatatgacgggtgtctactgctgctgcagctgaaaatatgtattctcaaatgggattagacactaggccatctatgaaagaagcaggtggaaaagaggaaggccctccacaggcatatcctattcaaacagtaaatggagtaccacaatatgtagcacttgacccaaaaatggtgtccatttttatggaaaaggcaagagaaggactaggaggtgaggaagttcaactatggtttactgccttctctgcaaatttaacacctactgacatggccacattaataatggccgcaccagggtgcgctgcagataaagaaatattggatgaaagcttaaagcaactgacagcagaatatgatcgcacacatccccctgatgctcccagaccattaccctattttactgcagcagaaattatgggtataggattaactcaagaacaacaagcagaagcaagatttgcaccagctaggatgcagtgtagagcatggtatctcgaggcattaggaaaattggctgccataaaagctaagtctcctcgagctgtgcagttaagacaaggagctaaggaagattattcatcctttatagacagattgtttgcccaaatagatcaagaacaaaatacagctgaagttaagttatatttaaaacagtcattgagcatagctaatgctaatgcagactgtaaaaaggcaatgagccaccttaagccagaaagtaccctagaagaaaagttgagagcttgtcaagaaataggctcaccaggatataaaatgcaactcttggcagaagctcttacaaaagttcaagtagtgcaatcaaaaggatcaggaccagtgtgttttaattgtaaaaaaccaggacatctagcaagacaatgtagagaagtgaaaaaatgtaataaatgtggaaaacctggtcatgtagctgccaaatgttggcaaggaaatagaaagaattgtacaagggaagaaagggatacaacaattacaaaagtgggaagattgggtaggatggataggaaatattccacaatatttaaagggactattgggaggtatcttgggaataggattaggagtgttattattgattttatgtttacctacattggttgattgtataagaaattgtatccacaagatactaggatacacagtaattgcaatgcctgaagtagaaggagaagaaatacaaccacaaatggaattgaggagaaatggtaggcaatgtggcatgtctgaaaaagaggaggaatgatgaagtatctcagacttattttataagggagatactgtgctgagttcttccctttgaggaaggtatgtcatatgaatccatttcgaatcaaattccccagcatgcctgctattgtcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagtcgaggctgatcagcgagctctgggcaacggagcagtttctgcttccagattttctagctgatgattagttctatccactgtattggccgtggaggggtggtctctagatgttctgacgaattcgagtggagttttgttgctcaatgttggaagaggttgggatacagaactctctgaagcatcgtttgaagttttcatccagaaatgcataaaggactgggttgaggcagctgtttgtgtaacctagagcaatgcagaagtgccaagaaacagtctggaacgtagtttctgggattgtaaccaaggctttaatgatgacgtaaatgtgaatgggagtccagcagacgatgaacacagccaccaccaccagcaccatcctggtgatccttcgaagattcctgtccttttctttggagccagagagcatgcggacactcttgaggcgcaagatcatcagtccatagcacacggtaatgatgagcactggcataatgaaggcgaagatgaaaacacagatcttcagcaggttttcccagtaccaggttggatgagagaatgttagtgtacaatctatggaaccttgcctgtattttgttgtagccatgaacattacaggaagaccaatggctgaagagaggatccagttgcagacattgataattttggcatttcggggagtacggaaatctaaggccttgacagggtggcagactgcaatgtatcgatcaacactcatggtgcagagggtgaatatgctggtgaacatgttatagtaatctatggagatcactatcttgcaaaggatggttccaaatggccatgttcccattaggtaattcacactctggaagggcagggtactggtggctaaggcatctgccagagcaaggttgaaaatgtagatgttggtggcagtcttcatcttggtgtatctgacaatcacatacatgaccaggaagtttccgaagagccccaccacgcacacgatggagtagagggccatgatcgtgatggccgtgatcatggagggactgccggtcggagggcacaggctgtctctcccgcccaggtcggtgcggttcggaccgcatgggtcggacaggttgccatctaagtgggacaagttgacccaggaaccggggctgggtgctggggagcaacttgagtacgccaaggcatcagtgcaattgctggcgttcgtgggggcagcgctgctgtccatggtactgacggccgggcgggcaccgctgtgcgaggtagccaggagcaccgagacttttcgggttccaagcgcctcagccgcttcctttcgccgctgccacagctcctgctgtttcttacagaaaccagtcctggctgagacagagaggagcgtcaggcggaggggaccgagctgagcatctgacattctcctctgcgtatagccccctcccaccttagtagttcacagaggctcatccggaattcgatatcaagcttagcttggccgcgggtacaattccgcagcttttagagcagaagtaacacttccgtacaggcctagaagtaaaggcaacatccactgaggagcagttctttgatttgcaccaccaccggatccgggacctgaaataaaagacaaaaagactaaacttaccagttaactttctggtttttcagttcctcgaggactgcagactcagccggtggcggtggcggtggtgacggcggcggcgcagagagagcaggagtggcggcggctgcggctcccagcgcgggcggggggtgggcagggggcggcgcctatagagccgagcgagcgcatgtgacccgggggccccgatgagtcaagagctgcaggcggagacgcacgtacgagcgcgggtggtggtggggaaccgaacgggtgggggaagggcccgggcgatccaggaccaagctggcaaccccataagtacacggatgctcataaccttgtccagccaactcgagggaagcaggcgtccccttattccagtatagtggcaggtactaacccagacagatggagaagcccaccacaggggttcacgaactcgacacagaatataaaaacgcctttttatttgaaagctgaactgatccatcacagcccacctcctcggctcttctctccagacaacccctactagcatcttagtcgcctcggatctgcttgagagctgtgcttaggactttaggtaggcccacagaacaagccacacagagatgaccaccacagaacctgagcctcgggatgtaatggatttccagtctgggacaaaaaggaacatctcccacttcttgtaacagggctatgaatgcggtggtccccactggggtattatgagttcttgaggtcagcatctytggcctcctggaaaaagagaagacaagtgaactgtgtgccctaccggaatgcccacatccccaggcctctcagtctctctgcctcaatctgccctcctttcgcctctccaccccgtgtcctgttatttttagcttcagatttttagtgcaggatcttttccctggcccagctacatcctccatataccaccagccacctccttcccttcatgcgctcagggttcatcccttcctcccagtctctaccagctatgtctgtagagacagcggtcctctgcctccctctctaattcttctcccttctctggtctcttgtgtcttttcttacctcttcctgagcctctctcatctttccttcatatctgagatacccgcagcctctgactctggcctctcctcccgcgtgcccagcctttgacctccgggggctgccttcctgccatcacctttactgtccctgcaggctagaggagccactaagttggcgggaggtacggttccatgtcttggtccgggtcgagttcttgctcctgttcctccttcagcctctgtcggatctcctcagcttctgcccggtcctcgtcctcatagtactctttgtctaggcgctctaggtgtgccatctgcaccagggcctcctggcggtagtcagtctcatcggcacaggggttgtccagcagcaccaaggccctcagcttgggcaggtcccgcagtttggccagctcccccaggtcactgatcatgttactcctgtatatgaggtcgggaggggttaacgcaggaaaatgggctctgagtgttagctacagatcgggcttcctttgtaagaatacctcctggaagccactctgggccctggaagcagtgatggatgaggaccagccttggcatgatgcaattccctcattcctcctccaccctttagagtccctaaatgccgtgcaatgtgttcttttaaaactttggagcagcaaaagctaggcctcatcctgtcttatatttggagaagaggggcaaatgtcccatacaggagcagacattttctgtgtcagccataccaccatagggggttggaaggattgggcgagcagaggaggaaatggaattgaggagaaatggtaggcaatgtggcatgtctgaaaaagaggaggaatgatgaagtatctcagacttattttataagggagatactgtgctgagttcttccctttgaggaaggtatgtcatatgaatccatttcgaatcaaattcctaaaaaagaaaaaagggtggactgggatgagtattggaaccctgaagaaatagaaagaatgcttatggactagggactgtttacgaacaaatgataaaaggaaatagctgagcatgactcatagttaaagcgctagcagctgcctaaccgcaaaaccacatcctatggaaagcttgctaatgacgtataagttgttccattgtaagagtatataaccagtgctttgtgaaacttcgaggagtctctttgttgaggacttttgagttctcccttgaggctcccacagatacaataaatatttgagattgaaccctgtcgagtatctgtgtaatcttttttacctgtgaggtctcggaatccgggccgagaacttcgcagtgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctggatcccgtcgacctcgagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtg

SEQ ID NO: 15 - protein, 401 aa, Artificial Sequence
  Description of Artificial Sequence/note = synthetic construct
MDSGAVPTNASNCTDPFTHPSSCSPAPSPSSWVNFSHLEGNLSDPCGPNRTELGGSDRLCPSAGSPSMITAIIIMALYSIVCVVGLFGNFLVMYVIVRYTKMKTATNIYIFNLALADALATSTLPFQSVNYLMGTWPFGTILCKIVISIDYYNMFTSIFTLCTMSVDRYIAVCHPVKALDLRTPRNAKIINICNWILSSAIGLPVMFMATTKYRQGSIDCTLTFSHPTWYWENLLKICVFIFAFIMPILIITVCYGLMILRLKSVRMLSGSKEKDRNLRRITRMVLVVVAVFIVCWTPIHIYVIIKALITIPETTFQTVSWHFCIALGYTNSCLNPVLYAFLDENFKRCFREFCIPTSSTIEQQNSTRIRQNTRDHPSTANTVDRTNHQLENLEAETTPLP

SEQ ID NO: 16 - DNA, 1415 bp, Artificial Sequence
  Description of Artificial Sequence/note = synthetic construct
gcctgacgctcctctctggctccgccggggttggtcgctgtaagaaataacaggagctgtggcagcggcgaagacgaagcggctcgcgcgtggaacccgaaaagtcagggtgctcgcggttactcccaacgtggtcccagccggcggtcagcaccatggacagcggcgccgtccccacgaacgccagcaactgcactgatcccttcacacacccttcaagttgctccccagcacctagtcccagctcctgggtcaacttctcccacttagaaggcaacctgtccgacccatgcggtccgaaccgcaccgagctgggagggagcgacagactgtgcccttcggccggcagcccttccatgatcacggccatcatcatcatggccctctactccatcgtgtgcgtggtggggctcttcggaaacttcctggtcatgtatgtgattgtcaggtacaccaaaatgaagactgccaccaacatctatattttcaacctcgccctggcagatgccctggcaaccagtaccctgcctttccagagtgtcaattacctgatgggaacatggccgtttggaaccatcctgtgcaagattgtgatctccatagattactacaatatgttcaccagcatattcaccctctgcaccatgagtgtggatcgctacattgcagtctgccatcctgtcaaggccctggatttacgcactccccgtaatgccaagatcatcaacatctgcaactggatcctctcttcagccattggtctgcctgtgatgttcatggcaacgacaaagtaccggcaaggttccatagattgtacactaacattctctcacccaacgtggtactgggaaaacctgctgaaaatctgtgttttcatctttgccttcatcatgcctatcctcatcattacagtgtgttatgggctgatgatcttacgcctcaagagtgtccgcatgctctctggctccaaagaaaaggacaggaacctgcgaagaatcaccaggatggtgctggtggttgtggctgtgttcattgtctgctggacgcccattcacatctacgtcatcattaaagccttgatcacaatcccggaaactactttccagaccgtttcctggcacttctgcattgctctaggttataccaacagttgcctcaaccccgtcctttatgcatttctggatgaaaacttcaaacgatgcttcagagagttctgtatcccaacttcctccaccattgagcagcaaaactccactcgaattcgtcagaacaccagagaccacccctccacagccaatacggtggataggactaaccatcagctagaaaatctggaagcagaaaccactccgttaccctaactgggtctcataccattcagaccctcactgagcttagacgccacatctatatga

SEQ ID NO: 17 - protein, 398 aa, Artificial Sequence
  Description of Artificial Sequence/note = synthetic construct
MDSSAGPGNISDCSDPLAPASCSPAPGSWLNLSHVDGNQSDPCGPNRTGLGGSHSLCPQTGSPSMVTAITIMALYSIVCVVGLFGNFLVMYVIVRYTKMKTATNIYIFNLALADALATSTLPFQSVNYLMGTWPFGNILCKIVISIDYYNMFTSIFTLCTMSVDRYIAVCHPVKALDFRTPRNAKIVNVCNWILSSAIGLPVMFMATTKYRQGSIDCTLTFSHPTWYWENLLKICVFIFAFIMPVLIITVCYGLMILRLKSVRMLSGSKEKDRNLRRITRMVLVVVAVFIVCWTPIHIYVIIKALITIPETTFQTVSWHFCIALGYTNSCLNPVLYAFLDENFKRCFREFCIPTSSTIEQQNSARIRQNTREHPSTANTVDRTNHQLENLEAETAPLP

SEQ ID NO: 18 - DNA, 2229 bp, Artificial Sequence
  Description of Artificial Sequence/note = synthetic construct
cggatccttagcatccccaaagcgcctccgtgtacttctaaggtgggagggggatacaagcagaggagaatatcggacgctcagacgttccattctgcctgccgctcttctctggttccactagggcttgtccttgtaagaaactgacggagcctagggcagctgtgagaggaagaggctggggcgcctggaacccgaacactcttgagtgctctcagttacagcctaccgagtccgcagcaagcattcagaaccatggacagcagcgccggcccagggaacatcagcgactgctctgaccccttagctcctgcaagttggtccccagcacctggctcctggctcaacttgtcccacgttgatggcaaccagtccgacccatgcggtcctaaccgcacggggcttggcgggagccacagcctgtgccctcagaccggcagcccttccatggtcacagccatcaccatcatggccctctattctatcgtgtgtgtagtgggcctctttggaaacttcctggtcatgtatgtgattgtaagatataccaaaatgaagactgccaccaacatctacattttcaaccttgctctggcagatgccttagccactagcacgctgccctttcagagtgttaactacctgatgggaacgtggccctttggaaacatcctctgcaagatcgtgatctcaatagactactacaacatgttcaccagtatcttcaccctctgcaccatgagtgtagaccgctacattgccgtctgccacccggtcaaggccctggatttccgtaccccccgaaatgccaaaattgtcaatgtctgcaactggatcctctcttctgccattggtctgcccgtaatgttcatggcaaccacaaaatacaggcaggggtccatagattgcaccctcactttctctcatcccacatggtactgggagaacctgctcaaaatctgtgtcttcatcttcgccttcatcatgccggtcctcatcatcactgtgtgttatggactgatgatcttacgactcaagagtgtccgcatgctgtcgggctccaaagaaaaggacaggaacctgcgcaggatcacccggatggtgctggtggtcgtggctgtatttattgtctgctggacccccatccacatctatgtcatcatcaaagcactgatcacgattccagaaaccactttccagactgtttcctggcacttctgcattgccttgggttacacaaacagctgcctgaacccagttctttatgcgttcctggatgaaaacttcaaacgatgttttagagagttctgcatcccaacttcctccacaatcgaacagcaaaactctgctcgaatccgtcaaaacactagggaacacccctccacggctaatacagtggatcgaactaaccaccagctagaaaatctggaagcagaaactgctccattgccctaactgggtcccacgccatccagaccctcgctaaacttagaggctgccatctacttggaatcaggttgctgtcagggtttgtgggaggctctggtttcctggaaaagcatctgatcctgcatcattcaaagtcattcctctctggctattcacgctacacgtcagagacactcagactgtgtcaagcactcagaaggaagagactgcaggccactactgaatccagctcatgtacagaaacatccaatggaccacaatactctgtggtatgtgatttgtgatcaacatagaaggtgacccttccctatgtggaatttttaatttcaaggaaatacttatgatctcatcaagggaaaaatagatgtcacttgttaaattcactgtagtgatgcataaaggaaaagctacctctgacctctagcccagtcaccctctatggaaagttccatagggaatatgtgagggaaaatgttgcttccaaattaaattttcacctttatgttatagtctagttaagacatcaggggcatctctgtttcttggttttgtattgtttgaaagaagacatcttcctccctagctgcgtgttgaaaatgaaagggatttaaaacacagtgtcaactgcagaatagttgattctcgcactgaagggggggggctaatcttcccaattctttccatgtcctccaagtgttcacaaggtcaaactcagagtcacccagtaagctcatcatgccaccattctgagcaaaatccttggattcctgctcagaatggtgg

SEQ ID NO: 19 - protein, 398 aa, Artificial Sequence
  Description of Artificial Sequence/note = synthetic construct
MDSSTGPGNTSDCSDPLAQASCSPAPGSWLNLSHVDGNQSDPCGLNRTGLGGNDSLCPQTGSPSMVTAITIMALYSIVCVVGLFGNFLVMYVIVRYTKMKTATNIYIFNLALADALATSTLPFQSVNYLMGTWPFGTILCKIVISIDYYNMFTSIFTLCTMSVDRYIAVCHPVKALDFRTPRNAKIVNVCNWILSSAIGLPVMFMATTKYRQGSIDCTLTFSHPTWYWENLLKICVGIFAFIMPVLIITVCYGLMILRLKSVRMLSGSKEKDRNLRRITRMVLVVVAVFIVCWTPIHIYVIIKALITIPETTFQTVSWHFCIALGYTNSCLNPVLYAFLDENFKRCFREFCIPTSSTIEQQNSTRVRQNTREHPSTANTVDRTNHQLENLEAETAPLP

SEQ ID NO: 20 - DNA, 1401 bp, Artificial Sequence
  Description of Artificial Sequence/note = synthetic construct
cacgagcttctgcctgccgctcttctctggttccactagggctggtccatgtaagaatctgacggagcctagggcagctgtgagaggaagaggctggggcgcgtggaacccgaaaagtctgagtgctctcagttacagcctacctagtccgcagcaggccttcagcaccatggacagcagcaccggcccagggaacaccagcgactgctcagaccccttagctcaggcaagttgctccccagcacctggctcctggctcaacttgtcccacgttgatggcaaccagtccgatccatgcggtctgaaccgcaccgggcttggcgggaacgacagcctgtgccctcagaccggcagcccttccatggtcacagccattaccatcatggccctctactctatcgtgtgtgtagtgggcctcttcggaaacttcctggtcatgtatgtgattgtaagatacaccaaaatgaagactgccaccaacatctacattttcaaccttgctctggcagacgccttagcgaccagtacactgccctttcagagtgtcaactacctgatgggaacatggcccttcggaaccatcctctgcaagatcgtgatctcaatagattactacaacatgttcaccagcatattcaccctctgcaccatgagcgtggaccgctacattgctgtctgccacccagtcaaagccctggatttccgtaccccccgaaatgccaaaatcgtcaacgtctgcaactggatcctctcttctgccatcggtctgcctgtaatgttcatggcaaccacaaaatacaggcaggggtccatagattgcaccctcacgttctcccacccaacctggtactgggagaacctgctcaaaatctgtgtctttatcttcgctttcatcatgccggtcctcatcatcactgtgtgttacggcctgatgatcttacgactcaagagcgttcgcatgctatcgggctccaaagaaaaggacaggaatctgcgcaggatcacccggatggtgctggtggtcgtggctgtatttatcgtctgctggacccccatccacatctacgtcatcatcaaagcgctgatcacgattccagaaaccacatttcagacggtttcctggcacttctgcattgctttgggttacacgaacagctgcctgaatccagttctttacgccttcctggatgaaaacttcaagcgatgcttcagagagttctgcatcccaacctcgtccacgatcgaacagcaaaactccactcgagtccgtcagaacactagggaacatccctccacggctaatacagtggatcgaactaaccaccagctagaaaatctggaggcagaaactgctccattgccctaactgggtctcacaccatccagaccctcgctaagctt

SEQ ID NO: 21 - protein, 401 aa, Artificial Sequence
  Description of Artificial Sequence/note = synthetic construct
MDSSADPRNASNCTDPFSPSSMCSPVPSPSSWVNFSHLEGNLSDPCIRNRTELGGSDSLCPPTGSPSMVTAITIMALYSIVCVVGLFGNFLVMYVIVRYTKMKTATNIYIFNLALADALATSTLPFQSVNYLMGTWPFGTILCKIVISIDYYNMFTSIFTLCTMSVDRYIAVCHPVKALDFRTPRNAKIINVCNWILSSAIGLPVMFMATTKYRNGSIDCALTFSHPTWYWENLLKICVFIFAFIMPVLIITVCYGLMILRLKSVRMLSGSKEKDRNLRRITRMVLVVVAVFIVCWTPIHIYVIIKALITIPETTFQTVSWHFCIALGYTNSCLNPVLYAFLDENFKRCFREFCIPTSSTIEQQNSARIRQNTRDHPSTANTVDRTNHQLENLEAETAPLP

SEQ ID NO: 22 - DNA, 1881 bp, Artificial Sequence
  Description of Artificial Sequence/note = synthetic construct
atgagctgtggtgtacttctaagatgggagggggcaacaagcagaagataatgtcagaagcttagctctccttctgcctgacgctcctctctggctccgcctgggttggcttctgtaagaagtagcaggagccgtggcgggggctggaggaagcggctgaggcgcgtggaacccgaaaagcccgggtgatcgcggttacctcactgcgtggtcccagccgcccagccgtcagcaccatggacagcagcgctgacccccgaaacgccagcaattgcactgatcccttctcgccctcttcaatgtgctccccagtacctagccccagctcctgggtcaacttctcccacttagaaggcaacctgtccgacccatgcattcggaaccgcaccgagctgggcgggagcgacagcctgtgccctccgaccggcagtccttccatggtcacggccatcaccatcatggccctctactccatcgtgtgcgtggtgggtctcttcggaaacttcctggtcatgtatgtgattgtcagatacaccaaaatgaagactgccaccaacatctatattttcaaccttgctctggcggatgccttagccaccagtaccctacccttccagagtgtcaattacctaatgggaacgtggccgtttggaaccatcctctgcaagatcgtgatctccatagattactacaatatgttcaccagcatattcaccctctgtaccatgagcgtggatcgctacatcgccgtctgccatcccgtcaaggccctggacttccgcactccccgcaacgccaaaatcatcaacgtctgcaactggatcctctcttcagccattggtctgcctgtgatgttcatggcaacaacaaagtaccggaatggttccatagattgtgcactaacattctctcacccaacctggtactgggaaaacctgctgaaaatctgtgttttcatctttgccttcatcatgcctgtcctcatcattacggtgtgttatgggctgatgatcttacgcctcaagagtgttcgcatgctctctggctccaaagaaaaggataggaacctgcgaagaatcaccaggatggtgctggtggttgtggctgtgttcattgtctgctggactcccattcacatttacgtcatcattaaagccttgattacaattccagaaactactttccagactgtgtcctggcacttctgcattgctctaggttatacaaacagctgcctgaacccagtcctttatgcatttctggatgaaaacttcaaacgatgcttcagagagttctgtatcccaacctcctccaccattgagcagcaaaactccgctcgaatccgtcaaaacaccagagaccacccctccacggccaacacggtggacaggaccaaccatcagctagaaaatctggaagcagaaactgctccattgccctaaccaggtgtcatgccattcagatcctcaatgagctaagacagccaccatctacgtggaagcaggttgccatgagaatgtgtgggaggcactattttcctaggaaagtgcctgctctgagtcatcaaatctgtttcctctctggccgctctgctctgcacatgagagggacatccaaactaaatcaagcactaggaaggaaagaactaatccacatggagtttgcctgtgcacataatctcaaggaagatgacccatgggaccgaaacatgctgtggtatgtgcgttgaggtcatcctcaaagatggcccttctgtatgtaatgtgctgttttcaagcaaatgtttacgtcctcatcaaagaaaaaatgtcagttgttaaattcaccatagtaacttgtaaaggctacctctgatcgaagcatcttatgtggaaatccaag

SEQ ID NO: 23 - protein, 372 aa, Artificial Sequence
  Description of Artificial Sequence/note = synthetic construct
MEPAPSAGAELQPPLFANASDAYPSAFPSAGANASGPPGARSASSLALAIAITALYSAVCAVGLLGNVLVMFGIVRYTKMKTATNIYIFNLALADALATSTLPFQSAKYLMETWPFGELLCKAVLSIDYYNMFTSIFTLTMMSVDRYIAVCHPVKALDFRTPAKAKLINICIWVLASGVGVPIMVMAVTRPRDGAVVCMLQFPSPSWYWDTVTKICVFLFAFVVPILIITVCYGLMLLRLRSVRLLSGSKEKDRSLRRITRMVLVVVGAFVVCWAPIHIFVIVWTLVDIDRRDPLVVAALHLCIALGYANSSLNPVLYAFLDENFKRCFRQLCRKPCGRPDPSSFSRAREATARERVTACTPSDGPGGGAAA

SEQ ID NO: 24 - DNA, 1773 bp, Artificial Sequence; identical to SEQ ID NO: 6
  Description of Artificial Sequence/note = synthetic construct

SEQ ID NO: 25 - protein, 228 aa, Artificial Sequence
  Description of Artificial Sequence/note = synthetic construct
GIVRYTKMKTATNIYIFNLALADALATSTLPFQSAKYLMETWPFGELLCKAVLSIDYYNMFTSIFTLTMMSVDRYIAVCHPVKALDFRTPAKAKLINICIWVLASGVGVPIMVMAVTRPRDGAVVCMLQFPSPSWYWDTVTKICVFLFAFVVPILVITVCYGLMLLRLRSVRLLSGSKEKDRSLRRITRMVLVVVGAFVVCWAPIHIFVIVWTLVDIDRRDPLVVAAL

SEQ ID NO: 26 - protein, 372 aa, Artificial Sequence
  Description of Artificial Sequence/note = synthetic construct
MEPVPSARAELQFSLLANVSDTFPSAFPSASANASGSPGARSASSLALAIAITALYSAVCAVGLLGNVLVMFGIVRYTKLKTATNIYIFNLALADALATSTLPFQSAKYLMETWPFGELLCKAVLSIDYYNMFTSIFTLTMMSVDRYIAVCHPVKALDFRTPAKAKLINICIWVLASGVGVPIMVMAVTQPRDGAVVCTLQFPSPSWYWDTVTKICVFLFAFVVPILIITVCYGLMLLRLRSVRLLSGSKEKDRSLRRITRMVLVVVGAFVVCWAPIHIFVIVWTLVDINRRDPLVVAALHLCIALGYANSSLNPVLYAFLDENFKRCFRQLCRAPCGGQEPGSLRRPRQATARERVTACTPSDGPGGGAAA

SEQ ID NO: 27 - protein, 372 aa, Artificial Sequence
  Description of Artificial Sequence/note = synthetic construct
MELVPSARAELQSSPLVNLSDAFPSAFPSAGANASGSPGARSASSLALAIAITALYSAVCAVGLLGNVLVMFGIVRYTKLKTATNIYIFNLALADALATSTLPFQSAKYLMETWPFGELLCKAVLSIDYYNMFTSIFTLTMMSVDRYIAVCHPVKALDFRTPAKAKLINICIWVLASGVGVPIMVMAVTQPRDGAVVCMLQFPSPSWYWDTVTKICVFLFAFVVPILIITVCYGLMLLRLRSVRLLSGSKEKDRSLRRITRMVLVVVGAFVVCWAPIHIFVIVWTLVDINRRDPLVVAALHLCIALGYANSSLNPVLYAFLDENFKRCFRQLCRTPCGRQEPGSLRRPRQATTRERVTACTPSDGPGGGAAA

SEQ ID NO: 28 - DNA, 2219 bp, Artificial Sequence
  Description of Artificial Sequence/note = synthetic construct
ctctaaaggctgggtccctgcgcccagggcgcacggtggagacggacacggcggcgccatggagctggtgccctctgcccgtgcggagctgcagtcctcgcccctcgtcaacctctcggacgcctttcccagcgccttccccagcgcgggcgccaatgcgtcggggtcgccgggagcccgtagtgcctcgtccctcgccctagccatcgccatcaccgcgctctactcggctgtgtgcgcagtggggcttctgggcaacgtgctcgtcatgtttggcatcgtccggtacaccaaattgaagaccgccaccaacatctacatcttcaatctggctttggctgatgcgctggccaccagcacgctgcccttccagagcgccaagtacttgatggaaacgtggccgtttggcgagctgctgtgcaaggctgtgctctccattgactactacaacatgttcactagcatcttcaccctcaccatgatgagcgtggaccgctacattgctgtctgccatcctgtcaaagccctggacttccggacaccagccaaggccaagctgatcaatatatgcatctgggtcttggcttcaggtgtcggggtccccatcatggtcatggcagtgacccaaccccgggatggtgcagtggtatgcatgctccagttccccagtcccagctggtactgggacactgtgaccaagatctgcgtgttcctctttgccttcgtggtgccgatcctcatcatcacggtgtgctatggcctcatgctactgcgcctgcgcagcgtgcgtctgctgtccggttccaaggagaaggaccgcagcctgcggcgcatcacgcgcatggtgctggtggtggtgggcgccttcgtggtgtgctgggcgcccatccacatcttcgtcatcgtctggacgctggtggacatcaatcggcgcgacccacttgtggtggccgcactgcacctgtgcattgcgctgggctacgccaacagcagcctcaacccggttctctacgccttcctggacgagaacttcaagcgctgcttccgccagctctgtcgcacgccctgcggccgccaagaacccggcagtctccgtcgtccccgccaggccaccacgcgtgagcgtgtcactgcctgcaccccctccgacggcccgggcggtggcgctgccgcctgacctacccgaccttccccttaaacgcccctcccaagtgaagtgatccagaggccacaccgagctccctgggaggctgtggccaccaccaggacagctagaattgggcctgcacagaggggaggcctcctgtggggacggggcctgagggatcaaaggctccaggttggaacggtgggggtgaggaagcagagctggtgattcctaaactgtatccattagtaaggcctctccaatgggacagagcctccgccttgagataacatcgggttctggcctttttgaacacccagctccagtccaagacccaaggattccagctccaggaaccaggaggggcagtgatggggtcgatgatttggtttggctgagagtcccagcatttgtgttatggggaggatctctcatcttagagaagataaggggacagggcattcaggcaaggcagcttggggtttggtcaggagataagcgcccccttcccttggggggaggataagtgggggatggtcaacgttggagaagagtcaaagttctcaccacctttctaactactcagctaaactcgttgaggctagggccaacgtgacttctctgtagagaggatacaagccgggcctgatggggcaggcctgtgtaatcccagtcatagtggaggctgaggctggaaaattaaggaccaacagcctgggcaatttagtgtctcaaaataaaatgtaaagagggctgggaatgtagctcagtggtagggtgtttgtgtgaggctctgggatcaataagacaaaacaaccaaccaaccaaaaaccttccaaacaacaaaaccaaccctcaaaccaaaaaactatgtgggtgtctctgagtctggtttgaagagaacccgcagccctgtatccctgtggggctgtggacagtgggcagaagcagaggctccctggatcctgaacaagggccccaaaagcaagttctaaagggacccctgaaaccgagtaagcctttgtgtcaagaagtgggagtacaaccagaaaggtggctgagtgctttagag

SEQ ID NO: 29 - protein, 380 aa, Artificial Sequence
  Description of Artificial Sequence/note = synthetic construct
MDSPIQIFRGEPGPTCAPSACLPPNSSAWFPGWAEPDSNGSAGSEDAQLEPAHISPAIPVIITAVYSVVFVVGLVGNSLVMFVIIRYTKMKTATNIYIFNLALADALVTTTMPFQSTVYLMNSWPFGDVLCKIVISIDYYNMFTSIFTLTMMSVDRYIAVCHPVKALDFRTPLKAKIINICIWLLSSSVGISAIVLGGTKVREDVDVIECSLQFPDDDYSWWDLFMKICVFIFAFVIPVLIIIVCYTLMILRLKSVRLLSGSREKDRNLRRITRLVLVVVAVFVVCWTPIHIFILVEALGSTSHSTAALSSYYFCIALGYTNSSLNPILYAFLDENFKRCFRDFCFPLKMRMERQSTSRVRNTVQDPAYLRDIDGMNKPV

SEQ ID NO: 30 - DNA, 1154 bp, Artificial Sequence
  Description of Artificial Sequence/note = synthetic construct
atggactccccgatccagatcttccgcggggagccgggccctacctgcgccccgagcgcctgcctgccccccaacagcagcgcctggtttcccggctgggccgagcccgacagcaacggcagcgccggctcggaggacgcgcagctggagcccgcgcacatctccccggccatcccggtcatcatcacggcggtctactccgtagtgttcgtcgtgggcttggtgggcaactcgctggtcatgttcgtgatcatccgatacacaaagatgaagacagcaaccaacatttacatatttaacctggctttggcagatgctttagttactacaaccatgccctttcagagtacggtctacttgatgaattcctggccttttggggatgtgctgtgcaagatagtaatttccattgattactacaacatgttcaccagcatcttcaccttgaccatgatgagcgtggaccgctacattgccgtgtgccaccccgtgaaggctttggacttccgcacacccttgaaggcaaagatcatcaatatctgcatctggctgctgtcgtcatctgttggcatctctgcaatagtccttggaggcaccaaagtcagggaagacgtcgatgtcattgagtgctccttgcagttcccagatgatgactactcctggtgggacctcttcatgaagatctgcgtcttcatctttgccttcgtgatccctgtcctcatcatcatcgtctgctacaccctgatgatcctgcgtctcaagagcgtccggctcctttctggctcccgagagaaagatcgcaacctgcgtaggatcaccagactggtcctggtggtggtggcagtcttcgtcgtctgctggactcccattcacatattcatcctggtggaggctctggggagcacctcccacagcacagctgctctctccagctattacttctgcatcgccttaggctataccaacagtagcctgaatcccattctctacgcctttcttgatgaaaacttcaagcggtgtttccgggacttctgctttccactgaagatgaggatggagcggcagagcactagcagagtccgaaatacagttcaggatcctgcttacctgagggacatcgatgggatgaataaaccagtatgactagtcgtgga

SEQ ID NO: 31 - protein, 380 aa, Artificial Sequence
  Description of Artificial Sequence/note = synthetic construct
MESPIQIFRGDPGPTCSPSACLLPNSSSWFPNWAESDSNGSVGSEDQQLESAHISPAIPVIITAVYSVVFVVGLVGNSLVMFVIIRYTKMKTATNIYIFNLALADALVTTTMPFQSAVYLMNSWPFGDVLCKIVISIDYYNMFTSIFTLTMMSVDRYIAVCHPVKALDFRTPLKAKIINICIWLLASSVGISAIVLGGTKVREDVDVIECSLQFPDDEYSWWDLFMKICVFVFAFVIPVLIIIVCYTLMILRLKSVRLLSGSREKDRNLRRITKLVLVVVAVFIICWTPIHIFILVEALGSTSHSTAALSSYYFCIALGYTNSSLNPVLYAFLDENFKRCFRDFCFPIKMRMERQSTNRVRNTVQDPASMRDVGGMNKPV

SEQ ID NO: 32 - DNA, 1410 bp, Artificial Sequence
  Description of Artificial Sequence/note = synthetic construct
gcgcaccttgctgatcccaaacaggcagagcttcttccagtcttggaaggcacaaattgagcatcaggaacgtggacccatcagggctgaacagctactcaggatctaaagtggtgacttggaaagctgacggtgacttgggaagggaggtcgccaatcagcgatctggagctgcagcgctcaccatggagtcccccattcagatcttccgaggagatccaggccctacctgctctcccagtgcttgccttctccccaacagcagctcttggttccccaactgggcagaatccgacagtaatggcagtgtgggctcagaggatcagcagctggagtccgcgcacatctctccggccatccctgttatcatcaccgctgtctactctgtggtatttgtggtgggcttagtgggcaattctctggtcatgtttgtcatcatccgatacacgaagatgaagaccgcaaccaacatctacatatttaacctggctttggcagatgctttggttactaccactatgccctttcagagtgctgtctacttgatgaattcttggccttttggagatgtgctatgcaagattgtcatttccattgactactacaacatgtttaccagcatattcaccttgaccatgatgagtgtggaccgctacattgctgtgtgccaccctgtgaaagctttggacttccgaacacctttgaaagcaaagatcatcaacatctgcatttggctcctggcatcatctgttggtatatcagcgatagtccttggaggcaccaaagtcagggaagatgtggatgtcattgaatgctccttgcagtttcctgatgatgaatattcctggtgggatctcttcatgaagatctgtgtcttcgtctttgcctttgtgatcccagtcctcatcatcattgtctgctacaccctgatgatcctgcgcctgaagagtgtccggctcctgtctggctcccgagagaaggaccgaaatctccgccgcatcaccaagctggtgctggtagtagttgcagtcttcatcatctgttggacccccattcacatctttatcctggtggaggctctgggaagcacctcccacagcacagctgccctctccagctattatttctgtattgccttgggttataccaacagcagcctgaatcctgttctctatgcctttctggatgaaaacttcaagcggtgttttagggacttctgcttccctattaagatgcgaatggagcgccagagcaccaatagagttagaaacacagttcaggatcctgcttccatgagagatgtgggagggatgaataagccagtatgactagtcgtggaaatgtcttcttattgttctccaggtagagaagagttcaatgatcttggtttaacccagattacaactgcag

SEQ ID NO: 33 - protein, 380 aa, Artificial Sequence
  Description of Artificial Sequence/note = synthetic construct
MESPIQIFRGEPGPTCAPSACLLPNSSSWFPNWAESDSNGSVGSEDQQLEPAHISPAIPVIITAVYSVVFVVGLVGNSLVMFVIIRYTKMKTATNIYIFNLALADALVTTTMPFQSAVYLMNSWPFGDVLCKIVISIDYYNMFTSIFTLTMMSVDRYIAVCHPVKALDFRTPLKAKIINICIWLLASSVGISAIVLGGTKVREDVDVIECSLQFPDDEYSWWDLFMKICVFVFAFVIPVLIIIVCYTLMILRLKSVRLLSGSREKDRNLRRITKLVLVVVAVFIICWTPIHIFILVEALGSTSHSTAVLSSYYFCIALGYTNSSLNPVLYAFLDENFKRCFRDFYFPIKMRMERQSTNRVRNTVQDPASMRDVGGMNKPV

SEQ ID NO: 34 - DNA, 2481 bp, Artificial Sequence
  Description of Artificial Sequence/note = synthetic construct
tccagccctgcctgcacaggcaaagtttgtctctctgggtccaatctgtcctctgctcctgcagcccggcaagtgccatcgctcctcggtttccagctgcagcactcaccatggagtcccccatccagattttccgcggagagccaggccctacctgtgctcccagtgcttgcctactccccaacagcagctcttggttccccaactgggccgaatcggacagcaatggcagtttgggctccgaggaccagcagctggagcccgcgcacatctctccagccatccctgttatcatcaccgctgtctactctgtggtgtttgtggtgggcttagtgggcaattccctggtcatgtttgtcatcatccgatacacaaagatgaagaccgcaaccaacatctacatatttaacctggctttggcagatgctttggttactaccactatgcccttccagagtgctgtctacttgatgaattcttggccttttggagatgttctgtgcaagattgtcatttccattgactactacaacatgtttaccagcatattcaccttgaccatgatgagtgtggaccgctacattgccgtgtgccaccctgtgaaagctttggatttccgaacacctttgaaagcaaagatcatcaacatctgcatttggctactggcatcatctgttggtatatcagcgatagtccttggaggcaccaaagtcagggaagatgtggatgtcattgaatgctccttgcagtttcctgatgatgaatattcctggtgggacctcttcatgaagatctgtgtcttcgtctttgcctttgttatccctgtcttaatcatcattgtctgctacaccctgatgatcctgcgcttgaagagtgtccggctcctctcgggctctcgagagaaggaccgaaatctccgccggatcaccaagctggtgctggtagtggttgcagtcttcatcatctgttggacccccatccacatctttatcctggtcgaggctctaggcagcacctctcacagcacagctgtcctctctagctattacttctgcattgccttgggttataccaacagcagcttgaatcctgttctctatgcctttcttgatgaaaacttcaagcggtgttttagggacttctgcttccccattaagatgcgaatggagcgccagagcacaaacagagttagaaacacagttcaggatcctgcttccatgagggatgtgggtgggatgaataagccagtatgactagtcatggaaatgtcttcctattgttctccgggtagagaagagttcaatgatcttggtttaacccagattaccactgcagtctgaagaggaaagatgaggtattcaataacttagccatgttatgcaatctaaaggtgcagggcacattagtgatctaggctgagtaggggcagcaagtgtgaagaacagagcacatgtcctggcaacaatacacctctttcctaggacagaggagaaggcaatctaacctcaacccttcgataaacagacagcactctttcttctggtccccttgatttactgcacctccatctgcgtggcctttctgtgtaacatagttccaaagctctagagaagaaaatgaaagaaaaagtgcatttgatccaaaacttactgggcacccaaccttcgcgttaacacaggcaaaaccaacttcgtatacaatgaggccataccaagggtcatatccaactcactttctgctggtgatcatgtcttatgtgtgatgagagtgaaacttaggaagaacctggactcagaccctgacactgggggagagcaccatattgacatttgtgaacctatttaaagttgtgggtgttctcgtctcacagtgtctaatgccttgaaaaactacagttgcttcttaaggtctctggtttttagcatgctattcaggaagataatcttctgagaaaacatgaactgatattaaaaggttgaagcttaataccagcaaagtgtgtgtaatttcatctgtaaatagtggtctgtatataaataaggaccaggttttcctgtccagcctgtacatttctcaaggatgccgtagacacacccctggaggcatggaaagttcatgctgggatattttgcttcactataagctactttcttgatttggtcttggtgtgatttctactagattactcaaacattatttactctaacactgatcataacttggtgttaacaattccccaaactttgaattcattctaaagtgttagcattgatcaaatctactttgtggtagcatctgtttgtaaacacacacatattgccagattctctactcaggtagaggaagttgctttgatcatgtacaccttcaaatgttatgctctggctttccacagaaagtggaattgtttcaaaatgcatgctgaaaaaggaaataggatttgagatggcttagcacaatttgcatggtattgagtaagag

SEQ ID NO: 35 - DNA, 21 bp, Artificial Sequence
  Description of Artificial Sequence/note = synthetic construct
tttttccagttccgtttatcc

SEQ ID NO: 36 - DNA, 20 bp, Artificial Sequence
  Description of Artificial Sequence/note = synthetic construct
tttatcgccaatccacatct

SEQ ID NO: 37 - DNA, 20 bp, Artificial Sequence
  Description of Artificial Sequence/note = synthetic construct
cccatagtaacgccaatagg

SEQ ID NO: 38 - DNA, 21 bp, Artificial Sequence
  Description of Artificial Sequence/note = synthetic construct
aaatgtgagcgagtaacaacc

SEQ ID NO: 39 - DNA, 20 bp, Artificial Sequence
  Description of Artificial Sequence/note = synthetic construct
accacagtccatgccatcac

SEQ ID NO: 40 - DNA, 20 bp, Artificial Sequence
  Description of Artificial Sequence/note = synthetic construct
tccaccaccctgttgctgta

SEQ ID NO: 41 - DNA, 23 bp, Artificial Sequence
  Description of Artificial Sequence/note = synthetic construct
cacctaatacgactcactatagg

SEQ ID NO: 42 - DNA, 18 bp, Artificial Sequence
  Description of Artificial Sequence/note = synthetic construct
cattaaccctcactaaag

SEQ ID NO: 43 - DNA, 35 bp, Artificial Sequence
  Description of Artificial Sequence/note = synthetic construct
atatcgatatcgctagcttttaaaagaaaaggggg

SEQ ID NO: 44 - DNA, 39 bp, Artificial Sequence
  Description of Artificial Sequence/note = synthetic construct
taatcgatgctaagcaaaattttgaatttttgtaatttg

SEQ ID NO: 45 - DNA, 22 bp, Artificial Sequence
  Description of Artificial Sequence/note = synthetic construct
gaattacctaatgggaacatgg

SEQ ID NO: 46 - DNA, 18 bp, Artificial Sequence
  Description of Artificial Sequence/note = synthetic construct
gcagacgatgaacacagc

SEQ ID NO: 47 - DNA, 14 bp, Artificial Sequence
  Description of Artificial Sequence/note = synthetic construct
accacagcaatcac

SEQ ID NO: 48 - DNA, 10472 bp, Artificial Sequence
  Description of Artificial Sequence/note = synthetic construct
aatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgataagcttgggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgactctagaggatccactagtccagtgtggtggaattgatcccttcacctaatacgactcactataggctagctcctcctctgctcgcccaatccttccaaccccctatggtggtatggctgacacagaaaatgtctgctcctgtatgggacatttgcccctcttctccaaatataagacaggatgaggcctagcttttgctgctccaaagttttaaaagaacacattgcacggcatttagggactctaaagggtggaggaggaatgagggaattgcatcatgccaaggctggtcctcatccatcactgcttccagggcccagagtggcttccaggaggtattcttacaaaggaagcccgatctgtagctaacactcagagcccattttcctgcgttaacccctcccgacctcatatacaggagtaacatgatcagtgacctgggggagctggccaaactgcgggacctgcccaagctgagggccttggtgctgctggacaacccctgtgccgatgagactgactaccgccaggaggccctggtgcagatggcacacctagagcgcctagacaaagagtactatgaggacgaggaccgggcagaagctgaggagatccgacagaggctgaaggaggaacaggagcaagaactcgacccggaccaagacatggaaccgtacctcccgccaacttagtggctcctctagcctgcagggacagtaaaggtgatggcaggaaggcagcccccggaggtcaaaggctgggcacgcgggaggagaggccagagtcagaggctgcgggtatctcagatatgaaggaaagatgagagaggctcaggaagaggtaagaaaagacacaagagaccagagaagggagaagaattagagagggaggcagaggaccgctgtctctacagacatagctggtagagactgggaggaagggatgaaccctgagcgcatgaagggaaggaggtggctggtggtatatggaggatgtagctgggccagggaaaagatcctgcactaaaaatctgaagctaaaaataacaggacacggggtggagaggcgaaaggagggcagattgaggcagagagactgagaggcctggggatgtgggcattccggtagggcacacagttcacttgtcttctctttttccaggaggccaragatgctgacctcaagaactcataataccccagtggggaccaccgcattcatagccctgttacaagaagtgggagatgttcctttttgtcccagactggaaatccattacatcccgaggctcaggttctgtggtggtcatctctgtgtggcttgttctgtgggcctacctaaagtcctaagcacagctctcaagcagatccgaggcgactaagatgctagtaggggttgtctggagagaagagccgaggaggtgggctgtgatggatcagttcagctttcaaataaaaaggcgtttttatattctgtgtcgagttcgtgaacccctgtggtgggcttctccatctgtctgggttagtacctgccactatactggaataaggggacgcctgcttccctcgagttggctggacaaggttatgagcatccgtgtacttatggggttgccagcttggtcctggatcgcccgggcccttcccccacccgttcggttccccaccaccacccgcgctcgtacgtgcgtctccgcctgcagctcttgactcatcggggcccccgggtcacatgcgctcgctcggctctataggcgccgccccctgcccaccccccgcccgcgctgggagccgcagccgccgccactcctgctctctctgcgccgccgccgtcaccaccgccaccgccaccggctgagtctgcagtcctcgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggccaagctaagcttgatatcgaattccggatgagcctctgtgaactactaaggtgggagggggctatacgcagaggagaatgtcagatgctcagctcggtcccctccgcctgacgctcctctctgtctcagccaggactggtttctgtaagaaacagcaggagctgtggcagcggcgaaaggaagcggctgaggcgcttggaacccgaaaagtctcggtgctcctggctacctcgcacagcggtgcccgcccggccgtcagtaccatggacagcagcgctgcccccacgaacgccagcaattgcactgatgccttggcgtactcaagttgctccccagcacccagccccggttcctgggtcaacttgtcccacttagatggcaacctgtccgacccatgcggtccgaaccgcaccgacctgggcgggagagacagcctgtgccctccgaccggcagtccctccatgatcacggccatcacgatcatggccctctactccatcgtgtgcgtggtggggctcttcggaaacttcctggtcatgtatgtgattgtcagatacaccaagatgaagactgccaccaacatctacattttcaaccttgctctggcagatgccttagccaccagtaccctgcccttccagagtgtgaattacctaatgggaacatggccatttggaaccatcctttgcaagatagtgatctccatagattactataacatgttcaccagcatattcaccctctgcaccatgagtgttgatcgatacattgcagtctgccaccctgtcaaggccttagatttccgtactccccgaaatgccaaaattatcaatgtctgcaactggatcctctcttcagccattggtcttcctgtaatgttcatggctacaacaaaatacaggcaaggttccatagattgtacactaacattctctcatccaacctggtactgggaaaacctgctgaagatctgtgttttcatcttcgccttcattatgccagtgctcatcattaccgtgtgctatggactgatgatcttgcgcctcaagagtgtccgcatgctctctggctccaaagaaaaggacaggaatcttcgaaggatcaccaggatggtgctggtggtggtggctgtgttcatcgtctgctggactcccattcacatttacgtcatcattaaagccttggttacaatcccagaaactacgttccagactgtttcttggcacttctgcattgctctaggttacacaaacagctgcctcaacccagtcctttatgcatttctggatgaaaacttcaaacgatgcttcagagagttctgtatcccaacctcttccaacattgagcaacaaaactccactcgaattcgtcagaacatctagagaccacccctccacggccaatacagtggatagaactaatcatcagctagaaaatctggaagcagaaactgctccgttgcccgtcgacccgggcggccgcttccctttagtgagggttaatgaagggctcgagtctagagggcccgcggttcgaaggtaagcctatccctaaccctctcctcggtctcgattctacgcgtaccggttagtaatgagtttggaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagcctttgtctcaagaagaatccaccctcattgaaagagcaacggctacaatcaacagcatccccatctctgaagactacagcgtcgccagcgcagctctctctagcgacggccgcatcttcactggtgtcaatgtatatcattttactgggggaccttgtgcagaactcgtggtgctgggcactgctgctgctgcggcagctggcaacctgacttgtatcgtcgcgatcggaaatgagaacaggggcatcttgagcccctgcggacggtgccgacaggtgcttctcgatctgcatcctgggatcaaagccatagtgaaggacagtgatggacagccgacggcagttgggattcgtgaattgctgccctctggttatgtgtgggagggctaagcacaattcgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagctt

SEQ ID NO: 49 - DNA, 8889 bp, Artificial Sequence
  Description of Artificial Sequence/note = synthetic construct
ttggcccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctcccctcgaagcttacatgtggtaccgagctcggatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagcacgtgagatctgaattcgagatctgccgccgccatgggtgcgagagcgtcagtattaagcgggggagaattagatcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaaaaaagcacagcaagcagcagctgacacaggacacagcaatcaggtcagccaaaattaccctatagtgcagaacatccaggggcaaatggtacatcaggccatatcacctagaactttaaatgcatgggtaaaagtagtagaagagaaggctttcagcccagaagtgatacccatgttttcagcattatcagaaggagccaccccacaagatttaaacaccatgctaaacacagtggggggacatcaagcagccatgcaaatgttaaaagagaccatcaatgaggaagctgcagaatgggatagagtgcatccagtgcatgcagggcctattgcaccaggccagatgagagaaccaaggggaagtgacatagcaggaactactagtacccttcaggaacaaataggatggatgacacataatccacctatcccagtaggagaaatctataaaagatggataatcctgggattaaataaaatagtaagaatgtatagccctaccagcattctggacataagacaaggaccaaaggaaccctttagagactatgtagaccgattctataaaactctaagagccgagcaagcttcacaagaggtaaaaaattggatgacagaaaccttgttggtccaaaatgcgaacccagattgtaagactattttaaaagcattgggaccaggagcgacactagaagaaatgatgacagcatgtcagggagtggggggacccggccataaagcaagagttttggctgaagcaatgagccaagtaacaaatccagctaccataatgatacagaaaggcaattttaggaaccaaagaaagactgttaagtgtttcaattgtggcaaagaagggcacatagccaaaaattgcagggcccctaggaaaaagggctgttggaaatgtggaaaggaaggacaccaaatgaaagattgtactgagagacaggctaattttttagggaagatctggccttcccacaagggaaggccagggaattttcttcagagcagaccagagccaacagccccaccagaagagagcttcaggtttggggaagagacaacaactccctctcagaagcaggagccgatagacaaggaactgtatcctttagcttccctcagatcactctttggcagcgacccctcgtcacaataaagataggggggcaattaaaggaagctctattagatacaggagcagatgatacagtattagaagaaatgaatttgccaggaagatggaaaccaaaaatgatagggggaattggaggttttatcaaagtaagacagtatgatcagatactcatagaaatctgcggacataaagctataggtacagtattagtaggacctacacctgtcaacataattggaagaaatctgttgactcagattggctgcactttaaattttcccattagtcctattgagactgtaccagtaaaattaaagccaggaatggatggcccaaaagttaaacaatggccattgacagaagaaaaaataaaagcattagtagaaatttgtacagaaatggaaaaggaaggaaaaatttcaaaaattgggcctgaaaatccatacaatactccagtatttgccataaagaaaaaagacagtactaaatggagaaaattagtagatttcagagaacttaataagagaactcaagatttctgggaagttcaattaggaataccacatcctgcagggttaaaacagaaaaaatcagtaacagtactggatgtgggcgatgcatatttttcagttcccttagataaagacttcaggaagtatactgcatttaccatacctagtataaacaatgagacaccagggattagatatcagtacaatgtgcttccacagggatggaaaggatcaccagcaatattccagtgtagcatgacaaaaatcttagagccttttagaaaacaaaatccagacatagtcatctatcaatacatggatgatttgtatgtaggatctgacttagaaatagggcagcatagaacaaaaatagaggaactgagacaacatctgttgaggtggggatttaccacaccagacaaaaaacatcagaaagaacctccattcctttggatgggttatgaactccatcctgataaatggacagtacagcctatagtgctgccagaaaaggacagctggactgtcaatgacatacagaaattagtgggaaaattgaattgggcaagtcagatttatgcagggattaaagtaaggcaattatgtaaacttcttaggggaaccaaagcactaacagaagtagtaccactaacagaagaagcagagctagaactggcagaaaacagggagattctaaaagaaccggtacatggagtgtattatgacccatcaaaagacttaatagcagaaatacagaagcaggggcaaggccaatggacatatcaaatttatcaagagccatttaaaaatctgaaaacaggaaagtatgcaagaatgaagggtgcccacactaatgatgtgaaacaattaacagaggcagtacaaaaaatagccacagaaagcatagtaatatggggaaagactcctaaatttaaattacccatacaaaaggaaacatgggaagcatggtggacagagtattggcaagccacctggattcctgagtgggagtttgtcaatacccctcccttagtgaagttatggtaccagttagagaaagaacccataataggagcagaaactttctatgtagatggggcagccaatagggaaactaaattaggaaaagcaggatatgtaactgacagaggaagacaaaaagttgtccccctaacggacacaacaaatcagaagactgagttacaagcaattcatctagctttgcaggattcgggattagaagtaaacatagtgacagactcacaatatgcattgggaatcattcaagcacaaccagataagagtgaatcagagttagtcagtcaaataatagagcagttaataaaaaaggaaaaagtctacctggcatgggtaccagcacacaaaggaattggaggaaatgaacaagtagataaattggtcagtgctggaatcaggaaagtactatttttagatggaatagataaggcccaagaagaacatgagaaatatcacagtaattggagagcaatggctagtgattttaacctaccacctgtagtagcaaaagaaatagtagccagctgtgataaatgtcagctaaaaggggaagccatgcatggacaagtagactgtagcccaggaatatggcagctagattgtacacatttagaaggaaaagttatcttggtagcagttcatgtagccagtggatatatagaagcagaagtaattccagcagagacagggcaagaaacagcatacttcctcttaaaattagcaggaagatggccagtaaaaacagtacatacagacaatggcagcaatttcaccagtactacagttaaggccgcctgttggtgggcggggatcaagcaggaatttggcattccctacaatccccaaagtcaaggagtaatagaatctatgaataaagaattaaagaaaattataggacaggtaagagatcaggctgaacatcttaagacagcagtacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggaaaggaccagcaaagctcctctggaaaggtgaaggggcagtagtaatacaagataatagtgacataaaagtagtgccaagaagaaaagcaaagatcatcagggattatggaaaacagatggcaggtgatgattgtgtggcaagtagacaggatgaggattaacacatggaattccggagcggccgcaggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttccgcggaattcaccccaccagtgcaggctgcctatcagaaagtggtggctggtgtggctaatgccctggcccacaagtatcactaagctcgctttcttgctgtccaatttctattaaaggttcctttgttccctaagtccaactactaaactgggggatattatgaagggccttgagcatctggattctgcctaataaaaaacatttattttcattgcaatgatgtatttaaattatttctgaatattttactaaaaagggaatgtgggaggtcagtgcatttaaaacataaagaaatgaagagctagttcaaaccttgggaaaatacactatatcttaaactccatgaaagaaggtgaggctgcaaacagctaatgcacattggcaacagcccctgatgcctatgccttattcatccctcagaaaaggattcaagtagaggcttgatttggaggttaaagttttgctatgctgtattttacattacttattgttttagctgtcctcatgaatgtcttttcactacccatttgcttatcctgcatctctcagccttgactccactcagttctcttgcttagagataccacctttcccctgaagtgttccttccatgttttacggcgagatggtttctcctcgcctggccactcagccttagttgtctctgttgtcttatagaggtctacttgaagaaggaaaaacagggggcatggtttgactgtcctgtgagcccttcttccctgcctcccccactcacagtgacccggaatccctcgacatggcagtctagcactagtgcggccgcagatctgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgggatcccctgagggggcccccatgggctagaggatccggcctcggcctctgcataaataaaaaaaattagtcagccatgagc

SEQ ID NO: 50 - DNA, 4180 bp, Artificial Sequence
  Description of Artificial Sequence/note = synthetic construct
aatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattccgcattgcagagatattgtatttaagtgcctagctcgatacaataaacgccatttgaccattcaccacattggtgtgcacctccaagctcgagctcgtttagtgaaccgtcagatcgcctggagacgccatccacgctgttttgacctccatagaagacaccgggaccgatccagcctcccctcgaagctagtcgattaggcatctcctatggcaggaagaagcggagacagcgacgaagacctcctcaaggcagtcagactcatcaagtttctctatcaaagcaacccacctcccaatcccgaggggacccgacaggcccgaaggaatagaagaagaaggtggagagagagacagagacagatccattcgattagtgaacggatccttagcacttatctgggacgatctgcggagcctgtgcctcttcagctaccaccgcttgagagacttactcttgattgtaacgaggattgtggaacttctgggacgcagggggtgggaagccctcaaatattggtggaatctcctacaatattggagtcaggagctaaagaatagtgctgttagcttgctcaatgccacagctatagcagtagctgaggggacagatagggttatagaagtagtacaagaagcttggcactggccgtcgttttacaacgtcgtgatctgagcctgggagatctctggctaactagggaacccactgcttaagcctcaataaagcttgccttgagtgcttcaagtagtgtgtgcccgtctgttgtgtgactctggtaactagagatcaggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgacatgattacgaattcgatgtacgggccagatatacgcgtatctgaggggactagggtgtgtttaggcgaaaagcggggcttcggttgtacgcggttaggagtcccctcaggatatagtagtttcgcttttgcatagggaggggga

SEQ ID NO: 51 - DNA, 5821 bp, Artificial Sequence
  Description of Artificial Sequence/note = synthetic construct
ttggcccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctcccctcgaagcttacatgtggtaccgagctcggatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagcacgtgagatctgaattctgacactatgaagtgccttttgtacttagcctttttattcattggggtgaattgcaagttcaccatagtttttccacacaaccaaaaaggaaactggaaaaatgttccttctaattaccattattgcccgtcaagctcagatttaaattggcataatgacttaataggcacagccttacaagtcaaaatgcccaagagtcacaaggctattcaagcagacggttggatgtgtcatgcttccaaatgggtcactacttgtgatttccgctggtatggaccgaagtatataacacattccatccgatccttcactccatctgtagaacaatgcaaggaaagcattgaacaaacgaaacaaggaacttggctgaatccaggcttccctcctcaaagttgtggatatgcaactgtgacggatgccgaagcagtgattgtccaggtgactcctcaccatgtgctggttgatgaatacacaggagaatgggttgattcacagttcatcaacggaaaatgcagcaattacatatgccccactgtccataactctacaacctggcattctgactataaggtcaaagggctatgtgattctaacctcatttccatggacatcaccttcttctcagaggacggagagctatcatccctgggaaaggagggcacagggttcagaagtaactactttgcttatgaaactggaggcaaggcctgcaaaatgcaatactgcaagcattggggagtcagactcccatcaggtgtctggttcgagatggctgataaggatctctttgctgcagccagattccctgaatgcccagaagggtcaagtatctctgctccatctcagacctcagtggatgtaagtctaattcaggacgttgagaggatcttggattattccctctgccaagaaacctggagcaaaatcagagcgggtcttccaatctctccagtggatctcagctatcttgctcctaaaaacccaggaaccggtcctgctttcaccataatcaatggtaccctaaaatactttgagaccagatacatcagagtcgatattgctgctccaatcctctcaagaatggtcggaatgatcagtggaactaccacagaaagggaactgtgggatgactgggcaccatatgaagacgtggaaattggacccaatggagttctgaggaccagttcaggatataagtttcctttatacatgattggacatggtatgttggactccgatcttcatcttagctcaaaggctcaggtgttcgaacatcctcacattcaagacgctgcttcgcaacttcctgatgatgagagtttattttttggtgatactgggctatccaaaaatccaatcgagcttgtagaaggttggttcagtagttggaaaagctctattgcctcttttttctttatcatagggttaatcattggactattcttggttctccgagttggtatccatctttgcattaaattaaagcacaccaagaaaagacagatttatacagacatagagatgaaccgacttggaaagtaactcaaatcctgcacaacagattcttcatgtttggaccaaatcaacttgtgataccatgctcaaagaggcctcaattatatttgagtttttaatttttatgaaaaaaaaaaaaaaaaacggaattcaccccaccagtgcaggctgcctatcagaaagtggtggctggtgtggctaatgccctggcccacaagtatcactaagctcgctttcttgctgtccaatttctattaaaggttcctttgttccctaagtccaactactaaactgggggatattatgaagggccttgagcatctggattctgcctaataaaaaacatttattttcattgcaatgatgtatttaaattatttctgaatattttactaaaaagggaatgtgggaggtcagtgcatttaaaacataaagaaatgaagagctagttcaaaccttgggaaaatacactatatcttaaactccatgaaagaaggtgaggctgcaaacagctaatgcacattggcaacagcccctgatgcctatgccttattcatccctcagaaaaggattcaagtagaggcttgatttggaggttaaagttttgctatgctgtattttacattacttattgttttagctgtcctcatgaatgtcttttcactacccatttgcttatcctgcatctctcagccttgactccactcagttctcttgcttagagataccacctttcccctgaagtgttccttccatgttttacggcgagatggtttctcctcgcctggccactcagccttagttgtctctgttgtcttatagaggtctacttgaagaaggaaaaacagggggcatggtttgactgtcctgtgagcccttcttccctgcctcccccactcacagtgacccggaatccctcgacatggcagtctagcactagtgcggccgcagatctgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgggatcccctgagggggcccccatgggctagaggatccggcctcggcctctgcataaataaaaaaaattagtcagccatgagc

SEQ ID NO: 52 - DNA, 1986 bp, Artificial Sequence; identical to SEQ ID NO: 11
  Description of Artificial Sequence/note = synthetic construct

SEQ ID NO: 53 - DNA, 1610 bp, Artificial Sequence
  Description of Artificial Sequence/note = synthetic construct
cggatgagcctctgtgaactactaaggtgggagggggctatacgcagaggagaatgtcagatgctcagctcggtcccctccgcctgacgctcctctctgtctcagccaggactggtttctgtaagaaacagcaggagctgtggcagcggcgaaaggaagcggctgaggcgcttggaacccgaaaagtctcggtgctcctggctacctcgcacagcgtgcccgcccggccgtcagtaccatggacagcagcgctgcccccacgaacgccagcaattgcactgatgccttggcgtactcaagttgctccccagcacccagccccggttcctgggtcaacttgtcccacttagatggcaacctgtccgacccatgcggtccgaaccgcaccgacctgggcgggagagacagcctgtgccctccgaccggcagtccctccatgatcacggccatcacgatcatggccctctactccatcgtgtgcgtggtggggctcttcggaaacttcctggtcatgtatgtgattgtcagatacaccaagatgaagactgccaccaacatctacattttcaaccttgctctggcagatgccttagccaccagtaccctgcccttccagagtgtgaattacctaatgggaacatggccatttggaaccatcctttgcaagatagtgatctccatagattactataacatgttcaccagcatattcaccctctgcaccatgagtgttgatcgatacattgcagtctgccaccctgtcaaggccttagatttccgtactccccgaaatgccaaaattatcaatgtctgcaactggatcctctcttcagccattggtcttcctgtaatgttcatggctacaacaaaatacaggcaaggttccatagattgtacactaacattctctcatccaacctggtactgggaaaacctgctgaagatctgtgttttcatcttcgccttcattatgccagtgctcatcattaccgtgtgctatggactgatgatcttgcgcctcaagagtgtccgcatgctctctggctccaaagaaaaggacaggaatcttcgaaggatcaccaggatggtgctggtggtggtggctgtgttcatcgtctgctggactcccattcacatttacgtcatcattaaagccttggttacaatcccagaaactacgttccagactgtttcttggcacttctgcattgctctaggttacacaaacagctgcctcaacccagtcctttatgcatttctggatgaaaacttcaaacgatgcttcagagagttctgtatcccaacctcttccaacattgagcaacaaaactccactcgaattcgtcagaacactagagaccacccctccacggccaatacagtggatagaactaatcatcagctagaaaatctggaagcagaaactgctccgttgccctaacagggtctcatgccattccgaccttcaccaagcttagaagccaccatgtatgtggaagcaggttgcttcaagaatgtgtaggaggctctaattctctaggaaagtgcctgcttttaggtcatccaacctctttcctctctggccactctgctctgcacattagaggccg

SEQ ID NO: 54 - DNA, 1536 bp, Artificial Sequence
  Description of Artificial Sequence/note = synthetic construct
atgaagtgccttttgtacttagcctttttattcattggggtgaattgcaagttcaccatagtttttccacacaaccaaaaaggaaactggaaaaatgttccttctaattaccattattgcccgtcaagctcagatttaaattggcataatgacttaataggcacagccttacaagtcaaaatgcccaagagtcacaaggctattcaagcagacggttggatgtgtcatgcttccaaatgggtcactacttgtgatttccgctggtatggaccgaagtatataacacattccatccgatccttcactccatctgtagaacaatgcaaggaaagcattgaacaaacgaaacaaggaacttggctgaatccaggcttccctcctcaaagttgtggatatgcaactgtgacggatgccgaagcagtgattgtccaggtgactcctcaccatgtgctggttgatgaatacacaggagaatgggttgattcacagttcatcaacggaaaatgcagcaattacatatgccccactgtccataactctacaacctggcattctgactataaggtcaaagggctatgtgattctaacctcatttccatggacatcaccttcttctcagaggacggagagctatcatccctgggaaaggagggcacagggttcagaagtaactactttgcttatgaaactggaggcaaggcctgcaaaatgcaatactgcaagcattggggagtcagactcccatcaggtgtctggttcgagatggctgataaggatctctttgctgcagccagattccctgaatgcccagaagggtcaagtatctctgctccatctcagacctcagtggatgtaagtctaattcaggacgttgagaggatcttggattattccctctgccaagaaacctggagcaaaatcagagcgggtcttccaatctctccagtggatctcagctatcttgctcctaaaaacccaggaaccggtcctgctttcaccataatcaatggtaccctaaaatactttgagaccagatacatcagagtcgatattgctgctccaatcctctcaagaatggtcggaatgatcagtggaactaccacagaaagggaactgtgggatgactgggcaccatatgaagacgtggaaattggacccaatggagttctgaggaccagttcaggatataagtttcctttatacatgattggacatggtatgttggactccgatcttcatcttagctcaaaggctcaggtgttcgaacatcctcacattcaagacgctgcttcgcaacttcctgatgatgagagtttattttttggtgatactgggctatccaaaaatccaatcgagcttgtagaaggttggttcagtagttggaaaagctctattgcctcttttttctttatcatagggttaatcattggactattcttggttctccgagttggtatccatctttgcattaaattaaagcacaccaagaaaagacagatttatacagacatagagatgaaccgacttggaaagtaa

(19) United States
(12) Patent Application Publication
(10) Pub. No.: US 2007/0016968 A1
(43) Pub. Date: Jan. 18, 2007

(54) Treatment of pain through expression of opioid receptors

(76) Inventors: Stephanos Kyrkanides, Penfield, NY (US); Ross H. Tallents, Rochester, NY (US)

Correspondence Address: NEEDLE & ROSENBERG, P.C., SUITE 1000, 999 PEACHTREE STREET, ATLANTA, GA, 30309-3915, US

(21) Appl. No.: 10/546,179

(22) PCT Filed: Feb. 19, 2004
(86) PCT No.: PCT/US04/04914

§ 371 Date: Aug. 8, 2006

Related U.S. Application Data
(60) Provisional application No. 60/448,663, filed on Feb. 19, 2003.

(51) Int. Cl. A01K 67/027; A61K 48/00; C12N 15/867; C12N 5/06

(52) U.S. Cl. 800/14; 514/44; 435/455; 435/456; 435/354

(57) ABSTRACT

Disclosed are compositions and methods related to nucleic acid constructs containing a HUMOR encoding element. These constructs can be used in the treatment of pain.

This application claims priority of U.S. Provisional Application No. 60/448,663, filed on Feb. 19, 2003, which is herein incorporated by reference in its entirety.

### I. BACKGROUND

Tissue injury and nerve damage caused by trauma, infection, arthritis or iatrogenic procedures can produce inflammation, spontaneous pain and hyperalgesia (Levine J D and Taiwo Y O, Anesth Prog 1990; 37:133-35; Goelet P, et al., Nature. 1986; 322(6078):419-22). Furthermore, patients affected by temporomandibular joint and/or masticatory muscle (orofacial) pain often suffer because dental, surgical and/or pharmacologic therapies do not consistently give adequate symptom relief. In fact, according to Public Health Services (PHS) estimates, there are more than 50 million Americans who experience chronic pain with 45% seeking medical care at some point in their lives. It is also estimated that 40% of pain patients never receive adequate relief. Lipton et al. (Lipton J A, et al., JADA 1993; 124:115-21.) reported that 22% of the population in the United States experienced at least one episode of orofacial pain in the last six months. It is estimated that approximately $80 billion is spent annually to treat pain and that 40% of that is to treat craniofacial pain (Bonica J J. Preface et al., eds. Advances in Pain Research Therapy, Vol 3. New York: Raven Press; 1973:v-vii). To date, pharmacological approaches still dominate the clinical pain arena, with only modest efforts being directed towards the development of new innovative treatment regimes for the management of pain. Disclosed are vectors and methods for reducing pains, such as myalgic and arthralgic pains, and such as those in the orofacial region.

### II. SUMMARY

As embodied and broadly described herein, disclosed herein, in one aspect, are vector constructs that comprise sequence encoding a polypeptide for treating pain. Also disclosed are methods for treating pain by expressing the μ-opioid receptor protein in nerve cells.

It is to be understood that both the foregoing general description and the following detailed description are exemplary and explanatory only and are not restrictive.

### III. BRIEF DESCRIPTION OF THE DRAWINGS

FIG. 1 shows the human μ-opioid transient transfection in N2a cells. FIG. 1A shows that immunocytochemistry reveals expression of HUMOR driven by the cytomegalovirus CMV promoter in neuronal cells using monoclonal antibody. FIG. 1B shows mock transfected cells.

FIG. 2 shows FIV(lacZ) local administration to the temporomandibular joint (TMJ) area of the face. FIG. 2A shows FIV was injected at the right TMJ with 10⁸ infectious particles per mL, which receives sensory innervation from the mandibular division of the trigeminal nerve. Saggital (B) and horizontal (C) sections of the right gasserion (trigeminal) ganglion display X-gal positive neuronal cellbodies that were transduced following FIV(lacZ) injection in the TMJ.

FIG. 3 shows a representation of a lentiviral system containing the HUMOR gene. The 3-vector FIV(HUMOR) system. The FIV(HUMOR) lentiviral system is comprised of 3 vectors: Packaging vector providing the packaging instructions in trans,—VSV-G envelop vector (VSV-G sequence in SEQ ID NO:54) providing the envelop instructions in trans,—and FIV(HUMOR) vector containing the therapeutic gene.

FIG. 4 shows a schematic of an exemplary LUV vector carrying a HUMOR cassette.

FIG. 5 shows FIV(lacZ) injection (a total of 5×10⁶ infectious particles) to the right TMJ resulted in widespread infection of hard as well as soft tissues of the joint. (A) Sagittal TMJ sections analyzed by β-galactosidase immunohistochemistry and counter-stained by nuclear fast red revealed expression of the reporter gene lacZ in the hypertrophic zone of the condyle, primarily comprised of cartilaginous cells, (B) as well as in the meniscus, endothelial cells and perivascular osteocytes. Panel (C) depicts TMJ sections from a saline injected animal. c=condyle; d=disk; m=muscle; v—vessel.

FIG. 6 shows the development of the control FIV(Δ'lac) vector with inactive β-galactosidase gene. (A) The reporter gene lacZ was inactivated after deletion of a critical placZ DNA fragment containing the β-galactosidase gene transcription initiation site by restriction enzyme-mediated excision and re-ligation of the backbone vector. (B) The structure of mutated FIV(Δ'lac) and wild type FIV(lacZ) viral vectors were confirmed by PCR following transient transfection into the murine cell line NIH 3T3. The presence of viral DNA in cells was detected by a 444 bp DNA band utilizing the ‘FIV’ primers (as depicted in panel A). The complete structure of lacZ gene was confirmed by a 1.7 kb DNA band utilizing the lacZ primers (depicted as UP, LP in panel A). In the case of the mutated FIV(Δ'lac), there was lack of the 1.7 kb DNA band as the annealing site for the lower primer LP was excised. (C) Deletion of the lacZ transcription initiation sequence in the FIV(Δ'lac) resulted in inactivation of the β-galactosidase reporter gene as demonstrated by the lack of X-gal staining compared to (D) the FIV(lacZ) vector.

FIG. 7 shows FIV(lacZ) and FIV(Δ'lac) injections (5×10⁶ infectious particles) in the right TMJ of mice resulted in successful infection of primary sensory neurons located in the ispilateral trigeminal ganglion. The animals' left side TMJ was not treated (A) The presence of backbone FIV DNA in the right trigeminal ganglia ipsilateral to FIV injections was detected by a 444 bp DNA band in lanes 1 and 3, utilizing the “FIV” primers (as depicted in panel A), suggesting successful transduction of the trigeminal sensory neurons by FIV vectors. Lanes 2 and 4 do not display any viral DNA as they represent left side ganglia. (B) The inactive form of β-galactosidase gene in transduced neurons was detected by the absence of the 1.7 kb DNA band (lane 1) compared with the wild type gene (lane 3). Lanes 2 and 4 do not display any viral DNA as they represent left side ganglia. (C) The successful extraction of genomic DNA from left and right ganglia was confirmed by PCR utilizing primers designed for the murine housekeeping gene G3PDH (385 bp).

FIG. 8 shows injection of FIV(lacZ) in the right TMJ (5×10⁶ infectious particles) resulted in successful transduction of primary sensory neurons with the reporter gene β-galactosidase in trigeminal ganglia ispilateral to the treated joint. (A) β-galactosidase expression was detected by X-gal histochemistry in sagittal sections of right-side ganglion (4×), (B) primarily at its posterior and posterolateral region (20×). (C) Injection of FIV(Δ'lac) did not result in β-galactosidase expression. (D) The X-gal staining was confirmed with immunocytochemistry employing antibodies raised against bacterial β-galactosidase following FIV(lacZ) injection compared to (E) FIV(Δ'lac) treatment.

FIG. 9 shows The neuronal cell line N2α was infected with HIV(HUMOR) [a Lenti virus]. Total RNA was extracted from infected and control cells. The levels of HUMOR and G3PDH transcript were assessed by RT-PCR. Minimal amounts of HUMOR were detected in naïve cells (C1, C2), as well as cells infected with the HIV(lacZ) virus (L1, L2). In contrast, HUMOR was readily detected in cells infected with HIV(HUMOR). As a control, the housekeeping gene G3PDH transcript was detected in all samples analyzed.

### IV. DETAILED DESCRIPTION

Before the present compounds, compositions, articles, devices, and/or methods are disclosed and described, it is to be understood that the disclosure is not limited to specific synthetic methods or specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

Disclosed are the components to be used to prepare the disclosed compositions as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a particular μ-opioid receptor vector is disclosed and discussed and a number of modifications that can be made to a number of molecules including the μ-opioid receptor vector are discussed, specifically contemplated is each and every combination and permutation of the μ-opioid receptor vector and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods.

### A. DEFINITIONS

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” as well as “less than” and “greater than” 10 are also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15.

In this specification and in the claims which follow, reference will be made to a number of terms which shall be defined to have the following meanings:

“Optional” or “optionally” means that the subsequently described event or circumstance may or may not occur, and that the description includes instances where said event or circumstance occurs and instances where it does not.

“Primers” are a subset of probes which are capable of supporting some type of enzymatic manipulation and which can hybridize with a target nucleic acid such that the enzymatic manipulation can occur. A primer can be made from any combination of nucleotides or nucleotide derivatives or analogs available in the art which do not interfere with the enzymatic manipulation.

“Probes” are molecules capable of interacting with a target nucleic acid, typically in a sequence specific manner, for example through hybridization. The hybridization of nucleic acids is well understood in the art and discussed herein. Typically a probe can be made from any combination of nucleotides or nucleotide derivatives or analogs available in the art.

Throughout this application, various publications are referenced. The disclosures of these publications in their entireties are hereby incorporated by reference into this application in order to more fully describe the state of the art to which this invention pertains. The references disclosed are also individually and specifically incorporated by reference herein at least for the material contained in them that is discussed in the sentence in which the reference is relied upon.

### B. COMPOSITIONS AND METHODS

The μ-opioid receptor (HUMOR) is a key component of the intrinsic anti-nociceptive pathway in mammals: descending bulbospinal serotonergic and noradrenergic neuronal projections exert anti-nociceptive effects via release of endogenous opioids, which in turn activate μ-opioid receptors present on the presynaptic membrane of the primary sensory neurons. Pain stimulus travels through the nerve to the brain through activation of nociceptors. The activation of μ-opioid receptors through binding of opioids interrupts the transmission of the pain signal. Mammals release endogenous opioids when under pain assault and billions are spent each year in the pharmaceutical industry to treat pain through the administration of opioids and opioid like molecules that target the μ-opioid receptors. Disclosed herein are compositions and methods for the treatment of pain, which do not require the administration of μ-opioid receptor targeted molecules or utilize lower effective amounts of opioid receptor targeted molecules. The disclosed methods involve the over-expression of μ-opioid receptors, which can make the nerve cell more receptive to endogenous opioid molecules or to opioids or opioid like molecules administered as a pharmaceutical. Over expression of the μ-opioid receptors can occur through simulation of endogenous opioid receptor genes or through transgenic therapy that delivers a construct encoding the opioid receptor.

Receptor up-regulation is designed to result in circumventing the observed desensitization following prolonged opioid drug administration, which in part occurs as a decrease in receptor expression. Furthermore, the strategy can take advantage of the existing intrinsic anti-nociceptive mechanism by ensuring adequate μ-opioid receptor presence at the site of the central processing of pain. This adequate receptor presence is consistent with heightened sensitivity of patients to drugs administered exogenously, which is consistent with requiring smaller doses of opioid analgesics, such as Ultram, Fentanil, and Darvon, which otherwise commonly result to pathologic addiction.

Disclosed are compositions and methods to target the expression of opioid receptors, such as the μ-opioid receptors, such as human opioid receptors, to sensory neurons innervating regions that can experience pain, such as orofacial regions that experience nociception. Disclosed are compositions and methods for targeting opioid receptors, such as the μ-opioid receptors, expression in sensory orofacial neurons. Also disclosed are compositions and methods for targeting opioid receptors, such as the μ-opioid receptors, expression in any sensory neuron. For example, the compositions and methods can be used in any sensory neuron, wherein the sensory neuron processes pain or other “input” signals from peripheral tissues (e.g., joints, amputated limbs, extracted or endodontically treated teeth), as well as vital organs. For example, disclosed are vectors, such as feline immunodeficiency lentiviral vectors (FIV), rAAV vectors, HSV Amplicon, and liposomes for delivery of the opioid receptor DNA. Administration of the vectors peripherally to infect those sensory neurons, such as those innervating the orofacial region, can be performed For example, the vectors can be delivered at the point of pain, for example, an extremity, by for example, injection into the extremity. Disclosed are vectors, such as FIV, rAAV, HSV Amplicon, and liposomes, capable of stably transducing terminally differentiated cells, including neurons.

a) Nervous System

The nervous system can be divided into two parts: central and peripheral. The central nervous system consists of the encephalon or brain and the medulla spinalis or spinal cord. These two parts, the brain and the spinal cord are continuous with on another at the level of the upper border of the atlas vertebra. The peripheral nervous system consists of a series of nerves, which connect the central nervous system to all of the tissues in the body. Nerves also are often grouped as cerebrospinal and sympathetic. However, since the two groups are intimately connected and closely intermingled these distinctions are not absolute. Nerve cells can also be classified as efferent or afferent nerves. Efferent nerve cells are nerve cells that transmit signals from the brain to the periphery and afferent nerve cells are nerve cells that transmit signals from the periphery to the brain.

Neurons act as pain pathways and these pathways include peripheral, spinal, and supraspinal elements. The peripheral part of the system includes the primary afferent sensory neurons. These neurons are called nociceptors, and can be found throughout the body, such as in the skin, muscle, connective tissue, the cardiac system, and abdominal and thoracic viscera. Nociceptors are uncapsulated nerve endings that detect thermal, mechanical, or chemical stimuli, and are thus, not small molecule receptors. Nociceptors can be thinly myelinated or unmyelinated nerve fibers. The thinly myelinated variety are termed A-delta fibers and the unmyelinated variety are termed C-polymodal fibers. The primary functional difference between A and C delta fibers is that A-delta fibers are rapidly conducting and C delta fibers are slowly conducting. This means that A delta fibers transmit sensations perceived as fast, sharp, well-localized pricking pain, and C-polymodal fibers transmit feeling via thermal, mechanical, and chemical stimuli transmitting sensations perceived as dull, aching, burning, poorly localized pain.

Most A-delta and the C-polymodal afferent fibers enter the dorsal horn of the spinal cord by way of the dorsal nerve roots and their ganglia. Wide dynamic range neurons receive nociceptive and non-nociceptive input from the skin, muscle, and viscera. This convergence can account for visceral referred pain. Impulses are then transmitted to the brain by the spinal thalamic tract (STT). Near the thalamus, the SIT bifurcates into the neospinothalamic tract and the paleospinothalamic tract, projecting to the thalamus, hypothalamus, periaqueductal gray matter (PAG) in the brain stem. The thalamus processes sensory input is projected to the cerebral cortex, basal ganglia, and limbic system. Descending pathways conduct transmission from the brain to the spinal cord control and modify afferent sensory input.

Nociception can be thought of as the detection of tissue damage by nociceptors. Modulation of nociception occurs peripherally, spinally, and supraspinally. Tissue damage is associated with the release of chemical mediators, such as serotonin, histamine, bradykinin, cytokines, prostaglandins, and leukotrienes, which produce inflammation, and occurs in the peripheral system. The pain transmission is modulated by these events and this lowers excitability threshold of the nociceptor threshold so that stimuli normally non-painful stimuli become painful. This is called nociceptor sensitization. Two other substances that sensitize nociceptors are substance P and glutamate, which can be released from nerve terminals.

The signals from the nociceptors are processed in the dorsal horn of the spine. Repetitive, convergent input from A-delta and C polymodal fibers at the dorsal horn can result in a state where less stimulation is required for the generation of a pain response. This is known as the wind-up phenomenon, and is thought to be initiated by the release of substance P and the excitatory amino acids glutamate and aspartate.

The brain also signals the spinal cord to modulate the pain response. The PAG region of the brainstem contains high concentrations of opioid receptors, and sends projections to the rostral medulla and eventually to the dorsal root inhibiting ascending pain impulses. Thus, the activation of the opioid receptors interrupts the transmission of the pain signal. Descending pathways can also stimulate spinal nociceptive transmission as well.

b) Pain

Pain is typically classified into two categories: nociceptive pain (somatic pain) and neuropathic pain. Nociceptive pain is pain that is sensed after some type of trauma. The nociceptive pain is sensed by the “nociceptor” sensory fibers which are connected to the nervous system. After an injury to a muscle, soft tissue (ligaments, tendons), bones, joints, or skin (or other organs), these sensory fibers are stimulated which causes a transmission of a signal through an afferent neuron to the brain. Nociceptive pain is often characterized as a deep aching, throbbing, gnawing, or sore sensation. Common examples of nociceptive pain include: pain after trauma (e.g. a car accident or a fall), postoperative pain, and arthritis pain. Nociceptive pain is usually localized and gets better with healing.

Neuropathic pain is pain caused by damage to nerve tissue. Neuropathic pain is often characterized as burning, severe shooting pains, and/or persistent numbness or tingling. Common examples of neuropathic pain related to back pain include sciatica, pain that travels from the spine down the arm, and pain that persists after back surgery.

It is thought that in some cases prolonged nociceptive pain may progress to neuropathic pain, and a patient may have both nociceptive and neuropathic pain at the same time. Pain is also often classified as acute pain or chronic pain. Acute pain is characterized as pain where the amount of pain directly correlates with the level and duration of tissue damage. Acute pain therefore, provides a protective reflex, such as the reflex to move your hand immediately if you touch a sharp object. This type of pain is a symptom of injured or diseased tissue, so that when the underlying problem is cured the pain goes away. Acute pain is a form of nociceptive pain. Chronic pain on the other hand, does not correlate with the severity of the insult, and therefore, typically will not serve a protective function. Prolonged damage to tissues, i.e. knee pain or tooth ache, will eventually result in plastic (non reversible) changes in the neurons that process pain from that area, which now facilitate either allodynia and/or hyperalgesia. Chronic pain is born following these plastic neuronal changes, whereby the neurons are now “sick” and pain will occur even in the absence of peripheral stimulus (e.g., amputated limbs, extracted teeth). In fact, its basis is neuropathic now, and neurons continuously send pain messages to the brain even though there is no continuing tissue damage. Neuropathic pain is a form of chronic pain.

(1) Anatomy of Orofacial Pain

The mandibular division of the trigeminal nerve provides sensory innervation to the TMJ and masticatory muscles. The cell bodies of these primary sensory neurons are located in the inferior portion of the trigeminal ganglion extending their unmyelinated (C-fibers) or thinly myelinated (Aδ-fibers) peripheral projections to structures of the face and jaws. More specifically, nociceptive innervation to the temporomandibular joint (TMJ) is primarily provided by the auriculotemporal nerve of the mandibular division of the trigeminal nerve (Sessle B J, Hu J W (1991). Can J Physiol Pharmacol 69: 617-626). Aδ and C nerve fibers, whose cell bodies are located in the posterolateral part of the trigeminal ganglion (Yoshino K, et al. (1998). Arc Oral Biol; 43: 679-686), project distally and terminate as non-encapsulated free nerve endings dispersed throughout the posterolateral part of the TMJ capsule (Bernick S (1962). Oral Surg 15:488-492; Thilander B (1964). Acta Odont Scan 22:151-156; Frommer J, Monroe C W (1966). J Dent Res 45:1762-1766; Klineberg I (1971). Ann Royal Coll Surg Engl 49:268-288), the posterior band of the meniscus and the posterior attachment (Dressen D, et al. (1990). Acta Anat 139:154-160; Kido M A, et al. (1991). Arch Oral Biol 36:397-400, Kido M A, et al. (1993). J Dent Res 72:592-598; Wink C S, et al. (1992). J Oral Maxillofac Surg 50:334-337). Inflammation, injury or other agents may cause excitation of the free and unspecialized nerve endings of the unmyelinated C-fibers, which are predominately involved in the transmission of nociception from the TMJ, muscles of mastication as well as the pulp of teeth. The central projections enter the brain stem via the ventrolateral pons, descend caudally as the dorsolateral trigeminal tract and synapse with second order sensory neurons at the substantia gelatinosa of the subnucleus caudalis of the descending trigeminal nucleus (medullary dorsal horn). Second order sensory neurons extend projections to the nucleus proprius, followed by subsequent projections to the intermedial gray, and then to the reticular formation of the brain stem, and through the intralaminal nuclei of the thalamus project wide spread connections into the cortex. The ascending sensory neural architecture is also susceptible to an intrinsic opioid-releasing anti-nociceptive descending system, the inhibitory effects of which are mediated by opioid receptors expressed in the presynaptic membrane of the primary sensory neurons. Although pain is initially elicited at a peripheral site, it is further centrally modulated, i.e. in the brain, enhanced or attenuated, therefore making this aforementioned central processing of pain a major component in sensory orofacial nociception.

In the quest for developing new therapies for orofacial pain, gene therapy appears to be an emerging treatment method (Kuboki T, et al. (1999). Arc Oral Biol 44: 701-709; Pohl M, Braz J (2001). Eur J Pharmacol 429: 39-48; Baum B J, et al. (2002). JADA 133: 35-44). For example, it has been previously suggested that delivery of antisense oligonucleotides developed against nociceptive genes to appropriate tissues may offer alternatives in designing novel treatments for pain management (Wu C L, et al. (2001). Anesthesiology 95: 216-240).

Disclosed herein, transfer of anti-nociceptive genes to sensory trigeminal neurons innervating the orofacial region can be achieved after injection of lentiviral vectors at the painful site, such as the TMJ, resulting in their uptake by free nerve endings and retrograde transport to the sensory cells' nuclei. Previous studies demonstrated axonal retrograde transport of horseradish peroxidase from the TMJ to the central nervous system (Romfh J H, et al. (1979). Exp Neurol 65: 99-106; Capra N F (1987). Somatosensory Res 4: 201-213) including the trigeminal ganglia (Yoshino K, et al. (1998). Arc Oral Biol; 43: 679-686). In evaluating the employment of lentiviral vectors as the basis for TMJ gene therapy, as an example, VSV-G pseudotyped feline immunodeficiency viral vectors (FIV) were used. These vectors are capable of stably transducing dividing, growth arrested as well as post-mitotic cells, as it is capable of transgene integration into the host's genome (Poeschla E M, et al. (1998). Nature Med 4: 354-357). VSV-G pseudotyping of viral vectors confers a broad range of host specificity, including human and murine cells, as infection is mediated by the interaction of the viral envelope protein and a phospholipid component of the cell membrane leading to membrane-fusion mediated entry (Burns J C, et al. (1993). Proc Natl Acad Sci USA 90: 8033-8037; Carneiro F A, et al. (2002). J Virol 76: 3756-3764). Therefore, FIV vectors can mediate sustained gene expression in non-dividing terminally differentiated trigeminal sensory neurons, a property unique to lentiviral vectors.

c) Current Pharmacologic Agents in the Management of Pain

Non-steroidal anti-inflammatory drugs (NSAID's) are often utilized as the first line of agents for the management of pain. NSAID's primarily exert their pain-killing effects by inhibiting the production of prostanoids and attenuating peripheral inflammatory conditions that may be responsible for pain elicitation. Alternatively, corticosteroids may be utilized with peripheral routes of action. In contrast, exogenously administered opioid drugs (morphine) mimic the effects of the endogenous opioids by crossing the blood brain barrier (BBB). Similarly, tricyclic antidepressants that cross the BBB have been also employed in cases of chronic pain by inhibiting the reuptake of serotonin and norepinephrine. However, each of these four classes of drugs is characterized by significant side effects that prohibit their long term use as well as often show unfavorable treatment outcomes.

d) Opioid Receptors and Mechanism of Action

Opioid analgesics have been used for pain management for hundreds of years. Opium itself consists of the dried latex from the unripe fruit of the opium poppy Papaver somniferum. Morphine is isolated from opium. Opioid receptors exist in the spinal and supraspinal regions of the nervous systems. Opioids can modulate neuronal transmission by binding to opioid receptors in the dorsal-root ganglia, the central terminals of primary afferent neurons (LaMotte C, et al., Brain Res 1976; 112:407-12; Fields H L, et al., Nature 1980; 284:351-3) and peripheral sensory-nerve fibers and their terminals (Stein C, et al., Proc Natl Acad Sci USA 1990; 87:5935-9; Hassan A H S, et al., Neuroscience 1993; 55:185-95. The dorsal-root ganglia and trigeminal ganglion (Xie G X, et al., Life Sciences 1999; 64:2029-37; Li J L, et al., Brain Res 1998; 794:347-52.) contain messenger RNA (mRNA) for opioid receptors (Schafer M, et al., Eur J Pharmacol 1995; 279:165-9; Mansour A, et al., Brain Res 1994; 643:245-65) and primary afferent nerves mediate the peripheral antinociceptive effects of morphine (Bartho L, et al., Naunyn Schmiedebergs Arch Pharmacol 1990; 342:666-70). The presence of endogenous opioids and their receptors are able to produce a potent anti-nociception. Opioids increase potassium currents and decrease calcium currents in the cell bodies of sensory neurons (Werz M A, Macdonald R L., Neurosci Lett 1983; 42:173-8; Schroeder J E, et al., Neuron 1991; 6:13-20), both of which can lead to the inhibition of neuronal firing and transmitter release. A similar process occurring throughout the neuron, can explain why opioids attenuate both the excitability of the peripheral nociceptive terminals and the propagation of action potentials (Andreev N, et al., Neuroscience 1994; 58:793-8; Russell N J W, et al., Neurosci Lett 1987; 76:107-12). At central nerve terminals, (Yaksh T L, et al., Nature 1980; 286:155-7) opioids inhibit the calcium-dependent release of excitatory, pro-inflammatory compounds (e.g., substance P) from peripheral sensory-nerve endings, (Yaksh T L., Brain Res 1988 458:319-24) which contribute to the anti-inflammatory actions of opioids Barber A, Gottschlich R. et al., Med Res Rev 1992; 12:525-62).

There are three known opioid receptors, μ, κ, and δ-opioid receptors. μ-Receptors are further subdivided into at least two subclasses, μ1 and μ2-receptors. The body produces opioid like molecules, called endogenous opioids, such as endorphins, enkephalins, and dynorphins. μ-receptors are known to mediate analgesia, respiratory depression, bradycardia, nausea/vomiting, and decreased gastrointestinal tone.

δ-receptors mediate supraspinal analgesia, and κ-receptors mediate dysphoria and tachycardia. As pain impulses ascend through the spinal and supraspinal nervous system, activation of the opioid receptors inhibits these impulses and inhibits the transmission of pain from the dorsal horn. In addition, opioid analgesics prevent the presynaptic release of pain mediators such as Substance P into the spinal cord region.

All animals, such as mammals, such as human, contain opioid receptors. It is understood that there are homologs for the various opioid receptors across animal species. A number of different opioid receptor sequences are set forth in the SEQ IDS, including μ-opioid receptors. The human μ-opioid receptor is referred to herein as HUMOR. It is understood that if a particular statement or reference is made regarding HUMOR that this statement is equally applicable to homologous receptors, unless specifically indicated otherwise.

Opioid analgesics are typically grouped into three classes: naturally occurring (morphine, codeine); semi-synthetic morphine derivatives (hydromorphone, oxycodone, hydrocodone); and synthetic. Synthetic opioid analgesics include morphinan derivatives (levorphanol); methadone derivatives (methadone, propoxyphene); benzomorphan derivatives pentazocine); and phenylpiperidine derivatives (meperidine, fentanyl, sufentanil, alfentanil, remifentanil). Tramadol is an opioid analgesic that also inhibits the reabsorption of norepinephrine and serotonin.

Another way to classify opioid analgesics is as agonists, partial agonists, mixed agonists/antagonists, and antagonists based on their interactions at the opioid receptors. μ, and κ opioid-receptors are stimulated by agonists. Partial agonists have reduced μ-opioid receptor activity, and mixed agonists/antagonists only stimulate certain μ and κ-opioid receptors. Antagonists bind μ and κ-opioid receptors but do not stimulate the receptor activity.

Some agonists are Morphine, Hydromorphone, Oxymorphine, Codeine, Oxycodone, Hydrocodone, Dihydrocodeine, Methadone, Meperidine, Fentanyl, Sufentanil, Alfentanil, and Remifentanil. An example of a partial agonist is Buprenorphine. Pentazocine, Nalbuphine, and Butorphanol are examples of mixed agonists/antagonists. Examples of antagonists are Naloxone and Nalmefene. It is understood that one way to classify opioid receptors is by which molecules act as antagonists and which act as agonists, for example. Thus, a receptor can be defined as “a receptor for which morphine is an agonist.”

There are a number of adverse side effects that can occur when taking opioid analgesics, such as CNS effects, such as sedation, confusion, and respiratory depression. Gastrointestinal adverse effects include nausea, vomiting, and constipation. Involuntary muscular contractions (twitching) known as myoclonus, bradycardia, and hypotension, can also occur. Lastly, physical and psychological dependence can also occur upon use or prolonged use of opioid analgesics. Thus there is a significant need for the disclosed compositions and methods, which reduce or eliminate the need for opioid analgesics in many indications.

e) β-Opioid Receptor Therapy

The disclosed approach for the management of pain involves the targeted expression of opioid receptor(s) such as the μ-opioid receptor in the primary neurons innervating the areas, such as orofacial areas, experiencing pain, resulting in these same neurons becoming more susceptible to the intrinsic opioid anti-nociceptive mechanisms. Disclosed are compositions and methods for treating pain. The compositions comprise an opioid receptor that is expressed from a vector. Typically these compositions will be delivered to at the point of pain. It is thought that their expression, increases the efficiency of the body's own opioid like molecules and decreases pain.

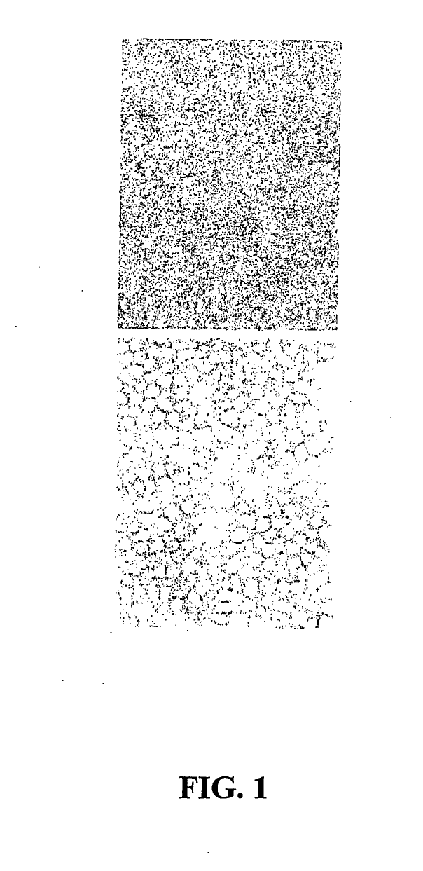

Disclosed herein, the cDNA for a human α-opioid receptor (HUMOR) is set forth in SEQ ID NO:2. The μ-opioid receptor (Raynor K, et al., J Pharmacol Exp Ther. 1995; 272:423-8) has been placed into a vector herein and expressed in primary fibroblasts as well as cells of the N2a neuronal cell line (FIG. 1). Transduction and stable expression of μ-opioid receptor in neurons can be accomplished by employing VSV-G pseudotyped immunodeficiency viral vectors (FIV).

The expression of the μ-opioid receptor in the neurons at the point of pain in certain embodiments requires transduction in a non-dividing cell such as a neuron. This can be accomplished using a transduction mechanism, such as lipofection or encapsulation methods, or via viral vector systems that function with cell division, such a lentiviruses, such as the FIV virus, or adeno-associated viruses, rAAV vectors, HSV Amplicon, and liposomes.

It has been previously shown that this FIV system is capable, due to its lentiviral properties, of infecting terminally differentiated cells, including neurons. Disclosed are methods for administering vectors, such as the FIV(μ-opioid receptor) vector, peripherally at the site of pain. The neurons innervating that specific site and mediating the nociceptive signals are infected and stably transduced. These vectors, including vectors expressing lacZ and the μ-opioid receptor, can transduce nerve cells in vivo, in mice, through injection at the periphery.

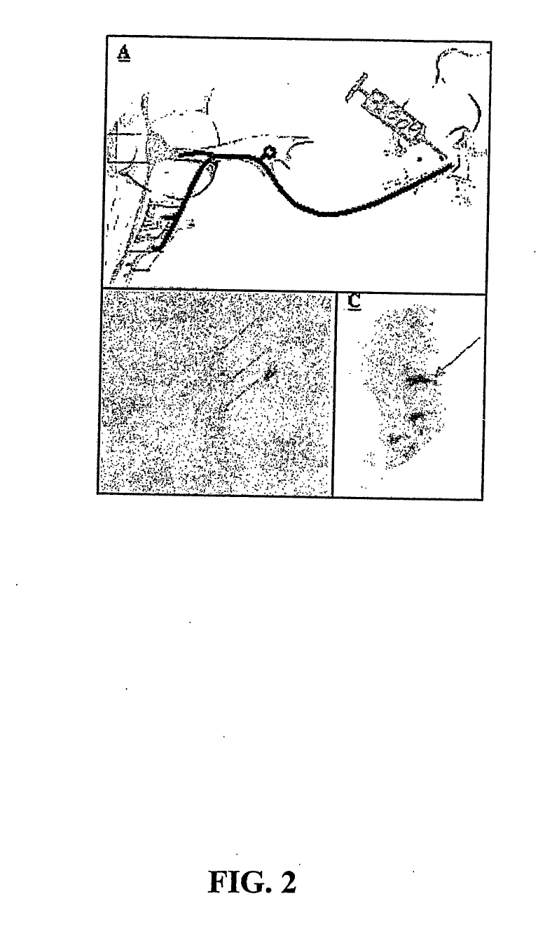

Disclosed herein is the stable expression of a reporter gene, the lacZ gene, in neurons located in the appropriate region of the trigeminal ganglion following peripheral injection of FIV(lacZ) in the area of the TMJ (FIG. 2), as well as a variety of expression vectors containing the μ-opioid receptor, such as the human μ-opioid receptor.

Disclosed are vectors wherein the vector includes sequence encoding the 1-opioid receptor gene. Also disclosed are vectors, wherein a μ-opioid receptor gene has been cloned in an FIV vector. Disclosed are methods comprising administering the disclosed vectors to cells, including cells involved in transmitting pain signals, such as nerve cells in the orofacial regions, related to for example, pain from TMJ and the masseter muscle.

Also disclosed are transgenic mice that have been stably transfected with the disclosed vectors. These mice can be used, for example, as models of pain and the testing of therapeutics.

f) Mouse Model of Experimental Nociception

The majority of models evaluate reflex increases in jaw muscle activity, activating putative nociceptive pathways by the injection of algesic substances. Broton and Sessle (Boton J G, Sessle B J, et al., Arch Oral Biol 1988; 33:741-47.) placed hypertonic saline, potassium chloride and histamine into the TMJ of cats and found increase in electromyographic (EMG) activity in the temporal, digastric and genioglossus muscles. This suggests that the products of tissue injury and or inflammation are capable of producing pain within the TMJ, causing excitation of sensory afferents and reflex muscle activity. Tambeli et al. (Tambeli C H, et al., J Dent Res 1997; 76: (Special Issue) abstr# 1263.) injected mustard oil unilaterally into the TMJ and the results were similar to Broton and Sessle (Boton J G, Sessle B J, et al., Arch Oral Biol 1988; 33:741-47.). Increases in masticatory muscle activity have been also demonstrated by injection of excitatory amino acids into the TMJ area. Cairns et al. (Cairns B E, et al., J Neurosci 18; 1998:8056-64. Cairns B E, et al., J Neurophysiol 1999; 81: 1966-69.) injected glutamate into the TMJ area of rats and in a dose dependent manner induced a prolonged increase in EMG activity by the excitation of nociceptors (A delta and C fibers) (Cairns B E, et al., J Neurophysiol 2001; 86: 782-90). Cairns et al. (Cairns B E, et al., J Neurophysiol 85; 2001:2446-54.) placed glutamate into the masseter muscle of the rat and human and evoked higher masseter muscle activity in female than males of both species. These results indicate that peripheral insult can produce pain behavior and changes in resting muscle activity when algogenic substances are injected into the TMJ and masticatory muscles. Disclosed are behavioral models to assess orofacial pain from the TMJ and masseter muscle via application of glutamate into the TMJ and masticatory muscles. These models can include mice, disclosed herein, which have had vectors encoding μ-opioid receptor stably transduced. Resistance to jaw opening and EMG activity can serve as behavioral measures of pain.

### C. COMPOSITIONS

1. Opioid Receptors

There are typically considered three classes of opioid receptor μ, δ and κ. Genes encoding for these receptors have been cloned (Evans et al (1992) Science 258 1952; Kieffer et al (1992) Proc. Natl. Acad. Sci. USA 89 12048; Chen et al (1993) Mol. Pharmacol. 44 8; and Minami et al (1993) FEBS Lett. 329 291 all of which are herein incorporated by reference for material related to opioid receptors and there sequence). In addition, an orphan receptor was identified which has a high degree of homology to the known opioid receptors and based on structural grounds it is considered a receptor called ORL1 (opioid receptor-like) (Mollereau et al (1994) FEBS Lett. 341 33, herein incorporated by reference for material related to opioid receptors and there sequence). Since the cloned receptors function as opioid receptors, by for example interacting with pertussis toxin-sensitive G-proteins, all of the cloned opioid receptors possess the same general structure which includes an extracellular N-terminal region, seven transmembrane domains and intracellular C-terminal tail structure. Evidence obtained from pharmacokinetic and activity data indicate there are subtypes of each receptor and other types, such as less well-characterized opioid receptors, such as ε, λ, τ, ζ, which are known. One way of characterizing the different receptor subtypes for μ-, δ- and κ-receptors is through different post-translational modifications of the gene product (glycosylation, palmitoylation, phosphorylation, etc). Also receptor dimerization to form homomeric and heteromeric complexes or from interaction of the gene product with associated proteins such as RAMPs can effect function, and thus represent another way to characterize the receptors. Different opioids have different affinity for the different opioid receptors. For example, μ-morphine, δ-leukenkephalin metenkephalin, κ-dynorphin, β-endorphin, have different affinities for the various opioid receptors.

a) μ-Receptor Subtypes

The MOR-1 gene, encoding for one form of the μ-receptor, shows approximately 50-70% homology to the genes encoding for the δ-(DOR-1), κ-(KOR-1) and orphan (ORL1) receptors. Two different splice variants of the MOR-1 gene have been cloned, and they differ by 8 amino acids in the C-terminal tail which are either present or not. The splice variants exhibit differences in their rate of onset and recovery from agonist-induced internalization but their pharmacology does not appear to differ in ligand binding assays. A MOR-1 knockout mouse has been made and the mouse does not respond to morphine, by failing to alleviate pain, and by failing to exhibit positive reinforcing properties or an ability to induce physical dependence in the absence of the MOR-1 gene. This indicates that at least in this species, morphine's analgesia is not mediated through δ- or κ-receptors. (Matthes et al (1996) Nature 383 818).

The μ receptor is divided into the μ1 and μ2 groups. The division occurs because of binding and pharmaco activity studies which indicate, for example, that naloxazone and naloxonazine abolish the binding of radioligands to the μ1-site, and in vivo studies showed that naloxazone selectively blocked morphine-induced antinociception but did not block morphine-induced respiratory depression or the induction of morphine dependence, indicating different types of μ-receptor (Ling et al (1984) Science 226 462 and Ling et al (1985) J. Pharmacol. Exp. Ther. 232 149). Subsequent work in other laboratories has failed to confirm this classification.

Peptide sequences of the human and mouse μ receptor are set forth in SEQ ID Nos 1 and 3 respectively.

There is also data consistent with a third form of μ receptor where analogues of morphine with substitutions at the 6 position (e.g. morphine-6b-glucuronide, heroin and 6-acetyl morphine) are agonists, but with which morphine itself does not interact (Rossi et al (1996) Neuroscience Letters 216 1, herein incorporated by reference for material at least related to opioid receptors and their function and structure). Antinociception tests on mice show that morphine does not exhibit cross tolerance with morphine-6b-glucuronide, heroin or 6-acetyl morphine. Furthermore, in mice of the CXBX strain morphine is a poor antinociceptive agent whereas morphine-6b-glucuronide, heroin and 6-acetyl morphine are all potently antinociceptive. Heroin and morphine-6-glucuronide, but not morphine, still produce antinociception in MOR-1 knockout mice in which the disruption in the MOR-1 gene was engineered in exon-1 (Schuller et al (1999) Nature Neuroscience 2 151). Furthermore, all three agonists were ineffective as antinociceptive agents, in MOR-1 knockout mice in which exon-2, not exon-1, had been disrupted. This indicates that the antinociceptive actions of heroin and morphine-6-glucuronide in the exon-1 MOR-1 mutant mice are mediated through a receptor produced from an alternative transcript of the MOR-1 gene differing from the MOR-1 gene product, the μ-opioid receptor, in the exon-1 region.

b) δ-Receptor Subtypes

Only one δ-receptor gene has been cloned (DOR-1), but overlapping subdivisions of δ-receptor have been proposed (δ1/δ2 and δcx/δncx) on the basis of in vivo and in vitro pharmacological experiments.

The δ receptor subclasses arise from pharmacological studies. Results from in vivo rodent studies are shown in Table 1.

TABLE 1

| | | | Non-competitive |
|---|---|---|---|
| | Agonist | Competitive antagonist | antagonist |
| δ1 | DPDPE/DADLE | BNTX (7- | DALCE ([D- |
| | | benzylidenenaltrexone) | Ala2, D- |
| | | | Leu5]enkephalyl- |
| | | | Cys) |
| δ2 | Deltorphin II/ | Naltriben | 5′-NTII |
| | DSLET | | (naltrindole 5′- |
| | | | isothiocyanate |

There are a number of different ligands for the opioid receptors which differentially bind one or more receptors. Examples of these ligands are shown in Table 2.

TABLE 2

| | Receptor type | | | |
|---|---|---|---|---|
| | μ-Receptor | δ-Receptor | κ-Receptor | ORL₁ |
| Selective | endomorphin- | [D-Ala²]- | enadoline | nociceptin/OFQAc- |
| agonists | 1endomorphin- | deltorphin I | U-50488 | RYYRWK-NH₂* |
| | 2DAMGO | [D-Ala²]- | U-69593 | |
| | | deltorphin II | | |
| | | DPDPE SNC 80 | | |
| Selective | yICI 174864 | binaltorphimine | Selective | None as yet** |
| antagonists | nor | | antagonists | |
| CTAP | | | CTAP | |
| naltrindole | | | naltrindole | |
| TIPP | | | TIPP | |
| Radioligands | [3H] | DAMGO [3H] | [³H]-enadoline | [³H]-nociceptin |
| | | | [³H]-U69593 | |

The pharmacological properties of the cloned DOR-1 receptor are somewhere between those predicted for either the δ1 or δ2 subtypes. Mouse and human recombinant receptors both bind DPDPE and deltorphin II, which can displacer of [3H]-diprenorphine. This is different than either a δ1 or δ2 classification (Law et al (1994) J. Pharmacol. Exp. Ther. 271 1686). [3H]-diprenorphine binding to the mouse recombinant receptor, however, is more highly displaced by naltriben than BNTX, consistent with it being δ2 like.

Opioid receptors have also been indicated to be in complex μ-receptors and κ-receptors. For example, one type of δ receptor subtypes complexes, δcx, and another appears not to complex, δncx (Rothman et al (1993) In: Handbook Exp. Pharmacol. Ed. A. Herz 104/1 p 217).

c) κ-Receptor

The cloned κ-Receptor has the sequence set forth in SEQ ID NO: 5, which represents an example of a κ-receptor.

d) The Orphan Opioid Receptor

The orphan receptor has been identified in three species: rat, mouse and man, all having a greater than 90% identity with each other. This receptor is typically referred to as ORL-1 for orphan receptor like 1. The endogenous peptide agonist for ORL1 is known as nociceptin or orphanin FQ. While the ORL1 receptor has structural homology to orphan receptors it does not have pharmacological homology. Non-selective ligands that exhibit high affinity for all μ-, κ- and δ-receptors, have very low affinity for the ORL1 receptor. Comparison of the deduced amino-acid sequences of the four receptors highlights structural differences consistent with the lack of coligand binding. The trans-membrane regions are conserved near their top in the μ-, κ- and δ-receptors, but are altered in ORL1. Site-directed mutants of ORL1 towards the traditional receptors (rat) are able to bind the traditional receptor's ligands. For example, benzomorphan bremazocine binds ORL1 by changing Ala213 in TM5 to the conserved Lys of μ, κ and δ, or by changing the Val-Gln-Val276-278 sequence of TM6 to the conserved Ile-His-Ile motif (Meng et al (1996) J. Biol. Chem. 271 32016). There are also a number of splice variants of the ORL1 receptor, XOR (Wang et al (1994) FEBS Lett. 348 75) with a long form (XOR1L) containing an additional 28 amino acids in the third extracellular loop and in the homologous receptor from mouse, KOR-3, five splice variants have been reported to date (Pan et al (1998) FEBS Lett. 435 65).

e) Endogenous Ligands

In mammals the endogenous opioid peptides are mainly derived from four precursors: pro-opiomelanocortin, pro-enkephalin, pro-dynorphin and pro-nociceptin/orphanin FQ. Nociceptin/orphanin FQ is processed from pro-nociceptin/orphanin FQ and is the endogenous ligand for the ORL1-receptor; it has little affinity for the μ, δ- and κ-receptors. Table 3 sets forth endogenous ligands for the opioid receptors. These peptides bind μ, δ- and κ-receptors with different affinity, and have negligible affinity for ORL1-receptors, but none binds exclusively to one opioid receptor type. β-endorphin is equiactive at μ-and δ-receptors with much lower affinity for κ-receptors; the post-translational product, N-acetyl-β-endorphin, has very low affinity for any of the opioid receptors. [Met]- and [Leu]enkephalin have high affinities for δ-receptors, ten-fold lower affinities for μ-receptors and negligible affinity for κ-receptors. Other products of processing of pro-enkephalin, which are N-terminal extensions of [Met]enkephalin, have a decreased preference for the δ-receptor with some products, e.g. metorphamide displaying highest affinity for the μ-receptor. The opioid fragments of pro-dynorphin, particularly dynorphin A and dynorphin B, have high affinity for κ-receptors but also have significant affinity for μ- and δ-receptors.

Endomorphin-1 and endomorphin-2 are putative products of an as yet unidentified precursor, that have been proposed to be the endogenous ligands for the μ-receptor where they are highly selective. The endomorphins are amidated tetrapeptides and are structurally unrelated to the other endogenous opioid peptides (Table 3). Although the study of the cellular localization of these peptides is at an early stage, endomorphin-2 is found in discrete regions of rat brain, some of which are known to contain high concentrations of μ-receptors. Endomorphin-2 is also present in primary sensory neurones and the dorsal horn of the spinal cord where it could function to modulate nociceptive input.

In comparison to the mainly non-selective mammalian opioid peptides (the exceptions being the endomorphins), amphibian skin contains two families of D-amino acid-containing peptides that are selective for μ- or δ-receptors. Dermorphin is a μ-selective heptapeptide Tyr-D-Ala-Phe-Gly-Tyr-Pro-Ser-NH2 without significant affinity at d- and k-receptors. In contrast, the deltorphins-deltorphin (dermenkephalin; Tyr-D-Met-Phe-His-Leu-Met-Asp-NH2), [D-Ala2]-deltorphin I and [D-Ala2]-deltorphin II (Tyr-D-Ala-Phe-Xaa-Val-Val-Gly-NH2, where Xaa is Asp or Glu respectively)—are highly selective for δ-opioid receptors. Table 3 shows a variety of endogenous opioid receptor molecules.

TABLE 3

| Precursor | Endogenous peptide | Amino acid sequence |
|---|---|---|
| Pro- | β-Endorphin | YGGFMTSEKSQTPLVTL- |
| opiomelanocortin | | FKNAIIKNAYKKGE |
| Pro-enkephalin | [Met]enkephalin | YGGFM |
| | [Leu]enkephalin | YGGFL |
| | | YGGFMRF |
| | | YGGFMRGL |
| | Metorphamide | YGGFMRRV-NH₂ |
| Pro-dynorphin | Dynorphin A | YGGFLRPJRPKLKWDNQ |
| | Dynorphin A(1-8) | YGGFLRPJ |
| | Dynorphin B | YGGFLRRQFKWT |
| | α-neoendorphin | YGGFLRKYPK |
| | β-neoendorphin | YGGFLRKYP |
| Pro- | Nociceptin | FGGFTGARKSARKLANQ |
| nociceptin/OFQ | | |
| Pro-endomorphin* | Endomorphin-1 | YPWF-NH₂ |
| | Endomorphin-2 | YPFF-NH₂ |

Opioid receptor activation produces a wide array of cellular responses (Table 4). For example, there are Direct G-protein bg or a subunit-mediated effects, such as activation of an inwardly rectifying potassium channel, inhibition of voltage operated calcium channels (N, P, Q and R type), inhibition of adenylyl cyclase, Responses of unknown intermediate mechanism, activation of PLA2, activation of PLC b (possibly direct G protein bg subunit activation), activation of MAPKinase, activation of large conductance calcium-activated potassium channels, activation of L type voltage operated calcium channels, inhibition of T type voltage operated calcium channels, and direct inhibition of transmitter exocytosis. There are also responses in other effector pathways, such as activation of voltage-sensitive potassium channels (activation of PLA2), inhibition of M channels (activation of PLA2), inhibition of the hyperpolarisation-activated cation channel (Ih) (reduction in cAMP levels following inhibition of adenylyl cyclase), elevation of intracellular free calcium levels (activation of PLCb, activation of L type voltage operated calcium conductance), potentiation of NMDA currents (activation of protein kinase C), inhibition of transmitter release (inhibition of adenylyl cyclase, activation of potassium channels and inhibition of voltage operated calcium channels), decreases in neuronal excitability (activation of potassium channels), increases in neuronal firing rate (inhibition of inhibitory transmitter release—disinhibition), and changes in gene expression (long-term changes in adenylyl cyclase activity, elevation of intracellular calcium levels, activation of cAMP response element binding protein (CREB)).

2. Compositions for Treating Pain

Disclosed are constructs and vectors for expressing one or more opioid receptors in a cell, such as a nerve cell, such as a peripheral nerve cell. As discussed herein, opioid receptors are typically expressed in the spinal or supraspinal nerve cells, and the periphery typically do not express these receptors. The disclosed compositions and methods are designed to express the opioid receptors in nerve cells which are damaged or transmitting because of trauma, but which do not have endogenous opioid receptors or insufficient numbers of endogenous receptors to react to the endogenous opioid like molecules, typically in the periphery of the nerve cell. Thus, the expression of the opioid receptors in the nerve cell near the point of pain, will typically increase the amount of opioid receptors in this area and thus, increase the responsiveness to endogenous opioid like molecules. By expression of the opioid receptors, the sensation of pain can be reduced, not by administration of opioid analgesics, but rather by more efficient use of endogenous opioid like compounds. It is understood, however, that opioids, opioid like molecules, and/or other pain alleviating molecules can be added in addition to the disclosed opioid receptors.

Disclosed are methods wherein administration occurs in the intra-articular region of the jaw. The results shown herein demonstrated that intra-articular injection of FIV(lacZ) resulted in successful gene transfer to articular TMJ surfaces as well as the joint meniscus. Thus, disclosed are methods, wherein the administration of the disclosed vectors, results in delivery to the articular TMJ surfaces and the joint meniscus.

Nociceptive innervation to the temporomandibular joint (TMJ) is primarily provided by the auriculotemporal nerve of the mandibular division of the trigeminal nerve (Sessle & Wu, 1991). Aδ and C nerve fibers, whose cell bodies are located in the posterolateral part of the trigeminal ganglion (Yoshino et al., 1998), project distally and terminate as non-encapsulated free nerve endings dispersed throughout the posterolateral part of the TMJ capsule (Bernick, 1962; Thilander, 1964; Frommer & Monroe, 1966; Klineberg, 1971), the posterior band of the meniscus and the posterior attachment (Dressen et al., 1990; Kido et al., 1991, 1993; Wink et al., 1992). Transfer of anti-nociceptive genes to sensory trigeminal neurons innervating the orofacial region can be achieved after injection of lentiviral vectors at the painful site, such as the TMJ, resulting in their uptake by free nerve endings and retrograde transport to the sensory cells' nuclei. Previous studies demonstrated axonal retrograde transport of horseradish peroxidase from the TMJ to the central nervous system (Romfh et al., 1979; Carpa, 1987) including the trigeminal ganglia (Yoshino et al., 1998).

Disclosed are constructs capable of expressing any of the opioid receptor gene products.

Disclosed are constructs capable of expressing opioid receptors, such as the μ-opioid receptor gene product. The μ-opioid receptor construct allows for synthesis of μ-opioid receptor protein.

The μ-opioid receptor construct typically comprises three parts: 1) a promoter, 2) the μ-opioid receptor coding sequence, and 3) polyA tail. The poly A tail can be from the bovine growth hormone or any polyA tail including synthetic poly A tails. The Bovine growth hormone poly A tail carries elements that not only increase expression, but also increase stability of any gene construct. These three parts can be integrated into any vector delivery system, which is capable of transducing terminally differentiated cells, such as nerve cells.

The promoter can be any promoter, such as those discussed herein. It is understood as discussed herein that there are functional variants of opioid receptors, such as the μ-opioid receptor protein which can be made. In certain embodiments the promoter is going to be a cell specific promoter, such as a nerve cell specific promoter, such as the neuron specific enolase promoter. Other promoters are disclosed herein.

The promoter can be any promoter, such as those discussed herein. It is understood as discussed herein that there are functional variants of opioid receptors, such as the μ-opioid receptor protein which can be made. In certain embodiments the promoter is going to be a cell specific promoter, such as a nerve cell specific promoter, such as the neuron specific enolase promoter.

μ-opioid receptor cDNA can be obtained from the American Tissue Culture Collection. (American Tissue Culture Collection, Manassas, Va. 20110-2209; μ-opioid receptor ATCC#. Raynor K, et al., Characterization of the cloned human mu opioid receptor. J Pharmacol Exp Ther. 1995; 272:423-8.)

Also disclosed are constructs encoding for the human or mouse μ-opioid receptor, as well as the β-galactosidase reporter gene (lacZ).

Disclosed are nucleic acids comprising sequence encoding μ-opioid receptor. Also disclosed are nucleic acids, wherein the nucleic acid further comprises a promoter sequence, wherein the μ-opioid receptor has at least 80% identity to the sequence set forth in SEQ ID NO:2 or 4, wherein the μ-opioid receptor has at least 85% identity to the sequence set forth in SEQ ID NO: 1 or 3, wherein the μ-opioid receptor has at least 90% identity to the sequence set forth in SEQ ID NO: 1 or 3, wherein the μ-opioid receptor has at least 95% identity to the sequence set forth in SEQ ID NO:1 or 3, and/or wherein the μ-opioid receptor has the sequence set forth in SEQ ID NO: 1 or 3.

Also disclosed are vectors comprising the disclosed nucleic acids. Also disclosed are cells comprising the disclosed nucleic acids and vectors. Any cell can be targeted with the disclosed constructs. However, nerve cells, for example, are terminally differentiated. This means that they are no longer dividing. The state of a mature non-dividing nerve cell can define terminally differentiated cells. In terms of differentiated\stable transduction, nerve cells thus represent attractive targets because once DNA is integrated, there is a very low probability that it will not remain in the cell.

Also disclosed are non-human mammals comprising the disclosed nucleic acids, vectors, and cells disclosed herein.

Also disclosed are methods of providing μ-opioid receptor in a cell comprising transfecting the cell with the nucleic acids.

Also disclosed are method of delivering the disclosed compositions, wherein the transfection occurs in vitro or in vivo.

Disclosed are methods of making a transgenic organism comprising administering the disclosed nucleic acids, vectors and/or cells.

Disclosed are methods of making a transgenic organism comprising transfecting a lentiviral vector to the organism at during a perinatal stage of the organism's development. Stragtegies of producing genetically engineered pluripotent, such as embryonic, stem cells, can be performed with the disclosed compositions to produce engineered cells and organisms as dicussed herein. Furthermore by cloning strategies can be used to produce desried organisms, which carry one or more of the disclosed compositions.

Also disclosed are methods of treating a subject having pain comprising administering any of the disclosed compounds and compositions.

Delivery of the disclosed constructs to terminally differentiated cells is also disclosed. Disclosed is a pseudotyped feline immunodeficiency virus (FIV) for μ-opioid receptor delivery to terminally differentiated cells. Stable expression of the therapeutic gene aids prolonged expression, enhancing treatment efficacy and contributing to long-term therapeutic outcomes. The backbone FIV system has been shown to effectively incorporate, due to its lentiviral properties, the transgene of interest into the host's genome, allowing for stable gene expression (Poeschla et al., 1998). Disclosed herein is stable expression of the reporter gene lacZ in N2a cells, following perinatal systemic FIV(lacZ) administration.

In certain embodiments the constructs become an integrated product with the genome of the host. For example, lentiviruses, such as HIV and LIV, have the characteristic of transfecting the therapeutic gene into the host chromosome, thus forming an integrated product. In certain embodiments, the requirement is that the vectors allow for expression in the periphery of the cell, such as the nerve cell, and/or at or near the point of pain. The contrast to integrated products is episomal products which can also be produced using, for example, HSV and AV vectors. Thus, transient expression can be beneficial. The optimal time of expression is correlated with the amount of product produced and amount that is needed. For example, in certain embodiments, expression for at least 3, 4, 5, 6, 7, 8, 9, 10, 15, 20, 30, 45, 60, 90, 120, 150, or 180 days is desirable.

A model system for the study of these vectors is a mouse that is knockout mouse deficient in μ-opioid receptor.

Stable transduced are cells where a nucleic acid has been integrated into the cell genome.

3. Delivery of the Compositions to Cells

Delivery can be applied, in general, via local or systemic routes of administration. Local administration includes virus injection directly into the region or organ of interest, versus intravenous (IV) or intraperitoneal (IP) injections (systemic) aiming at viral delivery to multiple sites and organs via the blood circulation. Previous research on the effects of local administration demonstrated gene expression limited to the site/organ of the injection, which did not extend to the rest of the body (Daly et al., 1999a; Kordower et al., 1999). Furthermore, previous studies have demonstrated successful global gene transfer to multiple tissues and organs in rodents and primates following viral IV and IP injections (Daly et al., 1999b; Tarntal et al., 2001; McCormack et al., 2001; Lipschutz et al., 2001). Disclosed herein IP injection of FIV(lacZ) in mice of adult (3 months old) as well as of perinatal age (P4) resulted in global transfer and expression of the reporter gene lacZ in brain, liver, spleen and kidney. Also disclosed, the levels of expression achieved via IP injections were superior to those acquired following local administration directly into the liver.

There are a number of compositions and methods which can be used to deliver nucleic acids to cells, either in vitro or in vivo. These methods and compositions can largely be broken down into two classes: viral based delivery systems and non-viral based delivery systems. For example, the nucleic acids can be delivered through a number of direct delivery systems such as, electroporation, lipofection, calcium phosphate precipitation, plasmids, viral vectors, viral nucleic acids, phage nucleic acids, phages, cosmids, or via transfer of genetic material in cells or carriers such as cationic liposomes. Appropriate means for transfection, including viral vectors, chemical transfectants, or physico-mechanical methods such as electroporation and direct diffusion of DNA, are described by, for example, Wolff, J. A., et al., Science, 247, 1465-1468, (1990); and Wolff, J. A. Nature, 352, 815-818, (1991). Such methods are well known in the art and readily adaptable for use with the compositions and methods described herein. In certain cases, the methods will be modified to specifically function with large DNA molecules. Further, these methods can be used to target certain diseases and cell populations by using the targeting characteristics of the carrier.

a) Nucleic Acid Based Delivery Systems

Transfer vectors can be any nucleotide construction used to deliver genes into cells (e.g., a plasmid), or as part of a general strategy to deliver genes, e.g., as part of recombinant retrovirus or adenovirus (Ram et al. Cancer Res. 53:83-88, (1993)).

As used herein, plasmid or viral vectors are agents that transport the disclosed nucleic acids, such as the μ-opioid receptor construct into the cell without degradation and include a promoter yielding expression of the μ-opioid receptor encoding sequences in the cells into which it is delivered. In some embodiments the vectors for the μ-opioid receptor constructs are derived from either a virus, retrovirus, or lentivirus. Viral vectors can be, for example, Adenovirus, Adeno-associated virus, Herpes virus, Vaccinia virus, Polio virus, AIDS virus, neuronal trophic virus, Sindbis and other RNA viruses, including these viruses with the HIV backbone, and lentiviruses. Also preferred are any viral families which share the properties of these viruses which make them suitable for use as vectors. Retroviruses include Murine Maloney Leukemia virus, MMLV, and retroviruses that express the desirable properties of MMLV as a vector. Retroviral vectors are able to carry a larger genetic payload, i.e., a transgene, such as, the disclosed μ-opioid receptor constructs or marker gene, than other viral vectors, and for this reason are a commonly used vector. However, they are not as useful in non-proliferating cells. Adenovirus vectors are relatively stable and easy to work with, have high titers, and can be delivered in aerosol formulation, and can transfect non-dividing cells. Pox viral vectors are large and have several sites for inserting genes, they are thermostable and can be stored at room temperature. A preferred embodiment is a viral vector, which has been engineered so as to suppress the immune response of the host organism, elicited by the viral antigens. Preferred vectors of this type will carry coding regions for Interleukin 8 or 10.

Viral vectors can have higher transaction (ability to introduce genes) abilities than chemical or physical methods to introduce genes into cells. Typically, viral vectors contain, nonstructural early genes, structural late genes, an RNA polymerase III transcript, inverted terminal repeats necessary for replication and encapsidation, and promoters to control the transcription and replication of the viral genome. When engineered as vectors, viruses typically have one or more of the early genes removed and a gene or gene/promotor cassette is inserted into the viral genome in place of the removed viral DNA. Constructs of this type can carry up to about 8 kb of foreign genetic material. The necessary functions of the removed early genes are typically supplied by cell lines which have been engineered to express the gene products of the early genes in trans.

(1) Retroviral Vectors

A retrovirus is an animal virus belonging to the virus family of Retroviridae, including any types, subfamilies, genus, or tropisms. Retroviral vectors, in general, are described by Verma, I. M., Retroviral vectors for gene transfer. In Microbiology-1985, American Society for Microbiology, pp. 229-232, Washington, (1985), which is incorporated by reference herein. Examples of methods for using retroviral vectors for gene therapy are described in U.S. Pat. Nos. 4,868,116 and 4,980,286; PCT applications WO 90/02806 and WO 89/07136; and Mulligan, (Science 260:926-932 (1993)); the teachings of which are incorporated herein by reference.

A retrovirus is essentially a package which has packed into it nucleic acid cargo. The nucleic acid cargo carries with it a packaging signal, which ensures that the replicated daughter molecules will be efficiently packaged within the package coat. In addition to the package signal, there are a number of molecules which are needed in cis, for the replication, and packaging of the replicated virus. Typically a retroviral genome, contains the gag, pol, and env genes which are involved in the making of the protein coat. It is the gag, pol, and env genes which are typically replaced by the foreign DNA that it is to be transferred to the target cell. Retrovirus vectors typically contain a packaging signal for incorporation into the package coat, a sequence which signals the start of the gag transcription unit, elements necessary for reverse transcription, including a primer binding site to bind the tRNA primer of reverse transcription, terminal repeat sequences that guide the switch of RNA strands during DNA synthesis, a purine rich sequence 5′ to the 3′ LTR that serve as the priming site for the synthesis of the second strand of DNA synthesis, and specific sequences near the ends of the LTRs that enable the insertion of the DNA state of the retrovirus to insert into the host genome. The removal of the gag, pol, and env genes allows for about 8 kb of foreign sequence to be inserted into the viral genome, become reverse transcribed, and upon replication be packaged into a new retroviral particle. This amount of nucleic acid is sufficient for the delivery of a one to many genes depending on the size of each transcript. It is preferable to include either positive or negative selectable markers along with other genes in the insert.

Since the replication machinery and packaging proteins in most retroviral vectors have been removed (gag, pol, and env); the vectors are typically generated by placing them into a packaging cell line. A packaging cell line is a cell line which has been transfected or transformed with a retrovirus that contains the replication and packaging machinery, but lacks any packaging signal. When the vector carrying the DNA of choice is transfected into these cell lines, the vector containing the gene of interest is replicated and packaged into new retroviral particles, by the machinery provided in cis by the helper cell. The genomes for the machinery are not packaged because they lack the necessary signals.

(2) Adenoviral Vectors

The construction of replication-defective adenoviruses has been described (Berkner et al., J. Virology 61:1213-1220 (1987); Massie et al., Mol. Cell. Biol. 6:2872-2883 (1986); Haj-Ahmad et al., J. Virology 57:267-274 (1986); Davidson et al., J. Virology 61:1226-1239 (1987); Zhang “Generation and identification of recombinant adenovirus by liposome-mediated transfection and PCR analysis” BioTechniques 15:868-872 (1993)). The benefit of the use of these viruses as vectors is that they are limited in the extent to which they can spread to other cell types, since they can replicate within an initial infected cell, but are unable to form new infectious viral particles. Recombinant adenoviruses have been shown to achieve high efficiency gene transfer after direct, in vivo delivery to airway epithelium, hepatocytes, vascular endothelium, CNS parenchyma and a number of other tissue sites (Morsy, J. Clin. Invest 92:1580-1586 (1993); Kirshenbaum, J. Clin. Invest. 92:381-387 (1993); Roessler, J. Clin. Invest. 92:1085-1092 (1993); Moullier, Nature Genetics 4:154-159 (1993); La Salle, Science 259:988-990 (1993); Gomez-Foix, J. Biol. Chem. 267:25129-25134 (1992); Rich, Human Gene Therapy 4:461-476 (1993); Zabner, Nature Genetics 6:75-83 (1994); Guzman, Circulation Research 73:1201-1207 (1993); Bout, Human Gene Therapy 5:3-10 (1994); Zabner, Cell 75:207-216 (1993); Caillaud, Eur. J. Neuroscience 5:1287-1291 (1993); and Ragot, J. Gen. Virology 74:501-507 (1993)). Recombinant adenoviruses achieve gene transduction by binding to specific cell surface receptors, after which the virus is internalized by receptor-mediated endocytosis, in the same manner as wild type or replication-defective adenovirus (Chardonnet and Dales, Virology 40:462-477 (1970); Brown and Burlingham, J. Virology 12:386-396 (1973); Svensson and Persson, J. Virology 55:442-449 (1985); Seth, et al., J. Virol. 51:650-655 (1984); Seth, et al., Mol. Cell. Biol. 4:1528-1533 (1984); Varga et al., J. Virology 65:6061-6070 (1991); Wickham et al., Cell 73:309-319 (1993)).

A viral vector can be one based on an adenovirus which has had the E1 gene removed and these virions are generated in a cell line such as the human 293 cell line. In another preferred embodiment both the E1 and E3 genes are removed from the adenovirus genome.

(3) Adeno-Asscociated Viral Vectors

Another type of viral vector is based on an adeno-associated virus (AAV). This defective parvovirus is a preferred vector because it can infect many cell types and is nonpathogenic to humans. AAV type vectors can transport about 4 to 5 kb and wild type AAV is known to stably insert into chromosome 19. Vectors which contain this site specific integration property are preferred. An especially preferred embodiment of this type of vector is the P4.1 C vector produced by Avigen, San Francisco, Calif., which can contain the herpes simplex virus thymidine kinase gene, HSV-tk, and/or a marker gene, such as the gene encoding the green fluorescent protein, GFP.

In another type of AAV virus, the AAV contains a pair of inverted terminal repeats (ITRs) which flank at least one cassette containing a promoter which directs cell-specific expression operably linked to a heterologous gene. Heterologous in this context refers to any nucleotide sequence or gene which is not native to the AAV or B19 parvovirus.

Typically the AAV and B19 coding regions have been deleted, resulting in a safe, noncytotoxic vector. The AAV ITRs, or modifications thereof, confer infectivity and site-specific integration, but not cytotoxicity, and the promoter directs cell-specific expression. U.S. Pat. No. 6,261,834 is herein incorporated by reference for material related to the AAV vector.

The vectors of the present invention thus provide DNA molecules which are capable of integration into a mammalian chromosome without substantial toxicity.

The inserted genes in viral and retroviral usually contain promoters, and/or enhancers to help control the expression of the desired gene product. A promoter is generally a sequence or sequences of DNA that function when in a relatively fixed location in regard to the transcription start site. A promoter contains core elements required for basic interaction of RNA polymerase and transcription factors, and can contain upstream elements and response elements.

(4) Lentiviral Vectors

The vectors can be lentiviral vectors, including but not limited to, SIV vectors, HIV vectors or a hybrid construct of these vectors, including viruses with the HIV backbone. These vectors also include first, second and third generation lentiviruses. Third generation lentiviruses have lentiviral packaging genes split into at least 3 independent plasmids or constructs. Also vectors can be any viral family that shares the properties of these viruses which make them suitable for use as vectors. Lentiviral vectors are a special type of retroviral vector which are typically characterized by having a long incubation period for infection. Furthermore, lentiviral vectors can infect non-dividing cells. Lentiviral vectors are based on the nucleic acid backbone of a virus from the lentiviral family of viruses. Typically, a lentiviral vector contains the 5′ and 3′ LTR regions of a lentivirus, such as SIV and HIV. Lentiviral vectors also typically contain the Rev Responsive Element (RRE) of a lentivirus, such as SIV and HIV.

(a) Feline Immunodeficiency Viral Vectors

One type of vector that the disclosed constructs can be delivered in is the VSV-G pseudotyped Feline Immunodeficiency Virus system developed by Poeschla et al. (1998). This lentivirus has been shown to efficiently infect dividing, growth arrested as well as post-mitotic cells. Furthermore, due to its lentiviral properties, it allows for incorporation of the transgene into the host's genome, leading to stable gene expression. This is a 3-vector system, whereby each confers distinct instructions: the FIV vector carries the transgene of interest and lentiviral apparatus with mutated packaging and envelope genes. A vesicular stomatitis virus G-glycoprotein vector (VSV-G; Burns et al., 1993) contributes to the formation of the viral envelope in trans. The third vector confers packaging instructions in trans (Poeschla et al., 1998). FIV production is accomplished in vitro following co-transfection of the aforementioned vectors into 293-T cells. The FIV-rich supernatant is then collected, filtered and can be used directly or following concentration by centrifugation. Titers routinely range between 10⁴-10⁷ bfu/ml.

(5) Packaging Vectors

As discussed above, retroviral vectors are based on retroviruses which contain a number of different sequence elements that control things as diverse as integration of the virus, replication of the integrated virus, replication of un-integrated virus, cellular invasion, and packaging of the virus into infectious particles. While the vectors in theory could contain all of their necessary elements, as well as an exogenous gene element (if the exogenous gene element is small enough) typically many of the necessary elements are removed. Since all of the packaging and replication components have been removed from the typical retroviral, including lentiviral, vectors which will be used within a subject, the vectors need to be packaged into the initial infectious particle through the use of packaging vectors and packaging cell lines. Typically retroviral vectors have been engineered so that the myriad functions of the retrovirus are separated onto at least two vectors, a packaging vector and a delivery vector. This type of system then requires the presence of all of the vectors providing all of the elements in the same cell before an infectious particle can be produced. The packaging vector typically carries the structural and replication genes derived from the retrovirus, and the delivery vector is the vector that carries the exogenous gene element that is preferably expressed in the target cell. These types of systems can split the packaging functions of the packaging vector into multiple vectors, e.g., third-generation lentivirus systems. Dull, T. et al., “A Third-generation lentivirus vector with a conditional packaging system” J. Virol 72(11):8463-71 (1998)

Retroviruses typically contain an envelope protein (env). The Env protein is in essence the protein which surrounds the nucleic acid cargo. Furthermore cellular infection specificity is based on the particular Env protein associated with a typical retrovirus. In typical packaging vector/delivery vector systems, the Env protein is expressed from a separate vector than for example the protease (pro) or integrase (in) proteins.

(6) Packaging Cell Lines

The vectors are typically generated by placing them into a packaging cell line. A packaging cell line is a cell line which has been transfected or transformed with a retrovirus that contains the replication and packaging machinery, but lacks any packaging signal. When the vector carrying the DNA of choice is transfected into these cell lines, the vector containing the gene of interest is replicated and packaged into new retroviral particles, by the machinery provided in cis by the helper cell. The genomes for the machinery are not packaged because they lack the necessary signals. One type of packaging cell line is a 293 cell line.

(7) Large Payload Viral Vectors

Molecular genetic experiments with large human herpesviruses have provided a means whereby large heterologous DNA fragments can be cloned, propagated and established in cells permissive for infection with herpesviruses (Sun et al., Nature genetics 8: 33-41, 1994; Cotter and Robertson. Curr Opin Mol Ther 5: 633-644, 1999). These large DNA viruses (herpes simplex virus (HSV) and Epstein-Barr virus (EBV), have the potential to deliver fragments of human heterologous DNA>150 kb to specific cells. EBV recombinants can maintain large pieces of DNA in the infected B-cells as episomal DNA. Individual clones carried human genomic inserts up to 330 kb appeared genetically stable The maintenance of these episomes requires a specific EBV nuclear protein, EBNA1, constitutively expressed during infection with EBV. Additionally, these vectors can be used for transfection, where large amounts of protein can be generated transiently in vitro. Herpesvirus amplicon systems are also being used to package pieces of DNA>220 kb and to infect cells that can stably maintain DNA as episomes.

Other useful systems include, for example, replicating and host-restricted non-replicating vaccinia virus vectors.

b) Non-Nucleic Acid Based Systems

The disclosed compositions can be delivered to the target cells in a variety of ways. For example, the compositions can be delivered through electroporation, or through lipofection, or through calcium phosphate precipitation. The delivery mechanism chosen will depend in part on the type of cell targeted and whether the delivery is occurring for example in vivo or in vitro.

Thus, the compositions can comprise, in addition to the disclosed constructs or vectors for example, lipids such as liposomes, such as cationic liposomes (e.g., DOTMA, DOPE, DC-cholesterol) or anionic liposomes. Liposomes can further comprise proteins to facilitate targeting a particular cell, if desired. Administration of a composition comprising a compound and a cationic liposome can be administered to the blood afferent to a target organ or inhaled into the respiratory tract to target cells of the respiratory tract. Regarding liposomes, see, e.g., Brigham et al. Am. J. Resp. Cell. Mol. Biol. 1:95-100 (1989); Felgner et al. Proc. Natl. Acad. Sci USA 84:7413-7417 (1987); U.S. Pat. No. 4,897,355. Furthermore, the compound can be administered as a component of a microcapsule that can be targeted to specific cell types, such as macrophages, or where the diffusion of the compound or delivery of the compound from the microcapsule is designed for a specific rate or dosage.

In the methods described above which include the administration and uptake of exogenous DNA into the cells of a subject (i.e., gene transduction or transfection), delivery of the compositions to cells can be via a variety of mechanisms. As one example, delivery can be via a liposome, using commercially available liposome preparations such as LIPOFECTIN, LIPOFECTAMINE (GIBCO-BRL, Inc., Gaithersburg, Md.), SUPERFECT (Qiagen, Inc. Hilden, Germany) and TRANSFECTAM (Promega Biotec, Inc., Madison, Wis.), as well as other liposomes developed according to procedures standard in the art. In addition, the nucleic acid or vector of this invention can be delivered in vivo by electroporation, the technology for which is available from Genetronics, Inc. (San Diego, Calif.) as well as by means of a SONOPORATION machine (ImaRx Pharmaceutical Corp., Tucson, Ariz.).

The materials can be in solution, suspension (for example, incorporated into microparticles, liposomes, or cells). These can be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K. D., Br. J. Cancer, 60:275-281, (1989); Bagshawe, et al., Br. J. Cancer, 58:700-703, (1988); Senter, et al., Bioconjugate Chem., 4:3-9, (1993); Battelli, et al., Cancer Immunol. Immunother., 35:421-425, (1992); Pietersz and McKenzie, Immunolog. Reviews, 129:57-80, (1992); and Roffler, et al., Biochem. Pharmacol, 42:2062-2065, (1991)). These techniques can be used for a variety of other specific cell types. Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research. 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399-409 (1991)).

Nucleic acids that are delivered to cells which are to be integrated into the host cell genome, typically contain integration sequences. These sequences are often viral related sequences, particularly when viral based systems are used. These viral integration systems can also be incorporated into nucleic acids which are to be delivered using a non-nucleic acid based system of deliver, such as a liposome, so that the nucleic acid contained in the delivery system can be come integrated into the host genome.

Other general techniques for integration into the host genome include, for example, systems designed to promote homologous recombination with the host genome. These systems typically rely on sequence flanking the nucleic acid to be expressed that has enough homology with a target sequence within the host cell genome that recombination between the vector nucleic acid and the target nucleic acid takes place, causing the delivered nucleic acid to be integrated into the host genome. These systems and the methods necessary to promote homologous recombination are known to those of skill in the art

c) In Vivo/Ex Vivo

As described herein, the compositions can be administered in a pharmaceutically acceptable carrier and can be delivered to the subject's cells in vivo and/or ex vivo by a variety of mechanisms well known in the art (e.g., uptake of naked DNA, liposome fusion, intramuscular injection of DNA via a gene gun, endocytosis and the like).

If ex vivo methods are employed, cells or tissues can be removed and maintained outside the body according to standard protocols well known in the art. The compositions can be introduced into the cells via any gene transfer mechanism, such as, for example, calcium phosphate mediated gene delivery, electroporation, microinjection or proteoliposomes. The transduced cells can then be infused (e.g., in a pharmaceutically acceptable carrier) or homotopically transplanted back into the subject per standard methods for the cell or tissue type. Standard methods are known for transplantation or infusion of various cells into a subject.

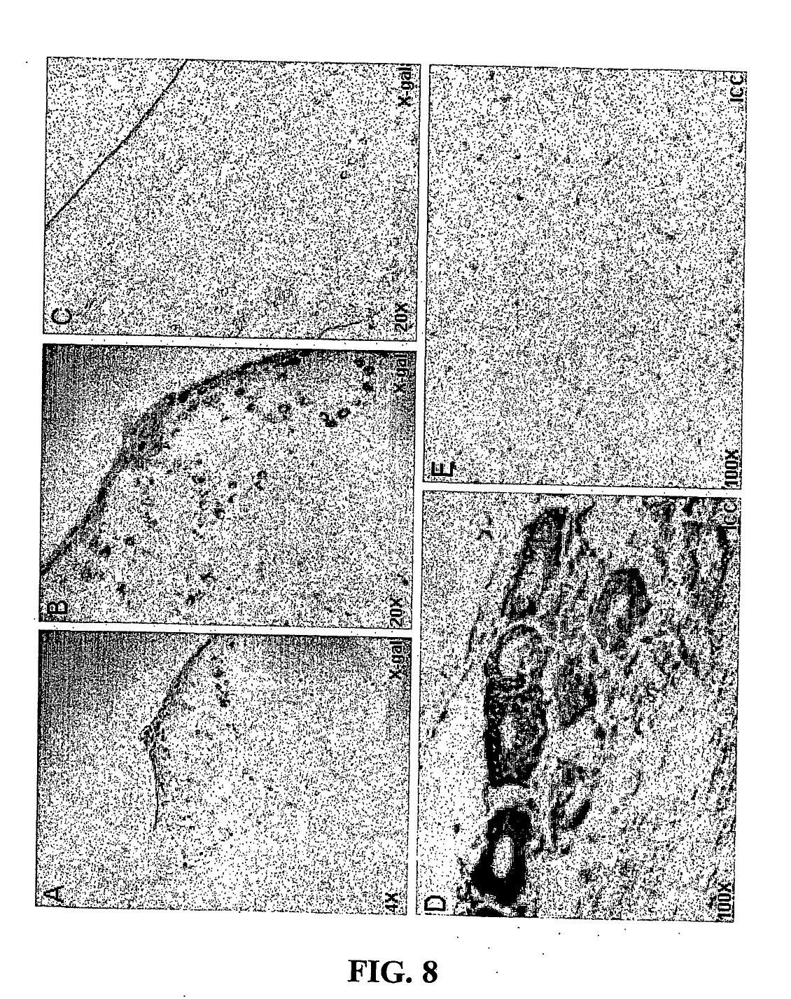

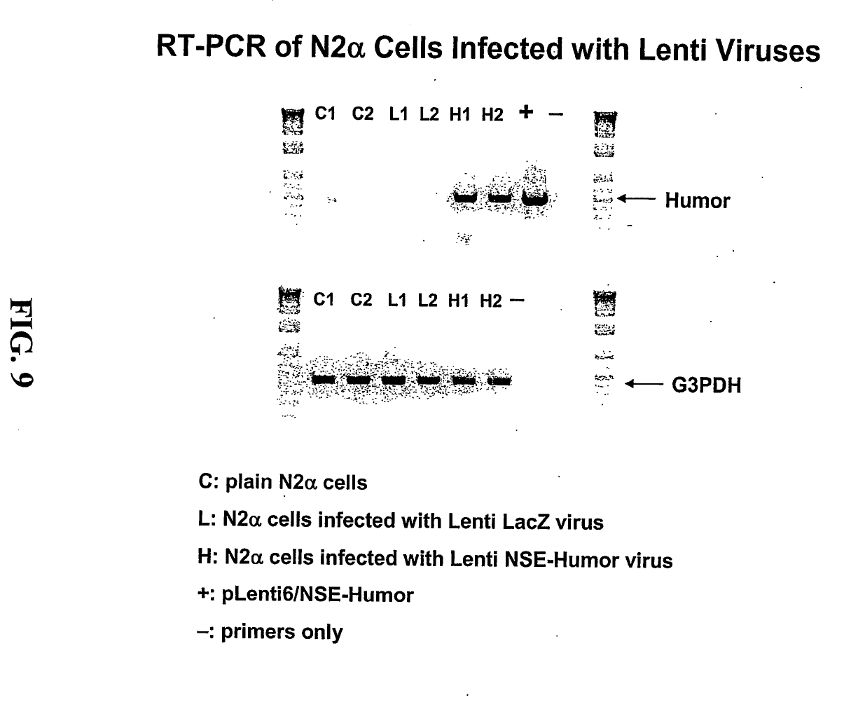

If in vivo delivery methods are performed the methods can be designed to deliver the nucleic acid constructs directly to a particular cell type, via any delivery mechanism, such as intra-peritoneal injection of a vector construct. In this type of delivery situation, the nucleic acid constructs can be delivered to any type of tissue, for example, brain or neural or muscle. The nucleic acid constructs can also be delivered such that they generally deliver the nucleic acid constructs to more than one type of cell. This type of delivery can be accomplished, by for example, injecting the constructs intraperitoneally into the flank of the organism. (See Example 2 and FIGS. 8-10). In certain delivery methods, the timing of the delivery is monitored. For example, the nucleic acid constructs can be delivered at the perinatal stage of the recipient's life or at the adult stage.

The various vectors delivering the opioid receptors, such as the m-opioid receptor can be delivered to differentiated cells. For example, cells that are quiescent can be targeted with the disclosed vectors in certain embodiments. For example, nerve cells, which are no longer dividing, or are dividing very slowly, can be transfected with the disclosed compositions in certain embodiments. The nucleic acids can be delivered peripherally in certain embodiments and can be delivered by injection at a site distal to the body of the cell. For example, pain may be initiated at a point in the foot of an organism, but the body of the nerve transmitting the pain signal will be located at or near the spinal cord. In certain embodiments, the compositions can be delivered at the foot, transfecting the distal part of the nerve, including the most distal part of the nerve. Transfection, can take place along the full length of the cell, however. In certain embodiments, the vectors are delivered by injection at a site distal to a nerve body, or, for example, at the point of the pain with regard to where the body of the nerve is located.

4. Expression Systems

The nucleic acids that are delivered to cells typically contain expression controlling systems. For example, the inserted genes in viral and retroviral systems usually contain promoters, and/or enhancers to help control the expression of the desired gene product. A promoter is generally a sequence or sequences of DNA that function when in a relatively fixed location in regard to the transcription start site. A promoter contains core elements required for basic interaction of RNA polymerase and transcription factors, and can contain upstream elements and response elements.

a) Promoters and Enhancers

Preferred promoters controlling transcription from vectors in mammalian host cells can be obtained from various sources, for example, the genomes of viruses such as: polyoma, Simian Virus 40 (SV40), adenovirus, retroviruses, hepatitis-B virus and most preferably cytomegalovirus, or from heterologous mammalian promoters, e.g. beta actin promoter. The early and late promoters of the SV40 virus are conveniently obtained as an SV40 restriction fragment which also contains the SV40 viral origin of replication (Fiers et al., Nature, 273: 113 (1978)). The immediate early promoter of the human cytomegalovirus is conveniently obtained as a HindIII E restriction fragment (Greenway, P. J. et al., Gene 18: 355-360 (1982)). Of course, promoters from the host cell or related species also are useful herein.

Enhancer generally refers to a sequence of DNA that functions at no fixed distance from the transcription start site and can be either 5′ (Laimins, L. et al., Proc. Natl. Acad. Sci. 78: 993 (1981)) or 3′ (Lusky, M. L., et al., Mol. Cell Bio. 3: 1108 (1983)) to the transcription unit. Furthermore, enhancers can be within an intron (Baneiji, J. L. et al., Cell 33: 729 (1983)) as well as within the coding sequence itself (Osborne, T. F., et al., Mol. Cell Bio. 4: 1293 (1984)). They are usually between 10 and 300 bp in length, and they function in cis. Enhancers function to increase transcription from nearby promoters. Enhancers also often contain response elements that mediate the regulation of transcription. Promoters can also contain response elements that mediate the regulation of transcription. Enhancers often determine the regulation of expression of a gene. While many enhancer sequences are now known from mammalian genes (globin, elastase, albumin, -fetoprotein and insulin), typically one will use an enhancer from a eukaryotic cell virus for general expression. Preferred examples are the SV40 enhancer on the late side of the replication origin (bp 100-270), the cytomegalovirus early promoter enhancer, the polyoma enhancer on the late side of the replication origin, and adenovirus enhancers.

The promoter and/or enhancer can be specifically activated either by light or specific chemical events which trigger their function. Systems can be regulated by reagents such as tetracycline and dexamethasone. There are also ways to enhance viral vector gene expression by exposure to irradiation, such as gamma irradiation, or alkylating chemotherapy drugs.

In certain embodiments the promoter and/or enhancer region can act as a constitutive promoter and/or enhancer to maximize expression of the region of the transcription unit to be transcribed. In certain constructs the promoter and/or enhancer region be active in all eukaryotic cell types, even if it is only expressed in a particular type of cell at a particular time. A preferred promoter of this type is the CMV promoter (650 bases). Other preferred promoters are SV40 promoters, cytomegalovirus (full length promoter), and retroviral vector LTF.

It has been shown that all specific regulatory elements can be cloned and used to construct expression vectors that are selectively expressed in specific cell types such as melanoma cells. The glial fibrillary acetic protein (GFAP) promoter has been used to selectively express genes in cells of glial origin.

Expression vectors used in eukaryotic host cells (yeast, fungi, insect, plant, animal, human or nucleated cells) can also contain sequences necessary for the termination of transcription which can affect mRNA expression. These regions are transcribed as polyadenylated segments in the untranslated portion of the mRNA encoding tissue factor protein. The 3′ untranslated regions also include transcription termination sites. It is preferred that the transcription unit also contains a polyadenylation region. One benefit of this region is that it increases the likelihood that the transcribed unit will be processed and transported like mRNA. The identification and use of polyadenylation signals in expression constructs is well established. It is preferred that homologous polyadenylation signals be used in the transgene constructs. In certain transcription units, the polyadenylation region is derived from the SV40 early polyadenylation signal and consists of about 400 bases. It is also preferred that the transcribed units contain other standard sequences alone or in combination with the above sequences improve expression from, or stability of, the construct.

b) Constitutive Promoters

In certain embodiments the promoters are constitutive promoters. This can be any promoter that causes transcription regulation in the absence of the addition of other factors. Examples of this type of promoter are the CMV promoter and the beta actin promoter, as well as others dicussed herein. In certain embodiments the promoter can consist of fusions of one or more different types of promoters. For example, the regulatory regions of the CMV promoter and the beta actin promoter are well known and understood, examples, of which are disclosed herein. Parts of these promoters can be fused together to, for example, produce a CMV-beta actin fusion promoter. It is understood that this type of promoter has a CMV component and a beta actin component. These components can function independently as promoters, and thus, are themselves considered beta actin promoters and CMV promoters. A promoter can be any portion of a known promoter that causes promoter activity. It is well understood that many promoters, including the CMV and Beta Actin promoters have functional domains which are understood and that these can be used as a beta actin promoter or CMV promoter. Furthermore, these domains can be determined. There are many CMV promoter variations that exist, as well as beta actin promoters, and fusion promoters. These promoters can be compared, and for example, functional regions delineated, as described herein. Furthermore, each of these sequences can function independently or together in any combination to provide a promoter region for the disclosed nucleic acids.

c) Non-Constitutive Promoters

The promoters can also be non-constitutive promoters, such as cell specific promoters. These are promoters that are turned on at specific time in development or stage or a particular type of cell, such as a cardiac cell, or neural cell, or a bone cell. Some examples of cell specific promoters are, the neural enolase specifc promoter, (NSE) the COLL1A1 procollagen promoter, and the CD11b promoter (PBMC-microglia/macrophage/monocyte specific promoter.

It is understood that the recombinant systems can be expressed in a tissue-specific manner. It is understood that tissue specific expression can occur due to the presence of a tissue-specific promoter. Typically, proteins under control of a tissue-specific promoter are transcribed when the promoter becomes active by virtue of being present in the tissue for which it is specific. Therefore, all cells can encode for a particular gene without global expression. As such, labeled proteins can be shown to be present in certain tissues without expression in other nearby tissues that may complicate results or expression of proteins in tissues where expression may be detrimental to the host. Disclosed are methods wherein the cre recombinase is under the control of the EIIA promoter, a promoter specific for breast tissue, such as the WAP promoter, a promoter specific for ovarian tissue, such as the ACTB promoter, or a promoter specific for bone tissue, such as osteocalcin. Any tissues specific promoter can be used. Promoters specific for prostate, testis, and neural are also disclosed. Examples of some tissue-specific promoters include but are not limited to MUC1, EIIA, ACTB, WAP, bHLH-EC2, HOXA-1, Alpha-fetoprotein (AFP), opsin, CR1/2, Fc-γ-Receptor 1 (Fc-γ-R1), MMTVD-LTR, the human insulin promoter, Pdha-2, rat neuron-specific enolase. For example, use of the AFP promoter creates specificity for the liver. Another example, HOXA-1 is a neuronal tissue specific promoter, and as such, proteins expressed under the control of HOXA-1 are only expressed in neuronal tissue. (All of which are herein incorporated by reference at least for the sequence of the promoters and related sequences.)

Other cell specific promoters can be found in (Sutcliffe, J. G. (1988), Ann. Rev. Neuroscience 11, 157-198). For example, when transfecting nerve cells, there are a variety of nerve specific promoters, such as the neuron specific enolase promoter. Other examples of neuron specific promoters would be the Tau promoter, Synapsin I (Hoesche, C., Sauerwald, A., et al., (1993) J. Biol. Chem. 268, 26494-26502 and II (Chin, L.-S et al., (1994), J. Biol. Chem. 269, 18507-18513) promoters, the amino acid decarboxylase (AADC) (Albert, V., et al., (1992), Proc. Natl. Acad. Sci. 89, 12053-12057) and FE65 (Faraonio, R., et al., (1994), Nucl. Acids Res. 22, 4876-4883) promoters. Other nerve specific promoters include, the promoter for the WT1 gene (Fraizer, G, et al., (1994), J. Biol. Chem. 269, 8892-8900), nuerofilament light chain promoter (Yazdanbakhsh, K., et al., (1993) Nucl. Acids Res. 21, 455-461), and the glial fibrillary acidic protein, (Kaneko, R. & Sueoka, N. (1993) Proc. Natl. Acad. Sci. 90, 4698-4702). (All of which are herein incorporated by reference at least for the sequence of the promoters and related sequences.)

Expression of the transgene can be targeted selectively to neurons by cloning a neuron specific promoter, such as the NSE promoter as disclosed herein (Liu H. et al., Journal of Neuroscience. 23(18):7143-54, 2003); tyrosine hydroxylase promoter (Kessler M A. et al., Brain Research. Molecular Brain Research. 112(1-2):8-23, 2003); myelin basic protein promoter (Kessler M A. et al Biochemical & Biophysical Research Communications. 288(4):809-18, 2001); glial fibrillary acidic protein promoter (Nolte C. et al., GLIA. 33(1):72-86, 2001); neurofilaments gene (heavy, medium, light) promoters (Yaworsky P J. et al., Journal of Biological Chemistry. 272(40):25112-20, 1997) (All of which are herein incorporated by reference at least for the sequence of the promoters and related sequences.) The NSE promoter is disclosed in Peel A L. et al., Gene Therapy. 4(1):16-24, 1997) (SEQ ID NO:69) (pTR-NT3myc; Powell Gene Therapy Center, University of Florida, Gainesville Fla.).

d) Markers

The viral vectors can include nucleic acid sequence encoding a marker product. This marker product is used to determine if the gene has been delivered to the cell and once delivered is being expressed. Preferred marker genes are the E. Coli lacZ gene, which encodes β-galactosidase, and green fluorescent protein.

In some embodiments the marker can be a selectable marker. Examples of suitable selectable markers for mammalian cells are dihydrofolate reductase (DHFR), thymidine kinase, neomycin, neomycin analog G418, hydromycin, and puromycin. When such selectable markers are successfully transferred into a mammalian host cell, the transformed mammalian host cell can survive if placed under selective pressure. There are two widely used distinct categories of selective regimes. The first category is based on a cell's metabolism and the use of a mutant cell line which lacks the ability to grow independent of a supplemented media. Two examples are: CHO DHFR-cells and mouse LTK-cells. These cells lack the ability to grow without the addition of such nutrients as thymidine or hypoxanthine. Because these cells lack certain genes necessary for a complete nucleotide synthesis pathway, they cannot survive unless the missing nucleotides are provided in a supplemented media. An alternative to supplementing the media is to introduce an intact DHFR or TK gene into cells lacking the respective genes, thus altering their growth requirements. Individual cells which were not transformed with the DHFR or TK gene will not be capable of survival in non-supplemented media.

The second category is dominant selection which refers to a selection scheme used in any cell type and does not require the use of a mutant cell line. These schemes typically use a drug to arrest growth of a host cell. Those cells which have a novel gene would express a protein conveying drug resistance and would survive the selection. Examples of such dominant selection use the drugs neomycin, (Southern P. and Berg, P., J. Molec. Appl. Genet. 1: 327 (1982)), mycophenolic acid, (Mulligan, R. C. and Berg, P. Science 209: 1422 (1980)) or hygromycin, (Sugden, B. et al., Mol. Cell. Biol. 5: 410-413 (1985)). The three examples employ bacterial genes under eukaryotic control to convey resistance to the appropriate drug G418 or neomycin (geneticin), xgpt (mycophenolic acid) or hygromycin, respectively. Others include the neomycin analog G418 and puramycin.

e) Post Transcriptional Regulatory Elements

The disclosed vectors can also contain post-transcriptional regulatory elements. Post-transcriptional regulatory elements can enhance mRNA stability or enhance translation of the transcribed mRNA. An exemplary post-transcriptional regulatory sequence is the WPRE sequence isolated from the woodchuck hepatitis virus. (Zufferey R, et al., “Woodchuck hepatitis virus post-transcriptional regulatory element enhances expression of transgenes delivered by retroviral vectors,” J Virol; 73:2886-92 (1999)). Post-transcriptional regulatory elements can be positioned both 3′ and 5′ to the exogenous gene, but it is preferred that they are positioned 3′ to the exogenous gene.

f) Transduction Efficiency Elements

Transduction efficiency elements are sequences that enhance the packaging and transduction of the vector. These elements typically contain polypurine sequences. An example of a transduction efficiency element is the ppt-cts sequence that contains the central polypurine tract (ppt) and central terminal site (cts) from the FIV. These sequences are in the disclosed FIV sequences herein. Each retrovirus and lentivirus can have there own ppt-cts.

g) 3′ Untranslated Regions

Expression vectors used in eukaryotic host cells (yeast, fungi, insect, plant, animal, human or nucleated cells) can also contain sequences necessary for the termination of transcription which can affect mRNA expression. These 3′ untranslated regions are transcribed as polyadenylated segments in the untranslated portion of the mRNA encoding the exogenous gene. The 3′ untranslated regions also include transcription termination sites. The transcription unit also can contain a polyadenylation region. One benefit of this region is that it increases the likelihood that the transcribed unit will be processed and transported like mRNA. The identification and use of polyadenylation signals in expression constructs is well established. Homologous polyadenylation signals can be used in the transgene constructs. In an embodiment of the transcription unit, the polyadenylation region is derived from the SV40 early polyadenylation signal and consists of about 400 bases. Transcribed units can contain other standard sequences alone or in combination with the above sequences improve expression from, or stability of, the construct.

5. Sequence Similarities

It is understood that as discussed herein the use of the terms homology and identity mean the same thing as similarity. Thus, for example, if the use of the word homology is used between two non-natural sequences it is understood that this is not necessarily indicating an evolutionary relationship between these two sequences, but rather is looking at the similarity or relatedness between their nucleic acid sequences. Many of the methods for determining homology between two evolutionarily related molecules are routinely applied to any two or more nucleic acids or proteins for the purpose of measuring sequence similarity regardless of whether they are evolutionarily related or not.

In general, it is understood that one way to define any known variants and derivatives or those that might arise, of the disclosed genes and proteins herein, is through defining the variants and derivatives in terms of homology to specific known sequences. This identity of particular sequences disclosed herein is also discussed elsewhere herein. In general, variants of genes and proteins herein disclosed typically have at least, about 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99 percent homology to the stated sequence or the native sequence. Those of skill in the art readily understand how to determine the homology of two proteins or nucleic acids, such as genes. For example, the homology can be calculated after aligning the two sequences so that the homology is at its highest level.

Another way of calculating homology can be performed by published algorithms. Optimal alignment of sequences for comparison can be conducted by the local homology algorithm of Smith and Waterman Adv. Appl. Math. 2: 482 (1981), by the homology alignment algorithm of Needleman and Wunsch, J. Mol. Biol. 48: 443 (1970), by the search for similarity method of Pearson and Lipman, Proc. Natl. Acad. Sci. U.S.A. 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by inspection.

The same types of homology can be obtained for nucleic acids by for example the algorithms disclosed in Zuker, M. Science 244:48-52, 1989, Jaeger et al. Proc. Natl. Acad. Sci. USA 86:7706-7710, 1989, Jaeger et al. Methods Enzymol. 183:281-306, 1989 which are herein incorporated by reference for at least material related to nucleic acid alignment. It is understood that any of the methods typically can be used and that in certain instances the results of these various methods can differ, but the skilled artisan understands if identity is found with at least one of these methods, the sequences would be said to have the stated identity, and be disclosed herein.

For example, as used herein, a sequence recited as having a particular percent homology to another sequence refers to sequences that have the recited homology as calculated by any one or more of the calculation methods described above. For example, a first sequence has 80 percent homology, as defined herein, to a second sequence if the first sequence is calculated to have 80 percent homology to the second sequence using the Zuker calculation method even if the first sequence does not have 80 percent homology to the second sequence as calculated by any of the other calculation methods. As another example, a first sequence has 80 percent homology, as defined herein, to a second sequence if the first sequence is calculated to have 80 percent homology to the second sequence using both the Zuker calculation method and the Pearson and Lipman calculation method even if the first sequence does not have 80 percent homology to the second sequence as calculated by the Smith and Waterman calculation method, the Needleman and Wunsch calculation method, the Jaeger calculation methods, or any of the other calculation methods. As yet another example, a first sequence has 80 percent homology, as defined herein, to a second sequence if the first sequence is calculated to have 80 percent homology to the second sequence using each of calculation methods (although, in practice, the different calculation methods will often result in different calculated homology percentages).

6. Hybridization/Selective Hybridization

The term hybridization typically means a sequence driven interaction between at least two nucleic acid molecules, such as a primer or a probe and a gene. Sequence driven interaction means an interaction that occurs between two nucleotides or nucleotide analogs or nucleotide derivatives in a nucleotide specific manner. For example, G interacting with C or A interacting with T are sequence driven interactions. Typically sequence driven interactions occur on the Watson-Crick face or Hoogsteen face of the nucleotide. The hybridization of two nucleic acids is affected by a number of conditions and parameters known to those of skill in the art. For example, the salt concentrations, pH, and temperature of the reaction all affect whether two nucleic acid molecules will hybridize.

Parameters for selective hybridization between two nucleic acid molecules are well known to those of skill in the art. For example, in some embodiments selective hybridization conditions can be defined as stringent hybridization conditions. For example, stringency of hybridization is controlled by both temperature and salt concentration of either or both of the hybridization and washing steps. For example, the conditions of hybridization to achieve selective hybridization can involve hybridization in high ionic strength solution (6×SSC or 6×SSPE) at a temperature that is about 12-25° C. below the Tm (the melting temperature at which half of the molecules dissociate from their hybridization partners) followed by washing at a combination of temperature and salt concentration chosen so that the washing temperature is about 5° C. to 20° C. below the Tm. The temperature and salt conditions are readily determined empirically in preliminary experiments in which samples of reference DNA immobilized on filters are hybridized to a labeled nucleic acid of interest and then washed under conditions of different stringencies. Hybridization temperatures are typically higher for DNA-RNA and RNA-RNA hybridizations. The conditions can be used as described above to achieve stringency, or as is known in the art. (Sambrook et al., Molecular Cloning: A Laboratory Manual, 2nd Ed., Cold Spring Harbor Laboratory, Cold Spring Harbor, N.Y., 1989; Kunkel et al. Methods Enzymol. 1987:154:367, 1987 which is herein incorporated by reference for material at least related to hybridization of nucleic acids). A preferable stringent hybridization condition for a DNA:DNA hybridization can be at about 68° C. (in aqueous solution) in 6×SSC or 6×SSPE followed by washing at 68° C. Stringency of hybridization and washing, if desired, can be reduced accordingly as the degree of complementarity desired is decreased, and further, depending upon the G-C or A-T richness of any area wherein variability is searched for. Likewise, stringency of hybridization and washing, if desired, can be increased accordingly as homology desired is increased, and further, depending upon the G-C or A-T richness of any area wherein high homology is desired, all as known in the art.

Another way to define selective hybridization is by looking at the amount (percentage) of one of the nucleic acids bound to the other nucleic acid. For example, in some embodiments selective hybridization conditions would be when at least about, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the limiting nucleic acid is bound to the non-limiting nucleic acid. Typically, the non-limiting primer is in for example, 10 or 100 or 1000 fold excess. This type of assay can be performed at under conditions where both the limiting and non-limiting primer are for example, 10 fold or 100-fold or 1000 fold below their k_{d}, or where only one of the nucleic acid molecules is 10 fold or 100 fold or 1000 fold or where one or both nucleic acid molecules are above their k_{d}.

Another way to define selective hybridization is by looking at the percentage of primer that gets enzymatically manipulated under conditions where hybridization is required to promote the desired enzymatic manipulation. For example, in some embodiments selective hybridization conditions would be when at least about, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the primer is enzymatically manipulated under conditions which promote the enzymatic manipulation, for example if the enzymatic manipulation is DNA extension, then selective hybridization conditions would be when at least about 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the primer molecules are extended. Preferred conditions also include those suggested by the manufacturer or indicated in the art as being appropriate for the enzyme performing the manipulation.

Just as with homology, it is understood that there are a variety of methods herein disclosed for determining the level of hybridization between two nucleic acid molecules. It is understood that these methods and conditions can provide different percentages of hybridization between two nucleic acid molecules, but unless otherwise indicated meeting the parameters of any of the methods would be sufficient. For example if 80% hybridization was required and as long as hybridization occurs within the required parameters in any one of these methods it is considered disclosed herein.

It is understood that those of skill in the art understand that if a composition or method meets any one of these criteria for determining hybridization either collectively or singly it is a composition or method that is disclosed herein.

7. Nucleic Acids

There are a variety of molecules disclosed herein that are nucleic acid based, including for example the nucleic acids that encode, for example μ-opioid receptor, or functional nucleic acids. The disclosed nucleic acids can be made up of for example, nucleotides, nucleotide analogs, or nucleotide substitutes. Non-limiting examples of these and other molecules are discussed herein. It is understood that for example, when a vector is expressed in a cell, that the expressed mRNA will typically be made up of A, C, G, and U. Likewise, it is understood that if, for example, an antisense molecule is introduced into a cell or cell environment through for example exogenous delivery, it is advantagous that the antisense molecule be made up of nucleotide analogs that reduce the degradation of the antisense molecule in the cellular environment

A nucleotide is a molecule that contains a base moiety, a sugar moiety and a phosphate moiety. Nucleotides can be linked together through their phosphate moieties and sugar moieties creating an internucleoside linkage. The base moiety of a nucleotide can be adenin-9-yl (A), cytosin-1-yl (C), guanin-9-yl (G), uracil-1-yl (U), and thymin-1-yl (T). The sugar moiety of a nucleotide is a ribose or a deoxyribose. The phosphate moiety of a nucleotide is pentavalent phosphate. A non-limiting example of a nucleotide would be 3′-AMP (3′-adenosine monophosphate) or 5′-GMP (5′-guanosine monophosphate).

A nucleotide analog is a nucleotide which contains some type of modification to either the base, sugar, or phosphate moieties. Modifications to nucleotides are well known in the art and would include for example, 5-methylcytosine (5-me-C), 5-hydroxymethyl cytosine, xanthine, hypoxanthine, and 2-aminoadenine as well as modifications at the sugar or phosphate moieties.

Nucleotide substitutes are molecules having similar functional properties to nucleotides, but which do not contain a phosphate moiety, such as peptide nucleic acid (DNA). Nucleotide substitutes are molecules that will recognize nucleic acids in a Watson-Crick or Hoogsteen manner, but which are linked together through a moiety other than a phosphate moiety. Nucleotide substitutes are able to conform to a double helix type structure when interacting with the appropriate target nucleic acid.

It is also possible to link other types of molecules (conjugates) to nucleotides or nucleotide analogs to enhance for example, cellular uptake. Conjugates can be chemically linked to the nucleotide or nucleotide analogs. Such conjugates include but are not limited to lipid moieties such as a cholesterol moiety. (Letsinger et al., Proc. Natl. Acad. Sci. USA, 1989, 86, 6553-6556),

A Watson-Crick interaction is at least one interaction with the Watson-Crick face of a nucleotide, nucleotide analog, or nucleotide substitute. The Watson-Crick face of a nucleotide, nucleotide analog, or nucleotide substitute includes the C2, N1, and C6 positions of a purine based nucleotide, nucleotide analog, or nucleotide substitute and the C2, N3, C4 positions of a pyrimidine based nucleotide, nucleotide analog, or nucleotide substitute.

A Hoogsteen interaction is the interaction that takes place on the Hoogsteen face of a nucleotide or nucleotide analog, which is exposed in the major groove of duplex DNA. The Hoogsteen face includes the N7 position and reactive groups (NH2 or O) at the C6 position of purine nucleotides.

a) Sequences

There are a variety of sequences related to μ-opioid receptor and promoter sequences. These sequences and others are herein incorporated by reference in their entireties as well as for individual subsequences contained therein. It is understood that there are numerous Genbank accession sequences related to μ-opioid receptor, all of which are incorporated by reference herein.

One particular sequence set forth in SEQ ID NO:2, which is a sequence for human μ-opioid receptor cDNA, is used herein, as an example, to exemplify the disclosed compositions and methods. It is understood that the description related to this sequence is applicable to any sequence related to μ-opioid receptor unless specifically indicated otherwise. Those of skill in the art understand how to resolve sequence discrepancies and differences and to adjust the compositions and methods relating to a particular sequence to other related sequences. Primers and/or probes can be designed for any of the sequences disclosed herein given the information disclosed herein and that known in the art.

It is also understood for example that there are numerous vectors that can be used to create the μ-opioid receptor construct nucleic acids.

b) Primers and Probes

Disclosed are compositions including primers and probes, which are capable of interacting with, for example, the μ-opioid receptor construct nucleic acids, as disclosed herein. In certain embodiments the primers are used to support DNA amplification reactions. Typically the primers will be capable of being extended in a sequence specific manner. Extension of a primer in a sequence specific manner includes any methods wherein the sequence and/or composition of the nucleic acid molecule to which the primer is hybridized or otherwise associated directs or influences the composition or sequence of the product produced by the extension of the primer. Extension of the primer in a sequence specific manner therefore includes, but is not limited to, PCR, DNA sequencing, DNA extension, DNA polymerization, RNA transcription, or reverse transcription. Techniques and conditions that amplify the primer in a sequence specific manner are preferred. In certain embodiments the primers are used for the DNA amplification reactions, such as PCR or direct sequencing. It is understood that in certain embodiments the primers can also be extended using non-enzymatic techniques, where for example, the nucleotides or oligonucleotides used to extend the primer are modified such that they will chemically react to extend the primer in a sequence specific manner. Typically the disclosed primers hybridize with, for example, the μ-opioid receptor construct nucleic acid, or region of the μ-opioid receptor construct nucleic acids or they hybridize with the complement of the μ-opioid receptor construct nucleic acids or complement of a region of the μ-opioid receptor construct nucleic acids.

8. Peptides

a) Protein Variants

As discussed herein there are numerous variants of the μ-opioid receptor protein that are known and herein contemplated. In addition, to the known functional species and allelic variants of μ-opioid receptor there are derivatives of the μ-opioid receptor protein which also function in the disclosed methods and compositions. Protein variants and derivatives are well understood to those of skill in the art and in can involve amino acid sequence modifications. For example, amino acid sequence modifications typically fall into one or more of three classes: substitutional, insertional or deletional variants. Insertions include amino and/or carboxyl terminal fusions as well as intrasequence insertions of single or multiple amino acid residues. Insertions ordinarily will be smaller insertions than those of amino or carboxyl terminal fusions, for example, on the order of one to four residues. Immunogenic fusion protein derivatives, such as those described in the examples, are made by fusing a polypeptide sufficiently large to confer immunogenicity to the target sequence by cross-linking in vitro or by recombinant cell culture transformed with DNA encoding the fusion. Deletions are characterized by the removal of one or more amino acid residues from the protein sequence. Typically, no more than about from 2 to 6 residues are deleted at any one site within the protein molecule. These variants ordinarily are prepared by site specific mutagenesis of nucleotides in the DNA encoding the protein, thereby producing DNA encoding the variant, and thereafter expressing the DNA in recombinant cell culture. Techniques for making substitution mutations at predetermined sites in DNA having a known sequence are well known, for example M13 primer mutagenesis and PCR mutagenesis. Amino acid substitutions are typically of single residues, but can occur at a number of different locations at once; insertions usually will be on the order of about from 1 to 10 amino acid residues; and deletions will range about from 1 to 30 residues. Deletions or insertions preferably are made in adjacent pairs, i.e. a deletion of 2 residues or insertion of 2 residues. Substitutions, deletions, insertions or any combination thereof can be combined to arrive at a final construct. The mutations must not place the sequence out of reading frame and preferably will not create complementary regions that could produce secondary mRNA structure. Substitutional variants are those in which at least one residue has been removed and a different residue inserted in its place. Such substitutions generally are made in accordance with the following Tables 4 and 5 and are referred to as conservative substitutions.

TABLE 4
Amino Acid Abbreviations

| Amino Acid | Abbreviations |
|---|---|
| alanine | AlaA |
| allosoleucine | Aile |
| arginine | ArgR |
| asparagine | AsnN |
| aspartic acid | AspD |
| cysteine | CysC |
| glutamic acid | GluE |
| glutamine | GlnK |
| glycine | GlyG |
| histidine | HisH |
| isolelucine | IleI |
| leucine | LeuL |
| lysine | LysK |
| phenylalanine | PheF |
| proline | ProP |
| pyroglutamic | Glu |
| acidp | |
| serine | SerS |
| threonine | ThrT |
| tyrosine | TyrY |
| tryptophan | TrpW |
| valine | ValV |

TABLE 5
Amino Acid Substitutions
Original Residue Exemplary Conservative Substitutions,
others are known in the art.

| | |
|---|---|
| Ala | ser |
| Arg | lys, gln |
| Asn | gln; his |
| Asp | glu |
| Cys | ser |
| Gln | asn, lys |
| Glu | asp |
| Gly | pro |
| His | asn; gln |
| Ile | leu; val |
| Leu | ile; val |
| Lys | arg; gln; |
| Met | Leu; ile |
| Phe | met; leu; tyr |
| Ser | thr |
| Thr | ser |
| Trp | tyr |
| Tyr | trp; phe |
| Val | ile; leu |

Substantial changes in function or immunological identity are made by selecting substitutions that are less conservative than those in Table 2, i.e., selecting residues that differ more significantly in their effect on maintaining (a) the structure of the polypeptide backbone in the area of the substitution, for example as a sheet or helical conformation, (b) the charge or hydrophobicity of the molecule at the target site or (c) the bulk of the side chain. The substitutions which in general are expected to produce the greatest changes in the protein properties will be those in which (a) a hydrophilic residue, e.g. seryl or threonyl, is substituted for (or by) a hydrophobic residue, e.g. leucyl, isoleucyl, phenylalanyl, valyl or alanyl; (b) a cysteine or proline is substituted for (or by) any other residue; (c) a residue having an electropositive side chain, e.g., lysyl, arginyl, or histidyl, is substituted for (or by) an electronegative residue, e.g., glutamyl or aspartyl; or (d) a residue having a bulky side chain, e.g., phenylalanine, is substituted for (or by) one not having a side chain, e.g., glycine, in this case, (e) by increasing the number of sites for sulfation and/or glycosylation.

For example, the replacement of one amino acid residue with another that is biologically and/or chemically similar is known to those skilled in the art as a conservative substitution. For example, a conservative substitution would be replacing one hydrophobic residue for another, or one polar residue for another. The substitutions include combinations such as, for example, Gly, Ala; Val, Ile, Leu; Asp, Glu; Asn, Gln; Ser, Thr, Lys, Arg; and Phe, Tyr. Such conservatively substituted variations of each explicitly disclosed sequence are included within the mosaic polypeptides provided herein.

Substitutional or deletional mutagenesis can be employed to insert sites for N-glycosylation (Asn-X-Thr/Ser) or O-glycosylation (Ser or Thr). Deletions of cysteine or other labile residues also may be desirable. Deletions or substitutions of potential proteolysis sites, e.g. Arg, is accomplished for example by deleting one of the basic residues or substituting one by glutaminyl or histidyl residues.

Certain post-translational derivatizations are the result of the action of recombinant host cells on the expressed polypeptide. Glutaminyl and asparaginyl residues are frequently post-translationally deamidated to the corresponding glutamyl and asparyl residues. Alternatively, these residues are deamidated under mildly acidic conditions. Other post-translational modifications include hydroxylation of proline and lysine, phosphorylation of hydroxyl groups of seryl or threonyl residues, methylation of the o-amino groups of lysine, arginine, and histidine side chains (T. E. Creighton, Proteins: Structure and Molecular Properties, W.H. Freeman & Co., San Francisco pp 79-86 (1983)), acetylation of the N-terminal amine and, in some instances, amidation of the C-terminal carboxyl.

It is understood that one way to define the variants and derivatives of the disclosed proteins herein is through defining the variants and derivatives in terms of homology/identity to specific known sequences. For example, SEQ ID NO: 1 sets forth a particular sequence of μ-opioid receptor. Specifically disclosed are variants of these and other proteins herein disclosed which have at least, 70% or 75% or 80% or 85% or 90% or 95% homology to the stated sequence. Those of skill in the art readily understand how to determine the homology of two proteins. For example, the homology can be calculated after aligning the two sequences so that the homology is at its highest level.

Another way of calculating homology can be performed by published algorithms. Optimal alignment of sequences for comparison can be conducted by the local homology algorithm of Smith and Waterman Adv. Appl. Math. 2: 482 (1981), by the homology alignment algorithm of Needleman and Wunsch, J. Mol. Biol. 48: 443 (1970), by the search for similarity method of Pearson and Lipman, Proc. Natl. Acad. Sci. U.S.A. 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis., or by inspection.

The same types of homology can be obtained for nucleic acids by for example the algorithms disclosed in Zuker, M. Science 244:48-52, 1989, Jaeger et al. Proc. Natl. Acad. Sci. USA 86:7706-7710, 1989, Jaeger et al. Methods Enzymol. 183:281-306, 1989 which are herein incorporated by reference for at least material related to nucleic acid alignment.

It is understood that the description of conservative mutations and homology can be combined together in any combination, such as embodiments that have at least 70% homology to a particular sequence wherein the variants are conservative mutations.

As this specification discusses various proteins and protein sequences it is understood that the nucleic acids that can encode those protein sequences are also disclosed. This would include all degenerate sequences related to a specific protein sequence, i.e. all nucleic acids having a sequence that encodes one particular protein sequence as well as all nucleic acids, including degenerate nucleic acids, encoding the disclosed variants and derivatives of the protein sequences. Thus, while each particular nucleic acid sequence may not be written out herein, it is understood that each and every sequence is in fact disclosed and described herein through the disclosed protein sequence. For example, one of the many nucleic acid sequences that can encode the protein sequence set forth in SEQ ID NO:3 is set forth in SEQ ID NO:4 Another nucleic acid sequence that encodes the same protein sequence set forth in SEQ ID NO:3 is set forth in SEQ ID NO:8. In addition, for example, a disclosed conservative derivative of SEQ ID NO:3 is shown in SEQ ID NO: 9, where the valine (V) at position 21 is changed to an isoleucine (I). It is understood that for this mutation, all of the nucleic acid sequences that encode this particular derivative of the SEQ ID NO:3 polypeptide are also disclosed. It is also understood that while no amino acid sequence indicates what particular DNA sequence encodes that protein within an organism, where particular variants of a disclosed protein are disclosed herein, the known nucleic acid sequence that encodes that protein in the particular organism from which that protein arises is also known and herein disclosed and described.

It is understood that there are numerous amino acid and peptide analogs which can be incorporated into the disclosed compositions. For example, there are numerous D amino acids or amino acids which have a different functional substituent then the amino acids shown in Table 1 and Table 2. The opposite stereo isomers of naturally occurring peptides are disclosed, as well as the stereo isomers of peptide analogs. These amino acids can readily be incorporated into polypeptide chains by charging tRNA molecules with the amino acid of choice and engineering genetic constructs that utilize, for example, amber codons, to insert the analog amino acid into a peptide chain in a site specific way (Thorson et al., Methods in Molec. Biol. 77:43-73 (1991), Zoller, Current Opinion in Biotechnology, 3:348-354 (1992); Ibba, Biotechnology & Genetic Enginerring Reviews 13:197-216 (1995), Cahill et al., TIBS, 14(10):400-403 (1989); Benner, TIB Tech, 12:158-163 (1994); Ibba and Hennecke, Bio/technology, 12:678-682 (1994) all of which are herein incorporated by reference at least for material related to amino acid analogs).

Molecules can be produced that resemble peptides, but which are not connected via a natural peptide linkage. For example, linkages for amino acids or amino acid analogs can include CH₂NH—, —CH₂S—, —CH₂—CH₂—, —CH═CH— (cis and trans), —COCH₂—, —CH(OH)CH₂—, and —CHH₂SO— (These and others can be found in Spatola, A. F. in Chemistry and Biochemistry of Amino Acids, Peptides, and Proteins, B. Weinstein, eds., Marcel Dekker, New York, p. 267 (1983); Spatola, A. F., Vega Data (March 1983), Vol. 1, Issue 3, Peptide Backbone Modifications (general review); Morley, Trends Pharm Sci (1980) pp. 463-468; Hudson, D. et al., Int J Pept Prot Res 14:177-185 (1979) (—CH₂NH—, CH₂CH₂—); Spatola et al. Life Sci 38:1243-1249 (1986) (—CHH₂—S); Hann J. Chem. Soc Perkin Trans. I 307-314 (1982) (—CH—CH—, cis and trans); Almquist et al. J. Med. Chem. 23:1392-1398 (1980) (—COCH₂—); Jennings-White et al. Tetrahedron Lett 23:2533 (1982) (—COCH₂—); Szelke et al. European Appln, EP 45665 CA (1982): 97:39405 (1982) (—CH(OH)CH₂—); Holladay et al. Tetrahedron. Lett 24:4401-4404 (1983) (—C(OH)CH₂—); and Hruby Life Sci 31:189-199 (1982) (—CH₂—S—); each of which is incorporated herein by reference. A particularly preferred non-peptide linkage is —CH₂NH—. It is understood that peptide analogs can have more than one atom between the bond atoms, such as b-alanine, g-aminobutyric acid, and the like.

Amino acid analogs and analogs and peptide analogs often have enhanced or desirable properties, such as, more economical production, greater chemical stability, enhanced pharmacological properties (half-life, absorption, potency, efficacy, etc.), altered specificity (e.g., a broad-spectrum of biological activities), reduced antigenicity, and others.

D-amino acids can be used to generate more stable peptides, because D amino acids are not recognized by peptidases and such. Systematic substitution of one or more amino acids of a consensus sequence with a D-amino acid of the same type (e.g., D-lysine in place of L-lysine) can be used to generate more stable peptides. Cysteine residues can be used to cyclize or attach two or more peptides together. This can be beneficial to constrain peptides into particular conformations. (Rizo and Gierasch Ann. Rev. Biochem. 61:387 (1992), incorporated herein by reference).

9. Pharmaceutical Carriers/Delivery of Pharmaceutical Products

As described above, the compositions can also be administered in vivo in a pharmaceutically acceptable carrier. By “pharmaceutically acceptable” is meant a material that is not biologically or otherwise undesirable, i.e., the material can be administered to a subject, along with the nucleic acid or vector, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

The compositions can be administered orally, parenterally (e.g., intravenously), by intramuscular injection, by intraperitoneal injection, transdermally, extracorporeally, topically or the like, including topical intranasal administration or administration by inhalant. As used herein, “topical intranasal administration” means delivery of the compositions into the nose and nasal passages through one or both of the nares and can comprise delivery by a spraying mechanism or droplet mechanism, or through aerosolization of the nucleic acid or vector. Administration of the compositions by inhalant can be through the nose or mouth via delivery by a spraying or droplet mechanism. Delivery can also be directly to any area of the respiratory system (e.g., lungs) via intubation. The exact amount of the compositions required will vary from subject to subject, depending on the species, age, weight and general condition of the subject, the severity of the allergic disorder being treated, the particular nucleic acid or vector used, its mode of administration and the like. Thus, it is not possible to specify an exact amount for every composition. However, an appropriate amount can be determined by one of ordinary skill in the art using only routine experimentation given the teachings herein.

Parenteral administration of the composition, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein.

The materials can be in solution, suspension (for example, incorporated into microparticles, liposomes, or cells). These can be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K. D., Br. J. Cancer, 60:275-281, (1989); Bagshawe, et al., Br. J. Cancer, 5.8:700-703, (1988); Senter, et al., Bioconjugate Chem., 4:3-9, (1993); Battelli, et al., Cancer Immunol. Immunother., 35:421-425, (1992); Pietersz and McKenzie, Immunolog. Reviews, 129:57-80, (1992); and Roffler, et al., Biochem. Pharmacol, 42:2062-2065, (1991)). Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399-409 (1991)).

a) Pharmaceutically Acceptable Carriers

The compositions, including antibodies, can be used therapeutically in combination with a pharmaceutically acceptable carrier.

Suitable carriers and their formulations are described in Remington: The Science and Practice of Pharmacy (19th ed.) ed. A. R. Gennaro, Mack Publishing Company, Easton, Pa. 1995. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8, and more preferably from about 7 to about 7.5. Further carriers include sustained release preparations such as semipermeable matrices of solid hydrophobic polymers containing the antibody, which matrices are in the form of shaped articles, e.g., films, liposomes or microparticles. It will be apparent to those persons skilled in the art that certain carriers can be more preferable depending upon, for instance, the route of administration and concentration of composition being administered.

Pharmaceutical carriers are known to those skilled in the art. These most typically would be standard carriers for administration of drugs to humans, including solutions such as sterile water, saline, and buffered solutions at physiological pH. The compositions can be administered intramuscularly or subcutaneously. Other compounds will be administered according to standard procedures used by those skilled in the art.

Pharmaceutical compositions can include carriers, thickeners, diluents, buffers, preservatives, surface active agents and the like in addition to the molecule of choice. Pharmaceutical compositions can also include one or more active ingredients such as antimicrobial agents, antiinflammatory agents, anesthetics, and the like.

The pharmaceutical composition can be administered in a number of ways depending on whether local or systemic treatment is desired, and on the area to be treated. Administration can be topically (including ophthalmically, vaginally, rectally, intranasally), orally, by inhalation, or parenterally, for example by intravenous drip, subcutaneous, intraperitoneal or intramuscular injection. The disclosed antibodies can be administered intravenously, intraperitoneally, intramuscularly, subcutaneously, intracavity, or transdermally.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives can also be present such as, for example, antimicrobials, anti-oxidants, chelating agents, and inert gases and the like.

Formulations for topical administration can include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids or binders may be desirable.

Some of the compositions can potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

10. Chips and Micro Arrays

Disclosed are chips where at least one address is the sequences or part of the sequences set forth in any of the nucleic acid sequences disclosed herein. Also disclosed are chips where at least one address is the sequences or portion of sequences set forth in any of the peptide sequences disclosed herein.

Also disclosed are chips where at least one address is a variant of the sequences or part of the sequences set forth in any of the nucleic acid sequences disclosed herein. Also disclosed are chips where at least one address is a variant of the sequences or portion of sequences set forth in any of the peptide sequences disclosed herein.

11. Computer Readable Mediums

It is understood that the disclosed nucleic acids and proteins can be represented as a sequence consisting of the nucleotides of amino acids. There are a variety of ways to display these sequences, for example the nucleotide guanosine can be represented by G or g. Likewise the amino acid valine can be represented by Val or V. Those of skill in the art understand how to display and express any nucleic acid or protein sequence in any of the variety of ways that exist, each of which is considered herein disclosed. Specifically contemplated herein is the display of these sequences on computer readable mediums, such as, commercially available floppy disks, tapes, chips, hard drives, compact disks, and video disks, or other computer readable mediums. Also disclosed are the binary code representations of the disclosed sequences. Those of skill in the art understand what computer readable mediums. Thus, computer readable mediums on which the nucleic acids or protein sequences are recorded, stored, or saved.

Disclosed are computer readable mediums comprising the sequences and information regarding the sequences set forth herein.

12. Kits

Disclosed herein are kits that are drawn to reagents that can be used in practicing the methods disclosed herein. The kits can include any reagent or combination of reagent discussed herein or that would be understood to be required or beneficial in the practice of the disclosed methods. For example, the kits could include primers to perform the amplification reactions discussed in certain embodiments of the methods, as well as the buffers and enzymes required to use the primers as intended.

### D. METHODS OF MAKING THE COMPOSITIONS

The compositions disclosed herein and the compositions necessary to perform the disclosed methods can be made using any method known to those of skill in the art for that particular reagent or compound unless otherwise specifically noted.

The disclosed viral vectors can be made using standard recombinant molecular biology techniques. Many of these techniques are illustrated in Maniatis (Maniatis et al., “Molecular Cloning—A Laboratory Manual,” (Cold Spring Harbor Laboratory, Latest edition) and Sambrook et al., Molecular Cloning: A Laboratory Manual, 2nd Ed., Cold Spring Harbor Laboratory, Cold Spring Harbor, N.Y., 1989.

1. Nucleic Acid Synthesis

For example, the nucleic acids, such as, the oligonucleotides to be used as primers can be made using standard chemical synthesis methods or can be produced using enzymatic methods or any other known method. Such methods can range from standard enzymatic digestion followed by nucleotide fragment isolation (see for example, Sambrook et al., Molecular Cloning: A Laboratory Manual, 2nd Edition (Cold Spring Harbor Laboratory Press, Cold Spring Harbor, N.Y., 1989) Chapters 5, 6) to purely synthetic methods, for example, by the cyanoethyl phosphoramidite method using a Milligen or Beckman System Plus DNA synthesizer (for example, Model 8700 automated synthesizer of Milligen-Biosearch, Burlington, Mass. or ABI Model 380B). Synthetic methods useful for making oligonucleotides are also described by Ikuta et al., Ann. Rev. Biochem. 53:323-356 (1984), (phosphotriester and phosphite-triester methods), and Narang et al., Methods Enzymol., 65:610-620 (1980), (phosphotriester method). Protein nucleic acid molecules can be made using known methods such as those described by Nielsen et al., Bioconjug. Chem. 5:3-7 (1994).

2. Peptide Synthesis

One method of producing the disclosed proteins is to link two or more peptides or polypeptides together by protein chemistry techniques. For example, peptides or polypeptides can be chemically synthesized using currently available laboratory equipment using either Fmoc (9-fluorenylmethyloxycarbonyl) or Boc (tert-butyloxycarbonoyl) chemistry. (Applied Biosystems, Inc., Foster City, Calif.). One skilled in the art can readily appreciate that a peptide or polypeptide corresponding to the disclosed proteins, for example, can be synthesized by standard chemical reactions. For example, a peptide or polypeptide can be synthesized and not cleaved from its synthesis resin whereas the other fragment of a peptide or protein can be synthesized and subsequently cleaved from the resin, thereby exposing a terminal group which is functionally blocked on the other fragment. By peptide condensation reactions, these two fragments can be covalently joined via a peptide bond at their carboxyl and amino termini, respectively, to form an antibody, or fragment thereof. (Grant G A (1992) Synthetic Peptides: A User Guide. W.H. Freeman and Co., N.Y. (1992); Bodansky M and Trost B., Ed. (1993) Principles of Peptide Synthesis. Springer-Verlag Inc., NY (which is herein incorporated by reference at least for material related to peptide synthesis). Alternatively, the peptide or polypeptide is independently synthesized in vivo as described herein. Once isolated, these independent peptides or polypeptides can be linked to form a peptide or fragment thereof via similar peptide condensation reactions.

For example, enzymatic ligation of cloned or synthetic peptide segments allow relatively short peptide fragments to be joined to produce larger peptide fragments, polypeptides or whole protein domains (Abrahmsen L et al., Biochemistry, 30:4151 (1991)). Alternatively, native chemical ligation of synthetic peptides can be utilized to synthetically construct large peptides or polypeptides from shorter peptide fragments. This method consists of a two step chemical reaction (Dawson et al. Synthesis of Proteins by Native Chemical Ligation. Science, 266:776-779 (1994)). The first step is the chemoselective reaction of an unprotected synthetic peptide—thioester with another unprotected peptide segment containing an amino-terminal Cys residue to give a thioester-linked intermediate as the initial covalent product. Without a change in the reaction conditions, this intermediate undergoes spontaneous, rapid intramolecular reaction to form a native peptide bond at the ligation site (Baggiolini M et al. (1992) FEBS Lett. 307:97-101; Clark-Lewis I et al., J. Biol. Chem., 269:16075 (1994); Clark-Lewis I et al., Biochemistry, 30:3128 (1991); Rajarathnam K et al., Biochemistry 33:6623-30 (1994)).

Alternatively, unprotected peptide segments are chemically linked where the bond formed between the peptide segments as a result of the chemical ligation is an unnatural (non-peptide) bond (Schnolzer, M et al. Science, 256:221 (1992)). This technique has been used to synthesize analogs of protein domains as well as large amounts of relatively pure proteins with full biological activity (deLisle Milton R C et al., Techniques in Protein Chemistry IV. Academic Press, New York, pp. 257-267 (1992)>.

3. Processes for Making the Compositions

Disclosed are processes for making the compositions as well as making the intermediates leading to the compositions. There are a variety of methods that can be used for making these compositions, such as synthetic chemical methods and standard molecular biology methods. It is understood that the methods of making these and the other disclosed compositions are specifically disclosed.

Disclosed are nucleic acid molecules produced by the process comprising linking in an operative way a promoter element and a μ-opioid receptor element.

Disclosed are nucleic acid molecules produced by the process comprising linking in an operative way nucleic acid molecules comprising sequences set forth in SEQ ID NO:2 and SEQ ID NO:4.

Also disclosed are nucleic acid molecules produced by the process comprising linking in an operative way nucleic acid molecules comprising sequences having 80% identity to sequences set forth in SEQ ID NO:2 and SEQ ID NO:4.

Also disclosed are nucleic acid molecules produced by the process comprising linking in an operative way nucleic acid molecules comprising sequences that hybridizes under stringent hybridization conditions to sequences set forth in SEQ ID NO:2 and SEQ ID NO:4.

Disclosed are nucleic acid molecules produced by the process comprising linking in an operative way a nucleic acid molecule comprising a sequence encoding a μ-opioid receptor peptide and a sequence controlling an expression of the sequence encoding the μ-opioid receptor peptide.

Disclosed are nucleic acid molecules produced by the process comprising linking in an operative way a nucleic acid molecule comprising a sequence encoding a μ-opioid receptor peptide wherein the μ-opioid receptor peptide has 80% identity to the peptide set forth in SEQ ID NO:1 or 3 and a sequence controlling expression of the sequences encoding the peptide.

Disclosed are nucleic acid molecules produced by the process comprising linking in an operative way a nucleic acid molecule comprising a sequence encoding a μ-opioid receptor peptide wherein the μ-opioid receptor peptide has 80% identity to the peptides set forth in SEQ ID NO:1 or 3 and, wherein any change from the sequences set forth in SEQ ID NO:1 or 3 are conservative changes and a sequence controlling expression of the sequences encoding the peptide.

Disclosed are cells produced by the process of transforming the cell with any of the disclosed nucleic acids. Disclosed are cells produced by the process of transforming the cell with any of the non-naturally occurring disclosed nucleic acids.

Disclosed are any of the disclosed peptides produced by the process of expressing any of the disclosed nucleic acids. Disclosed are any of the non-naturally occurring disclosed peptides produced by the process of expressing any of the disclosed nucleic acids. Disclosed are any of the disclosed peptides produced by the process of expressing any of the non-naturally disclosed nucleic acids.

Disclosed are animals produced by the process of transfecting a cell within the animal with any of the nucleic acid molecules disclosed herein. Disclosed are animals produced by the process of transfecting a cell within the animal any of the nucleic acid molecules disclosed herein, wherein the animal is a mammal. Also disclosed are animals produced by the process of transfecting a cell within the animal any of the nucleic acid molecules disclosed herein, wherein the mammal is mouse, rat, rabbit, cow, sheep, pig, or primate. Also disclosed are non human primates and non-human mammals.

Also disclose are animals produced by the process of adding to the animal any of the cells disclosed herein.

### E. METHODS OF USING THE COMPOSITIONS

1. Methods of Using the Compositions as Research Tools

The disclosed compositions can be used in a variety of ways as research tools. For example, the disclosed compositions, the μ-opioid receptor constructs, and other nucleic acids, such as SEQ ID NOs:2 and 4 can be used to produce organisms, such as transgenic or knockout mice, which can be used as model systems for the study of pain.

2. Methods of Gene Modification and Gene Disruption

The disclosed compositions and methods can be used for targeted gene disruption and modification in any animal that can undergo these events. Gene modification and gene disruption refer to the methods, techniques, and compositions that surround the selective removal or alteration of a gene or stretch of chromosome in an animal, such as a mammal, in a way that propagates the modification through the germ line of the mammal. In general, a cell is transformed with a vector which is designed to homologously recombine with a region of a particular chromosome contained within the cell, as for example, described herein. This homologous recombination event can produce a chromosome which has exogenous DNA introduced, for example in frame, with the surrounding DNA. This type of protocol allows for very specific mutations, such as point mutations, to be introduced into the genome contained within the cell. Methods for performing this type of homologous recombination are disclosed herein.

One of the preferred characteristics of performing homologous recombination in mammalian cells is that the cells should be able to be cultured, because the desired recombination event occurs at a low frequency.

Once the cell is produced through the methods described herein, an animal can be produced from this cell through either stem cell technology or cloning technology. For example, if the cell into which the nucleic acid was transfected was a stem cell for the organism, then this cell, after transfection and culturing, can be used to produce an organism which will contain the gene modification or disruption in germ line cells, which can then in turn be used to produce another animal that possesses the gene modification or disruption in all of its cells. In other methods for production of an animal containing the gene modification or disruption in all of its cells, cloning technologies can be used. These technologies generally take the nucleus of the transfected cell and either through fusion or replacement fuse the transfected nucleus with an oocyte which can then be manipulated to produce an animal. The advantage of procedures that use cloning instead of ES technology is that cells other than ES cells can be transfected. For example, a fibroblast cell, which is very easy to culture can be used as the cell which is transfected and has a gene modification or disruption event take place, and then cells derived from this cell can be used to clone a whole animal.

3. Therapeutic Uses

Effective dosages and schedules for administering the compositions can be determined empirically, and making such determinations is within the skill in the art. The dosage ranges for the administration of the compositions are those large enough to produce the desired effect in which the symptoms disorder are effected. The dosage should not be so large as to cause adverse side effects, such as unwanted cross-reactions, anaphylactic reactions, and the like. Generally, the dosage will vary with the age, condition, sex and extent of the disease in the patient, route of administration, or whether other drugs are included in the regimen, and can be determined by one of skill in the art. The dosage can be adjusted by the individual physician in the event of any counterindications. Dosage can vary, and can be administered in one or more dose administrations daily, for one or several days. Guidance can be found in the literature for appropriate dosages for given classes of pharmaceutical products.

Following administration of a disclosed composition, such as the disclosed constructs, for treating, inhibiting, or preventing pain, the efficacy of the therapeutic construct can be assessed in various ways well known to the skilled practitioner. For instance, one of ordinary skill in the art will understand that a composition, such as the disclosed constructs, disclosed herein is efficacious in treating pain or inhibiting or reducing the effects of pain in a subject by observing that the composition reduces the onset of the conditions associated with these diseases. Furthermore, the amount of protein or transcript produced from the constructs can be analyzed using any diagnostic method. For example, it can be measured using polymerase chain reaction assays to detect the presence of construct nucleic acid or antibody assays to detect the presence of protein produced from the construct in a sample (e.g., but not limited to, blood or other cells, such as neural cells) from a subject or patient, or it can be measured by any of the methods disclosed herein for monitoring non-human pain, and through communication for human pain.

### F. EXAMPLES

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

The following examples are put forth so as to provide those of ordinary skill in the art with a complete disclosure and description of how the compounds, compositions, articles, devices and/or methods claimed herein are made and evaluated, and are intended to be purely exemplary of the invention and are not intended to limit the scope of what the inventors regard as their invention. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### 1. Example 1

### Orofacial Pain

a) Methods and Approaches

Expression of the μ-opioid receptor in neurons involved in the central processing of orofacial pain will result in attenuation of nociception. FIV(μ-opioid receptor), a lentivirus capable of stably transducing terminally differentiated cells (neurons) with μ-opioid receptor, can be peripherally administered at orofacial sites, including the TMJ and the masseter muscle, exposed to nociceptive substances. The efficacy of the therapy can be assessed by assaying changes in muscle EMG as well as resistance to jaw opening.

(1) Construct FIV(HUMOR) and Confirm Its Ability to Transduce Cells of Neuronal Origin

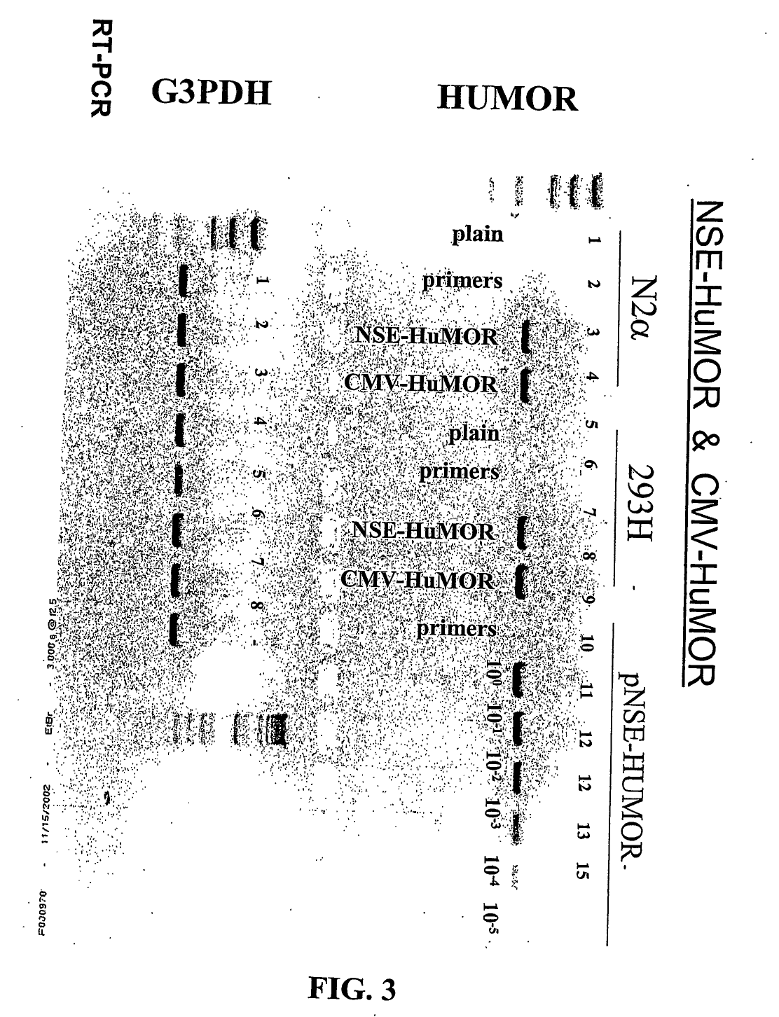

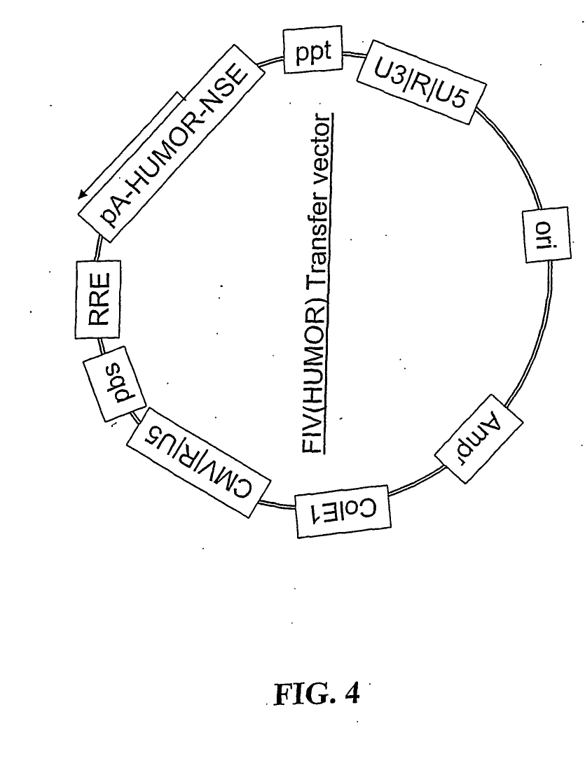

The opening reading frame of the HUMOR cDNA having the sequence set forth in SEQ ID NO:2 was (Raynor K, et al., J Pharmacol Exp Ther. 1995; 272:423-8) cloned into the pFIV vector using standard molecular biology methods generating pFIV(HUMOR) SEQ ID NO: 7 (See FIG. 4). Constructs having two different promoters were made to drive the expression of HUMOR, the neuron specific enolase (NSE) promoter as well as the cytomegalovirus (CMV) promoter. The NSE promoter limits the expression of HUMOR selectively in neurons at constitutive levels, whereas the CMV promoter results in ubiquitous levels of expression independent of cell type. Clones were selected following DNA isolation and confirmation by multiple restriction digestions and direct DNA sequencing of both strands. pFIV(HUMOR) can be co-transfected with pPAK and pVSV-G vectors in 293H cells utilizing the Lipofectamine 2000 reagent (Gibco/BRL-Invitrogen) for virus production as previously described (FIG. 3). Following 60 hours incubation, the supernatant will be collected, filtered and titered. Titers of 10⁵ ip/ml are typical using this method. If required, the titers can be increased by ultra-centrifugation of the supernatant and re-suspension in normal saline.

FIV(NSE-HUMOR) as well as FIV(CMV-HUMOR) efficacy can be tested on the N2a neuronal cell line, whereby cells can be seeded on 12 well plates and infected by FIV(HUMOR), followed by a 24 hour fresh media change. DNA, RNA and protein samples can be harvested using standard laboratory methods at 72 hours post infection. The presence of HUMOR gene copies in the infected samples can be assessed by Q-PCR, and the expression of HUMOR can be determined at the mRNA and protein levels by RT-PCR and western immunoblotting, respectively. In addition, other cells can be fixed by 4% paraformaldehyde for HUMOR protein expression detection and visualization by immunocytochemistry. FIG. 1 or 2 demonstrates the expression of HUMOR in N2a cells following transient transfection and detection of its expression by immunocytochemistry utilizing a commercially available antibody raised against HUMOR (Research & Diagnostic Antibodies, Benicia Calif. Cat# AS-3942S). The FIV vector can infect and stably transduce sensory trigeminal neurons following local peripheral administration as demonstrated by the data in FIG. 3. At the animal behavioral level, the targeted expression of HUMOR in orofacial sensory neurons can result in attenuation of nociceptive symptoms that readily can be measured as change (decrease) in EMG activity and decrease in resistance to jaw opening.

(2) Targeted Expression of μ-Opioid Receptor in Orofacial Somatosensory Neurons Can Attenuate Orofacial Pain Following Administration of Algesic Substance

The feline immunodeficiency viral vectors FIV(NSE-HUMOR) and FIV(CMV-HUMOR) can be employed in the transduction of trigeminal sensory neurons with the μ-opioid receptor. Orofacial nociception can be quantitatively assessed by means of masticatory muscle activity, as measured by electromyography (EMG), as well as resistance to mouth opening, as measured by a digital force transducer. Table 6 summarizes the experimental conditions. In brief, FIV(NSE-HUMOR) and FIV(CMV-HUMOR) can be injected (50 μl of 10⁷ ip/ml) in the TMJ or masseter muscle of anesthetized animals, which can be returned to their cages after recovery. Five weeks post-treatment, EMG bipolar percutaneous hook electrodes can be inserted in the masseter and temporal muscles under anesthesia and an orthodontic Kobayashi hook can be bonded to the mandibular incisors for attachment to the digital force gauge. The animals can then be positioned in a custom made restraining device. Base line EMG and resistance to jaw opening measurements can be taken for every animal. Four channels of simultaneous EMG signal (right and left masseter and temporal muscles) and one channel for the digital force gauge can be recorded. An A/D conversion card (NIO16E1, National Instruments) for the EMG signal and a digital force gauge (FGF series, Kemco Instruments) can be used for signal collection. A “blinded” observer can collect 10 seconds of EMG and digital force measurements at 5, 10 and 15 mm of vertical opening five times simultaneously. Data can be stored for later analysis. The appropriate animals can then be injected in the temporomandibular joint and masseter muscle area with the algesic agent glutamate (2.5 μmol in saline) or vehicle solution (saline), and EMG and resistance to jaw opening recordings can be taken. Data can be analyzed by two-way ANOVA with repeated measures. Student's t-test can compare the two groups. These experiments replicate conditions seen in the human condition. (Molin C., Acta Odontol Scand 1972; 30:485-99; Moller U M, et al., Scand J Dent Res 1984; 92: 64-83; Stohler C S, et al., Helv Odontol Acta 1985; 29:13-20; Stohler C S, et al., Arch Oral Biol 1988; 33: 175-82; Stohler C S. Temporomandibular disorders and related pain conditions. In: Sessle, B. J., Bryant, P. S. and Dionne, R. A. Editors, 1995. Progress in pain research and management Elsevier, Amsterdam, pp. 3-30; Lund J P, Stohler C S. Effect of pain on muscular activity in temporomandibular disorders and related conditions. In: Stohler, C. S., Carlson, D. S., editors. Biological and Psychological aspects of orofacial pain, craniomandibular growth series. Ann Arbor, Mich.: University of Michigan, 1994. pp. 75-91; Hoshio T. Senior Thesis, Division of Orthodontics, Eastman Department of Dentistry, University of Rochester School of Medicine and Dentistry. Rochester, N.Y.; Balkhi K M, et al., J Orofacial Pain 1993; 7:89-97).

HUMOR expression can be confirmed following the experimental recordings in deeply anesthetized animals that can be sacrificed via trans-cardial perfusion of 4% paraformaldehyde, and the brain stem, trigeminal ganglia, masseter muscle and TMJ can be excised and collected for further analysis. Specifically, we can characterize the expression of HUMOR in the central and peripheral tissues utilizing immunocytochemistry as depicted by our preliminary data. Cell type specificity can be accomplished by double immunofluorescence, as previously demonstrated by our laboratory. Potential deleterious effects of HUMOR expression (or over-expression in the CMV driven gene) to neurons or other cells can be examined by Hoechst staining and confirmed by TUNEL (in the case of cell death) as well as by the expression of neuron specific housekeeping genes, such as neuron specific enolase as well substance P (expressed selectively by sensory neurons).

TABLE 6
G. Summary of experimental conditions

| ALGESIC | SITE of | | |
|---|---|---|---|
| AGENT | NOCICEPTION | TREATMENT | N |
| Glutamate | Temporoman- | FIV(NSE-HUMOR) | 10 each |
| saline | dibular Joint | | |
| | Masseter | FIV(CMV-HUMOR) morphine | |
| | muscle | FIV(NSE-HUMOR) + opioid | |
| | | FIV(CMV-HUMOR) + opioid | |
| | | saline | |

Patients presenting with orofacial pain from TMJ disorders have been characterized as having decreased bite and chewing forces and limited jaw opening. For example, reduced grip strength (Norsdenskiold U M and Grimby G., Scand J Rheumatol 1993; 22: 14-9.), sustained jaw closing pain (Jow R W and Clark G T., Arch Oral Biol 1989; 34: 857-62.) and reduced bite strength (Molin C., Acta Odontol Scand 1972; 30:485-99) have all been reported in patients with muscle pain. The reduction in muscle force exertion associated with myalgia has been suggested to be due to reduced activity of agonist muscles and increased activity of antagonist muscles (Molin C., Acta Odontol Scand 1972; 30:485-99; Stohler C S, et al., Helv Odontol Acta 1985; 29:13-20; Stohler C S, et al., Arch Oral Biol 1988; 33: 175-82; Stohler C S. Temporomandibular disorders and related pain conditions. In: Sessle, B. J., Bryant, P. S. and Dionne, R. A. Editors, 1995. Progress in pain research and management Elsevier, Amsterdam, pp. 3-30; and Lund J P, Stohler C S. Effect of pain on muscular activity in temporomandibular disorders and related conditions. In: Stohler, C. S., Carlson, D. S., editors. Biological and Psychological aspects of orofacial pain, craniomandibular growth series. Ann Arbor, Mich.: University of Michigan, 1994. pp. 75-91.) Keil et al., (Keil L J, et al., Pain 2000; 85:333-43) have demonstrated that forelimb grip force reduction is a behavioral index of hyperalgesia in the carrageenan model of muscle hyperalgesia. This would translate to reduction of bite force and an increase in antagonist muscle activity in the orofacial region. These experiments in have been done in humans (Lund J P, Stohler C S. Effect of pain on muscular activity in temporomandibular disorders and related conditions. In: Stohler, C. S., Carlson, D. S., editors. Biological and Psychological aspects of orofacial pain, craniomandibular growth series. Ann Arbor, Mich.: University of Michigan, 1994. pp. 75-91; Hoshio T. Senior Thesis, Division of Orthodontics, Eastman Department of Dentistry, University of Rochester School of Medicine and Dentistry. Rochester, N.Y.; and Balkhi K M, et al., J Orofacial Pain 1993; 7:89-97.). Hoshio has also demonstrated that compared to controls, patients with TMD demonstrate decreased bite (201 and 223 mV for the masseter muscles respectively in asymptomatic volunteers and 128 and 153 mV for symptomatic patients). Balkhi (Balkhi K M, et al., J Orofacial Pain 1993; 7:89-97) demonstrated that chewing force was decreased in patients with pain (113 and 102 mV for deliberate right and left side chewing of gum masseter muscles respectively in asymptomatic volunteers and 85 and 83 mV for symptomatic patients). It has been demonstrated that there was increased jaw muscle activity of antagonists during jaw opening. These data clearly demonstrate that patients with TMD have similar characteristics and that decreased bite force and chewing activity are a reflection of somatic pain.

Mice (C57/B6) can be employed to make the transgenic mice.

a) Injection of Replication Defective FIV(HUMOR) Vectors

The animals, such as pups, can be anesthetized with ketamine (60 mg/Kg) and xylazine (5 mg/Kg) IP. To verify the induction of surgical anesthesia, a toe is pinched in order to test for reflex withdrawal. One type of injection will be performed in two distinct areas. Under surgical plane of anesthesia the animals, such as mice, can be injected with 50 μl of 107 ip/ml of FIV(HUMOR) using a 1 ml syringe with a 27^{1/2} gage needle directly into the temporomandibular joint and masseter muscle. The animals can be identified by ear punching. Animals can be held at the base of the tail with distal portion of tail situated on surface of nestlet, for example. Using a straight edge blade, ˜7 mm of distal tail can be removed, and the mouse can be placed in a cage and the tail specimen stored in a vial labeled by mouse ID# and sex. The mice are euthanised with sodium pentobarbital (200 mg/kg).

Fixation by intracardial transfusion can be performed. Upon exposure of the heart, the right atrium can be clipped and the left ventricle can be catheterized with a 17 gage needle through which 50 ml of 4% paraformaldehyde solution in phosphate buffered saline can be transfused into the animal. The liver, spleen, kidney and brain can be dissected and post-fixed until sectioned for histology. The middle part of the cranium, including the cranial base (sphenoid, ethmoid, maxilla) can also be dissected, demineralized by immersion into an EDTA solution and section for histology.

### 2. Example 2

### Non-Primate Lentiviral Vector Administration in the TMJ

Diclosed herein are the effects lentiviral vectors on the temporomandibular joint. Defective feline immunodeficiency virus capable of infecting dividing as well as terminally differentiated cells with the reporter gene lacZ, the expression of which was studied by means of PCR, X-gal histochemistry and β-galactosidase immunocytochemistry were injected into the articular joint space. The results showed successful transduction of hard and soft tissues of the temporomandibular joint. Interestingly, a subset of primary sensory neurons of the ipsilateral trigeminal ganglion also stained positive for the reporter gene, presumambly following uptake of the lentiviral vector by peripheral nerve fibers and retrograde transport to the nucleus. These findings indicate that transfer of anti-nociceptive genes, and disclosed herein, genes such as the opiod receptors, can be transferred into nerve cells and relieve pain. For example, lentiviral vectors can serve as the platform for the transfer of anti-nociceptive genes for the management of temporomandibular joint pain.

a) Materials and Methods

(1) Vector Construction and Packaging

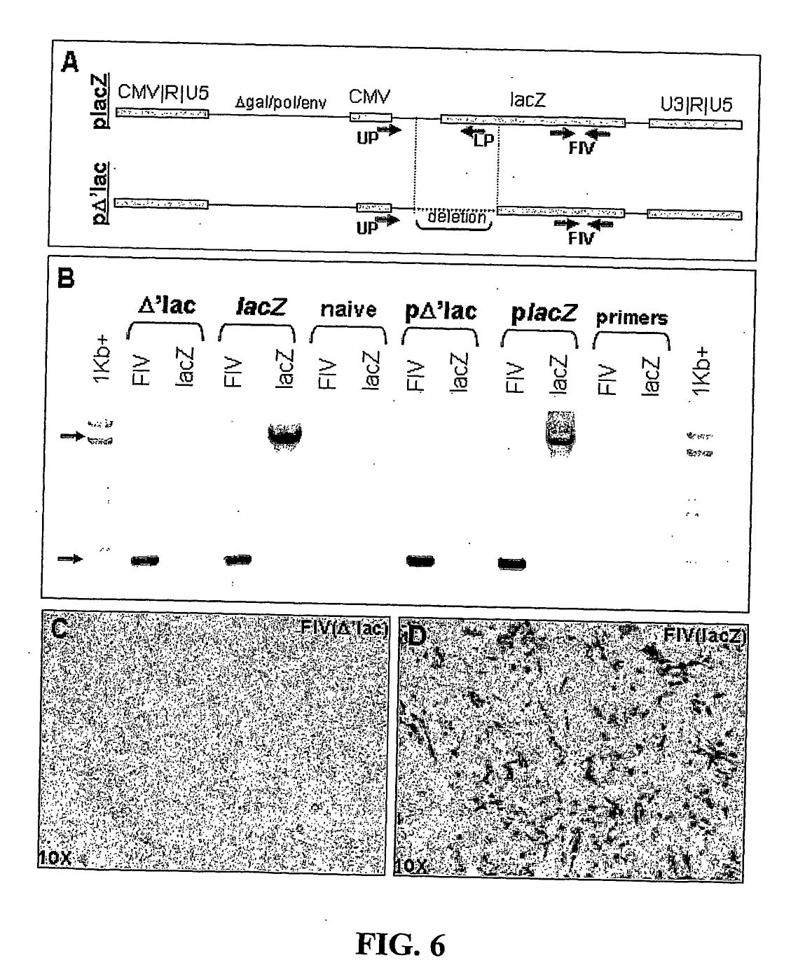

The defective, vesicular stomatitis (VSV-G) pseudotyped, feline immunodeficiency virus, FIV(lacZ), capable of transducing dividing, growth-arrested as well as post-mitotic cells (neurons) with the reporter gene lacZ driven by the ubiquitous cytomegalovirus promoter, CMV (Poeschla E M, et al. (1998). Nature Med 4: 354-357) was employed. The vectors were kindly donated to us by Dr. Wong-Staal, University of California at San Diego. A schematic description of the vector is depicted in FIG. 6A. In addition, a control FIV(Δ'lac) vector carrying an inactive β-galactosidase was constructed by deleting the first 1,000 bp of the lacZ gene (3.75 kb in total), including the transcription initiation site (FIG. 6A). Specifically, the FIV(lacZ) vector was digested in vitro with the SstII and Cla I restriction enzymes overnight at 37° C., followed by agarose gel purification. The ends of the backbone DNA were blunted with the T4 DNA polymerase (Invitrogen, Carlsbad Calif.) and ligated with T4 ligase (Invitrogen) according to manufacturer's instructions. The FIV(lacZ) and FIV(Δ'lac) vectors were transiently co-transfected along with the packaging and VSV-G vectors into 293H cells (GIBCO/BRL) cultured in DMEM (Invitrogen) plus 10% FBS (Gemini, Woodland Calif.) using the Lipofectamine 2000 reagent per manufacturer's instructions (Invitrogen), and followed by a fresh media change supplemented by non-essential amino acids (Invitrogen). Sixty hours post-transfection, the supernatant was collected, filtered through 0.45 mm Surfil®-MF filter (Corning Seperations Division, Acton Mass.), aliquoted and frozen until further use. Titering was performed on CrfK cells (American Tissue Culture Collection; Manassas, Va.) cultured in 24 well tissue culture plates, and assessed at at 5×10⁷ blue forming units (bfu)/mL by X-gal histochemistry.

(2) Animal Injections

All methods pertinent to animal utilization were approved by the University Committee on Animal Resources. Specifically, 12 male mice, C57BL/6J, under surgical plane of anesthesia (ketamine 60 mg/Kg and xylazine 5 mg/Kg administered intraperitoneally) received a single injection of 5×10⁶ FIV(lacZ) infectious particles (100 μl of stock solution) in the joint space of the right TMJ. Four additional mice received a single injection of 5×10⁶ FIV(Δ'lac) infectious particles (100 μl of stock solution) in the joint space of the right TMJ. In brief, the hair of the skin covering the right TMJ was shaved and the skin cleaned with Betadine solution. The joint was approached with an antero-posterior incision between the posterior end of the zygomatic arch and the ear cartilage, followed by a blunt dissection to expose the zygomatic arch and the posterior margin of the articular emminence. The joint space was not exposed during this procedure. The posterior margin of the emminense was identified by palpation and a 1-ml tuberculin syringe with a 27½ gage needle was employed to inject the experimental solutions in the joint. This surgically assisted intra-articular injection technique was utilized to minimize leakage or spreading of the injectable solution beyond the articular space. (Kyrkanides S, et al. (2002). J Orofac Pain 16: 229-235). In addition, 2 mice that received 100 μl saline injection served as controls. Forty-five days following treatment, the mice were deeply anesthetized by pentobarbital (100 mg/Kg IP) and euthanised by transcardial perfusion of 4% paraformaldehyde in phosphate buffered saline (PBS) (Kyrkanides S, et al. (2002a). J Orofac Pain 16: 229-235, Kyrkanides S, et al. (2002). Mol Brain Res 104: 159-169). The trigeminal ganglia and brain stem were dissected and sectioned at 20 μm using a freezing microtome. The TMJ joints were also dissected, decalcified in an EDTA buffered solution, embedded in paraffin and cut at 8 μm sections. All tissues were stored at −20° C. until further processed.

(3) X-Gal Histochemistry

Sections of trigeminal ganglia were processed by X-gal histochemistry and evaluated under light microscopy. Specifically, the sections were washed in 0.15M phosphate buffered saline (PBS) pH 7.2 for 60 min, followed by overnight processing in a staining solution containing 5-bromo-4chloro-3-indolyl-β-D-galactopyranoside (1 mg/ml), potasium ferricyanide (3 mM), potasium ferrocyanide (3 mM), NP-40 (0.02%) in 0.1M PBS pH 7.2 (Invitrogen) and MgCl₂ (1.3 mM). The tissue was then washed in PBS for 30 min, and briefly rinsed with dH₂O. Considerable attention was given so that only the bacterial form of β-galactosidase was detected. The slides were cover slipped with DPX mounting medium (Fluka, Neu-Ulm, Switzerland) and examined under a light microscope (BX51 Olympus; Tokyo, Japan). Color microphotographic images were captured in TIFF 16-bit format using a SPOT RT Color CCD digital camera attached onto the microscope and connected to a PC computer.

(4) Cell Counting

The mouse ganglia (15 mm×2 mm×3 mm) were sectioned sagitally on a freezing cryotome along their long axis into 20 μm thick sections. A total of 42 sections were approximately produced from each ganglion, which were sequentially collected onto 3 glass slides, whereby each slide contained representative ganglion sections 60 μm apart of each other. One glass slide of each ganglion was processed by X-gal histochemistry and whas employed in cell counting: all X-gal positive (blue) cells were counted on each tissue section on the slides. Since the tissue sections were 60 μm apart, counting all blue cells on a single slide gave a representative number of infected cells in each ganglion while avoiding overlap between sections and subsequently any “double counting”.

(5) Immunocytochemistry

Tissue sections from trigeminal ganglia were analyzed by immunocytochemistry employing a rabbit anti β-galactosidase polyclonal antibody (Chemicon INTL, Tamacula Calif.). In brief, sections were washed in BPS for 60 min followed by a 30 min blocking step in normal goat serum (4% in PBS) and overnight incubation in the primary antibody solution containing rabbit anti β-galactosidase polyclonal antibody (1:2,500), 0.5% Triton-X, 4% normal goat serum (Invitrogen), 1% bovine serum albumin (Sigma; St Louis, Mo.) in PBS. The next morning the tissue was washed in PBS for 60 min, followed by a 30 min blocking step and incubated for 90 min in the secondary antibody solution containing a goat anti-rabbit polyclonal antibody (1:2,000), Triton-X (0.5%) and normal goat serum (0.15%) in PBS. Subsequently, the tissue was washed in PBS for 30 min and incubated in a avidin-biodin complex solution (ABC kit, Vector Laboratories, Burlingame Calif.), and was then washed in 0.1M sodium acetate buffered solution (pH 7.4) for 30 min. The tissue was then reacted in a DAB (3,3′ diaminobenzidine)—Nickel solution in 0.1M sodium acetate buffered solution (pH 7.4) for 5 min, followed by a 15 min wash in PBS (Kyrkanides S, et al. (2002). J Orofac Pain 16: 229-235, Kyrkanides S, et al. (2002). Mol Brain Res 104: 159-169). The glass slides were then dehydrated through multiple ethanol solutions, cleared through xyaline and cover-slipped using DPX permanent mounting medium. The tissue sections were then studied under a light microscope and microphotographic images were captured as described above.

Tissue sections from the temporomadibular joints were first deparafinized by immersion in a series of xylines and alcohols, followed by antigen retrieval processing (95° C. heating for 15 sec in 0.1 M Tris-HCL buffer pH 8.9) and processing employing the aforementioned immunocytochemical method.

(6) Polymerase Chain Reaction (PCR)

The DNA from the left and right trigeminal ganglia of 8 mice (4 control and 4 experimental) was extracted employing the Trizol reagent (Invitrogen) according to manufacturer's instructions. The concentration of the recovered DNA ranged between 17-50 ng/μl, and was analyzed for the presence of viral DNA by PCR employing the following primer sets. Detection of FIV viral DNA (FIG. 6A): ^{5′}TTT TTC CAG TTC CGT TTA TCC (SEQ ID NO:35) and TTT ATC GCC AAT CCA CAT CT^{3′} SEQ ID NO. 36 (T_{A}=58° C.; 40 total cycles). Detection of active β-galactosidase gene (FIG. 6A): ^{5′}CCC ATA GTA ACG CCA ATA GG (SEQ ID NO:37) and AAA TGT GAG CGA GTA ACA ACC^{3′} SEQ ID NO. 38 (T_{A}=59.6° C.; 45 total cycles). Detection of genomic DNA was performed utilizing primers designed for the murine G3PDH house keeping gene: ACC ACA GTC CAT GCC ATC AC SEQ ID NO. 39 and TCC ACC ACC CTG TTG CTG TA SEQ ID NO. 40 (T_{A}=58° C.; 30 cycles). A total of 400 ng was used as DNA template in the PCR reactions. The PCR products were analyzed by agarose gel (1% w/v) electrophoresis and the images were captured utilizing a KODAK Image Analysis system (Rochester N.Y.).

b) RESULTS

(1) Intra-Articular FIV Injection Resulted in Tranduction of Hard and Soft Tissues

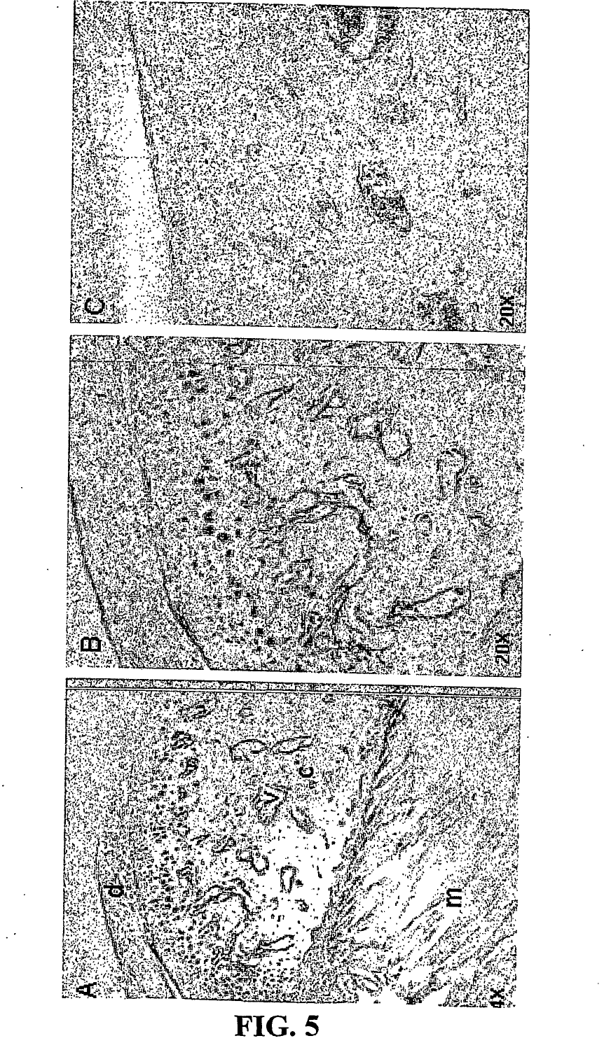

FIV(lacZ) injection into the TMJ articular space resulted in transfer of the reporter gene lacZ via the lentiviral vector in cells located within the articular capsule. Specifically, cells of the TMJ meniscus, presumably fibroblasts, expressed bacterial β-galactosidase as it was assessed by immunocytochemistry employing appropriate polyclonal antibodies. In addition, cells located in the hypertrophic zone of the condyle, primarily comprised of cartilaginous cells, as well as perivascular cells, including endothelial cells and possibly osteocytes, also stained positive for bacterial β-galactosidase (FIG. 5). There was lack of β-galactosidase in the contralateral joints as well as the saline injected animals. These results indicate FIV successfully infected and stably transferred the reporter gene to cells of hard and soft TMJ tissues.

(2) FIV Injection into the TMJ Resulted in Transduction of Trigeminal Neurons

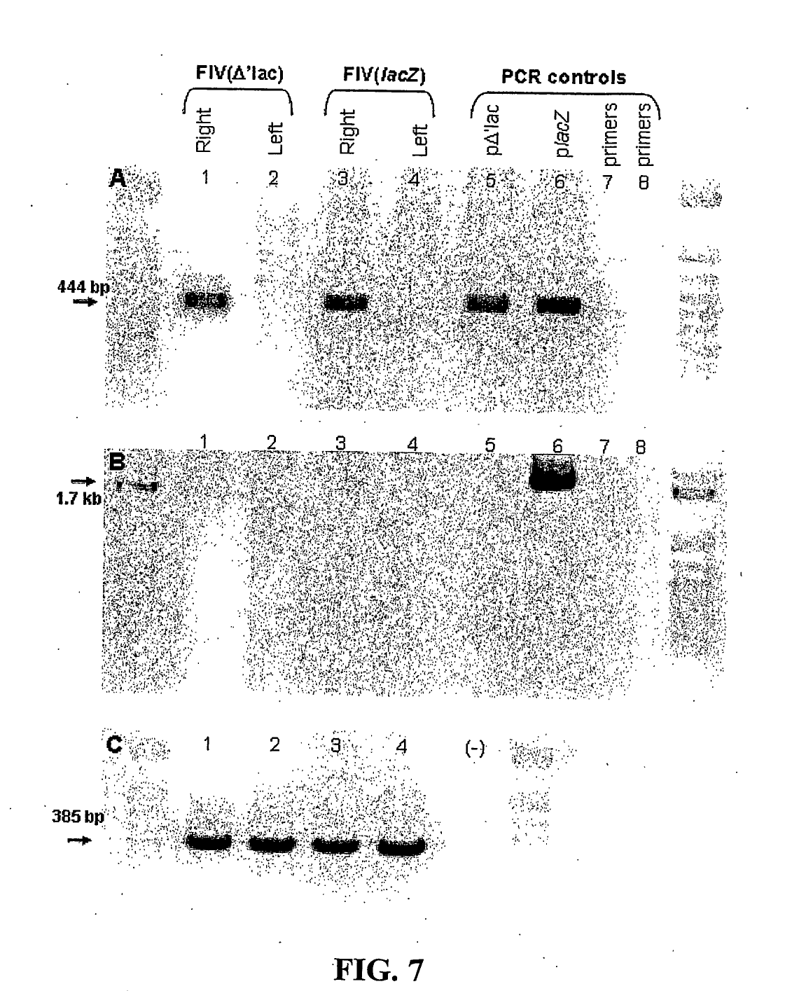

Two FIV vectors were employed in our experiment: the wild type FIV(lacZ) and the mutated FIV(Δ'lac) (FIG. 6A). FIV(Δ'lac) is capable of transducing cells with an inactive form of the reporter gene β-galactosidase compared to FIV(lacZ) which carries a full-length lacZ (FIGS. 6B & 6C). Injection of either FIV vector in the right TMJ of mice resulted in transduction of neurons located in the ipsilateral trigeminal ganglia as assessed by PCR (FIG. 7A). Full length lacZ gene was detected by PCR only in the FIV(lacZ) treated animals (FIG. 7B), accompanied by neuronal β-galactosidase expression as assessed by X-gal histochemistry. The X-gal staining was localized primarily in the posterolateral part of the ganglion within the cell bodies of cells that appear histologically as neurons (FIGS. 8A & 8B). In fact, the cell bodies of the primary sensory neurons that innervate the TMJ are known to localize in this part of the trigeminal ganglion. In contrast, FIV(Δ'lac) injected mice did not display any X-gal positive cells in the ganglia (FIG. 8C). Expression of bacterial β-galactosidase in the trigeminal ganglia was also confirmed by immunocytochemistry in the FIV(lacZ) (FIG. 8D) but not the FIV(Δ'lac) treated mice (FIG. 8E). Moreover, analysis of sections from the brain stem did not reveal any X-gal positive staining, as it was anticipated since the vectors are defective, do not replicate and cannot infect second order neurons.

(3) Considerable Number of Trigeminal Neurons were Infected by FIV(lacZ)

The mouse ganglia were on average of the following dimensions: 1.5 mm×2 mm×3 mm. As described above, a total of 42 sections (20 μm thick) were produced approximately from each ganglion, which were sequentially collected onto 3 glass slides, whereby each slide contained representative ganglion sections 60 μm apart of each other. Consistently, 4 sections were identified containing X-gal (blue) cells on each glass slide, with an average of 93 (+/−7.64 S.D.) blue cells per section. Therefore, we infer that there were approximately 93 cells×4 sections×3 glass slides=1,116 transduced neurons in each right-sided ganglion in the FIV-injected animals. No X-gal positive cells were identified in the saline injected animals. These results suggest that from a total of 5×10⁶ infectious particles injected into the articular TMJ space approximately 10³ nerve fibers were infected resulting in lacZ expression, presumambly following uptake of the lentiviral vector by peripheral nerve fibers and retrograde transport to the nucleus.

The results shown herein demonstrated that intra-articular injection of FIV(lacZ) resulted in successful gene transfer to articular TMJ surfaces as well as the joint meniscus. Interestingly, VSV-G does not require interaction between the viral envelope protein and a specific membrane receptor, but instead interacts with a phospholipid component of the cell membrane leading to membrane-fusion mediated entry. This characteristic confers broad host-cell range for VSV-G pseudotyped viruses (Burns J C, et al. (1993). Proc Natl Acad Sci USA 90: 8033-8037; Carneiro F A, et al. (2002). J Virol 76: 3756-3764). Therefore, it is possible that FIV vectors demonstrate higher infectivity for TMJ tissues than previously described viral vectors (Kuboki T, et al. (1999). Arc Oral Biol 44: 701-709), as well as result in prolonged transgene expression secondary to stable transgene integration (Poeschla E M, et al. (1998). Nature Med 4: 354-357).

The efficacy of VSV-G pseudotyped FIV vectors to transduce peripheral tissues (Kang Y, et al. (2002). J Virol 76: 9378-9388), as well as the brain (Bloemer U, et al. (1997). J Virol 71: 6641-6649) and cerebellum (Alisky J M, et al. (2002) Mol Neurosci 11: 2669-2673) has been previously demonstrated. The present observations of cells staining positively for X-gal in the trigeminal ganglion ipsilateral to the site of injection indicates that FIV virions were taken up by peripheral nerve projections of trigeminal sensory neurons that lead to infection and expression of the reporter gene lacZ by these neurons. Therefore, VSV-G pseudotyped lentiviruses, such as the defective feline or human immunodeficiency virus, can serve as the platform for the transfer of anti-nociceptive genes to temporomandibular joint tissues as well as the neurons that innervate these structures.

### 3. Example 3

a) Vector Construction

The ViraPower™ Lentiviral Expression System that can create a replication incompetent HIV-1-based lentivirus (Invitrogen, Carlsbad Calif.) was employed. This system can deliver and express NSE/Human-μ-opioid receptor in either dividing or non-dividing mammalian cells. First the pLenti6/V5-D-TOPO vector was reconstructed by insert PCR product which was generated base on the multiple cloning site of pIRES vector (Clontech Inc, Palo Alto Calif.) with the PCR primers: MCS-upper primer 5′-CACCTAATACGACTCACTATAGG-3′ SEQ ID NO. 41 and MCS-lower primer 5′-CATTAACCCTCACTAAAG-3′ SEQ ID NO. 42. This 707 bp PCR product was purified and cloned into plenti6/V5-D-TOPO vector directional (CACC, 4 base pair with overhang sequence will anneal to the GTGG sequence in the pLenti6/V5-D-TOPO vector). Multiple cloning sites were used as the template for PCR amplification to insert NheI site to the 5′ end and a multiple enzyme digestion sequence followed by a NotI site to the 3′ end of fragment. Then the CMV promoter of pLenti6/V5-D-TOPO was removed with ClaI and SpeI restriction enzyme digestions, followed by isopropanol DNA purification. Both ends of the re-constructed vector were blunted with T4 DNA polymerase (Invitrogen, Carlsbad Calif.) and ligated with T4 DNA ligase (Invitrogen) according to manufacturer's instruction.

The NSE promoter was originally from pTR-NT3myc-NSE vector (Described in Peel A L. et al., Gene Therapy. 4(1):16-24, 1997). The NSE promoter sequence can be found in SEQ ID NO:52.). The NSE fragment was cut out with BgII and HindIII restriction enzyme digestions. The BgII site of this fragment was blunted. Later on, NSE fragment (2050 bp) was ligated into HindIII and blunted XhoI sites of pBluescript II KS+/− phagemid to form pBluescript II KS-NSE.

Human-μ-opioid receptor (HUMOR) DNA was from pcDNA3-Human-μ-opioid receptor (Wang J B, et al., PNAS USA 90:10230-4) The sequence of this particular Human-μ-opioid receptor is found in SEQ ID NO:53.

This 1.6 Kb fragment was cut out of the vector with EcoRV and XbaI, and inserted into EcoRV/XbaI sites of pBluescript II KS-NSE to form pBluescript II KS-NSE-HUMOR. This structure was digested with KpnI and blunted, followed by the digestion of XbaI. The whole NSE-Human μ-opioid fragment was ligated into pIRES plasmid at XbaI and EcoRI (blunt) sites, and become pIRES-NSE-Human μ-opioid vector. To Insert the NSE-human μ-opioid receptor genes into the constructed pLenti6/V5-D-TOPO without CMV promoter, pIRES-NSE-human μ-opioid was digested with Nhe1 and Sal1 restriction enzymes and ligated into Nhe1/Sal1 sites of pLenti6N5-d-TOPO at 14° C. overnight and pLenti6/V5-D-TOPO-NSE-HUMOR vector was constructed.

In order to increase the efficacy of virus packaging, cPPT sequence was added to the front of NSE of the pLenti6/V5-D-TOPO-NSE-HUMOR. Plasmid pLP1 (SEQ ID NO:49) was used as template to PCR amplifying cPPT fragements with ClaI and NheI sites at both ends (upper primer 5′-atatcgatatcgctagcttttaaaagaaaaggggg-3′ SEQ ID NO. 43 and lower primer 5′-taatcgatgctaagcaaaattttgaatttttgtaatttg-3′ SEQ ID NO. 44). The PCR products were digested with Nhe1, and resulting fragment was ligated into the Nhe1 site of pLenti6N5-D-TOPO-NSE-HUMOR to generate pLenti6/V5-D-TOPO-cPPT-NSE-HUMOR plasmid at 4° C. overnight A schematic description of the vector is disclosed herein.

The pLenti6/v5-D-TOPO-cppt-NSE-HUMOR (SEQ ID NO:48) were transiently co-transfected along with the three packaging plasmids, pLP1(SEQ ID NO:49), pLP2 (SEQ ID NO:50), and pLP/VSVG(SEQ ID NO:51), into 293FT cells (Invitrogen) cultured in DEME (Invitrogen) plus 10% FBS (Gemini, Woodland Calif.). After 24 hours, the medium was replaced with fresh medium supplemented with non-essential amino acid (Invitrogen). Seventy hours post-transfection, the supernatant was collected and filtered through 0.45 μm Acrodisc 25 mm syringe filter (Pall Corporation, Gelman Laboratory). Aliquots of virus were frozen at −80° C. until further use. Tittering of the virus was performed on NIH3T3 cells cultured in 6 well tissue plates and assessed at 3×10³ colonies (bfu)/ml by blasticidin selection.

b) Infection of Albino Neurons Cells with Lenti6/NSE-Humor Virus

Neuro-2α cell was plated into 6 well plate and cultured in MEM (GIBCO/BRL) with 10% FBS. To infected lenti6/NSE-HUMOR virus, 1 ml viral solution (3×10³ ip) with 6 μg polybrene solution was added to N2α cell culture. After overnight plating, the medium was changed to regular MEM with 10% FBS. The cells were harvested after 96 hours infection.

c) RT Polymerase Chain Reaction (RT-PCR)

The total RNA of the N2α cells infected with Lenti6/NSE-HUMOR virus was extracted with Trizol reagent (Invitrogen) according to manufacture instruction. 5 μg total RNA was used to syntheses first strain DNA with SuperScrip™ First-Strand Synthesis System for RT-PCR (Invitrogen). Analysis of the presence of HUMOR gene was done by PCR, employing the following primer sets: 5′-GAATTACCTAATGGGAACATGG-3′ (SEQ ID NO:45) and 5′-GCAGACGATGAACACAGC-3′ (SEQ ID NO:46) (T_{A}=56° C., total 30 cycles). G3PDH house keeping gene was used as quantity PCR control. Detection of genomic G3PDH DNA was performed with primers 5′-ACCACAGCAATCAC-3′ (SEQ ID NO:47) and 5′-TCCACCACCCTGTTGCTGTA-3′ (SEQ ID NO:40) (T_{A}=58° C., 30 cycles). The PCR products were analyzed by agarose gel (1% w/v) electrophoresis and imaged were captured by a KODAK image analysis system (Rochester N.Y.).

### H. SEQUENCES

1. SEQ ID NO:1 Homo sapiens HUMOR, protein Genbank Accession No.
2. SEQ ID NO:2 Homo sapiens HUMOR, cDNA Genbank Accession No
3. SEQ ID NO:3 Murine HUMOR, protein Genbank Accession No
4. SEQ ID NO:4 Murine HUMOR, cDNA Genbank Accession No
5. SEQ ID NO:5: human kappa opioid receptor cDNA
6. SEQ ID NO:6 human delta opioid receptor cDNA sequence
7. SEQ ID NO:7 FIV(Opioid receptor construct)
8. SEQ ID NO:8 FIV(LacZ) (a construct can be used for nerve transduction)
9. SEQ ID NO:9 HUMOR degenerate cDNA G to A change at position 94
10. SEQ ID NO:10: HUMOR polypeptide conservative substitution of Val32 to I32
11. SEQ ID NO:11: Neuron specific enolase promoter
12. SEQ ID NO:12 FIV backbone
13. SEQ ID NO:13: Packaging vector
14. SEQ ID NO:14 FIV-NSE-HUMOR-pA
15. SEQ ID NO:15: Mu-opioid RECEPTOR Bovine ACCESSION NP_{—}776833
16. SEQ ID NO:16: Bos taurus mu opioid receptor mRNA, complete cds. ACCESSION U89677
17. SEQ ID NO:17:mu opioid receptor—mouse.
18. SEQ ID NO:18: Mus musculus mu opioid receptor cDNA, complete cds ACCESSION U19380
19. SEQ ID NO:19: mu opioid receptor—rat ACCESSION 156504
20. SEQ ID NO:20: Rat mu opioid receptor mRNA, complete cds.
21. SEQ ID NO:21: mu opioid receptor [Sus scrofa] porcine ACCESSION AAB53770
22. SEQ ID NO:22: Sus scrofa porcine mu opioid receptor mRNA, complete cds ACCESSION AF521309
23. SEQ ID NO:23: DELTA-opioid RECEPTOR ACCESSION AAA18789
24. SEQ ID NO:24: Human delta opioid receptor mRNA, complete cds ACCESSION U07882
25. SEQ ID NO:25: delta opioid receptor [Sus scrofa] ACCESSION AAB39694
26. SEQ ID NO:26: delta opioid receptor [Rattus norvegicus] ACCESSION AAA19939
27. SEQ ID NO:27: delta-oploid receptor [Mus musculus] ACCESSION AAA37522
28. SEQ ID NO:28: Mus musculus delta-opioid receptor mRNA, cpl cds Acc No. L06322
29. SEQ ID NO: 29: Homo sapiens (human) kappa opioid receptor ACCESSION AAA63906
30. SEQ ID NO: 30: Human kappa opioid receptor (hKOR) mRNA, complete cds Acc No. U17298
31. SEQ ID NO: 31: kappa opioid receptor [Mus musculus] ACCESSION AAA39363
32. SEQ ID NO: 32: Mouse kappa opioid receptor mRNA, complete cds ACCESSION L11065
33. SEQ ID NO: 33: kappa opioid receptor [Rattus norvegicus] ACCESSION AAA41496
34. SEQ ID NO: 34: Rattus norvegicus mRNA for kappa opioid receptor, complete cds ACCESSION D16829

### I. REFERENCES

Alisky J M, Hughes S M, Sauter S L, Joly D, Dubensky Jr. T W, Staber, P D, et al. (2002) Transduction of murine cerebellar neurons with recombinant FIV and AAV5 vectors. Mol Neurosci 11: 2669-2673.
Baum B J, Kok M, Tran S D, Yamano S (2002). The impact of gene therapy on dentistry: a revisiting after six years. JADA 133: 3544.
Bernick S (1962). The vascular and nerve supply to the temporomandibular joint of the rat. Oral Surg 15:488-492.
Bloemer U, Naldini L, Kafri T, Trono D, Verma I M, Gage F H (1997). Highly efficient and sustained gene transfer in adult neurons with a lentivirus vector. J Virol 71: 6641-6649.

Burns J C, Fiedmann T, Driever W, Burrascanno M, Yee J-K (1993). Vesicular stomatitis virus G glycoprotein pseudotyped retroviral vectors: Concentration to very high titer and efficient gene transfer into mammalian and nonmammalian cells. Proc Natl Acad Sci USA 90: 8033-8037.
Capra N F (1987). Localization and central projections of primary afferent neurons that innervate the temporomandibular joint in cats. Somatosensory Res 4: 201-213.
Carneiro F A, Bianconi L, Weissmueller G, Stauffer F, Da Poian A T (2002). Membrane recognition by vesicular stomatitis virus involves enthalpy-driven protein-lipid interactions. J Virol 76: 3756-3764.
Cox B M, Ginsburg M, Osman O H (1968). Acute tolerance to narcotic drugs in rats. Br J Pharmacol 245-256.
Dreessen D, Halata Z, Strasmann T (1990). Sensory innervation of the temporomandibular joint in the mouse. Acta Anat 139:154-160.
Frommer J, Monroe C W (1966). The morphology and distribution of nerve fibers and endings associated with the mandibular joint of the mouse. J Dent Res 45:1762-1766.
Kang Y, Stein C S, Heth P A, Sinn P L, Penisten A K, Staber P D, et al. (2002). In vivo gene transfer using a nonprimate lentiviral vector pseudotyped with ross river virus glycoproteins. J Virol 76: 9378-9388.
Kido M A, Kiyoshima T, Kondo T, Ayasaka N, Moroi R, Terada Y, Tanaka T (1993). Distribution of substance P and CGRP-like immunoreactive nerve fibers in the rat temporomandibular joint. J Dent Res 72:592-598.
Kido M A, Kondo T, Ayasaka N, Terada Y, Tanaka T (1991). The peripheral distribution of trigeminal nerve fibers in the rat temporomandibular joint studied by an anterograde axonal transport method with wheat germ agglutinin-horseradish peroxidase. Arch Oral Biol 36:397-400.
Klineberg I (1971). Structure and function of temporomandibular joint innervation. Ann Royal Coll Surg Engl 49:268-288
Kuboki T, Nakanishi T, Kanyama M, Sonoyama W, Fujisawa T, Kobayashi K, et al. (1999). Direct adenovirus-mediated gene delivery to the temporomandibular joint in guinea-pigs. Arc Oral Biol 44: 701-709.
Kyrkanides S, Moore A H, Olschowka J A, Daeschner J C, Williams J P, Hansen J T, O'Banion M K (2002). Cyclooxygenase-2 modulates brain inflammation-related gene expression in central nervous system radiation injery. Mol Brain Res 104: 159-169.
Kyrkanides S, Tallents R H, Macher D J, Olschowka J O, Stevens S Y (2002). Temporomandibular joint nociception: Effects of capsaicin on substance P-like immunoreactivity in the rabbit brain stem. J Orofac Pain 16: 229-235.
Martin W R, Eades C G (1961). Demonstration of tolerance and physical dependence in the dog following a short-term infusion of morphine. J Pharmacol Exp Ther 133: 262-270.
Peel A L. Zolotukhin S. Schrimsher G W. Muzyczka N. Reier P J. Efficient transduction of green fluorescent protein in spinal cord neurons using adeno-associated virus vectors containing cell type-specific promoters. [Journal Article] Gene Therapy. 4(1):16-24, 1997
Poeschla E M, Wong-Stall F, Looney Dl (1998). Efficient transduction of nondividing human cells by feline immunodeficiency virus lentiviral vectors. Nature Med 4: 354-357.
Pohl M, Braz J (2001). Gene therapy of pain: emerging strategies and future directions. Eur J Pharmacol 429: 39-48.
Romfh J H, Capra N F, Gatipon G B (1979). Trigeminal nerve and temporomandibular joint of the cat: A horseradish peroxidase study. Exp Neurol 65: 99-106.
Sessle B J, Hu J W (1991). Mechanisms of pain arising from articular tissues. Can J Physiol Pharmacol 69: 617-626.
Thilander B (1964). Innervation of the temporomandibular disc in Man. Acta Odont Scan 22:151-156.
Wang J B, Imai Y, Eppler C M, Gregor P, Spivak C E, Uhl G R. (1993). mu opiate receptor: cDNA cloning and expression. PNAS USA 90:10230-4
Wink C S, St'Onge M, Zimny M L (1992). Neural elements in the human temporomandibular articular disc. J Oral Maxillofac Surg 50:334-337
Wu C L, Garry M G, Zollo R A, Yang J (2001). Gene therapy for the management of pain: part II: molecular targets. Anesthesiology 95: 216-240.
Yoshino K, Kawagishi S, Amano N (1998). Morphological characteristics of primary sensory and post-synaptic sympathetic neurons supplying the temporomandibular joint in the cat Arc Oral Biol; 43: 679-686.

What is claimed is:

1. A vector for delivering an opioid receptor to a nerve cell, comprising sequence encoding an opioid receptor and a vector backbone.

2. The vector of claim 1, wherein the opioid receptor is a μ-opioid receptor.

3. The vector of claim 2, wherein the μ-opioid receptor has a sequence with at least 80% identity to the sequence set forth in SEQ ID NO:1.

4. The vector of claim 2, wherein the μ-opioid receptor has a sequence with at least 85% identity to the sequence set forth in SEQ ID NO:1.

5. The vector of claim 2, wherein the μ-opioid receptor has a sequence with at least 90% identity to the sequence set forth in SEQ ID NO:1.

6. The vector of claim 2, wherein the μ-opioid receptor has a sequence with at least 95% identity to the sequence set forth in SEQ ID NO:1.

7. The vector of claim 1, wherein the composition further comprises a promoter.

8. The vector of claim 7, wherein the promoter is a nerve cell specific promoter.

9. The vector of claim 8, wherein the promoter is the neuron specific enolase promoter.

10. The vector of claim 7, wherein the vector backbone is a lentiviral vector backbone.

11. The vector of claim 10, wherein the lentiviral vector backbone is a feline immunodeficiency vector (FIV).

12. The vector of claim 11, wherein the FIV has a sequence with at least 80% identity to the sequence set forth in SEQ ID NO:7.

13. A cell comprising the vector of claim 1.

14. An animal comprising the cell of claim 13.

15. A cell comprising the integrated product of the vector of claim 1.

16. An animal comprising the cell of claim 15.

17. A method of reducing pain in a subject, comprising administering the vector of claim 1, to the subject, wherein the vector transduces a nerve cell.

18. The method of claim 17, wherein administering the vector occurs at the point of pain.

19. The method of claim 17, wherein administering the vector occurs at the distal end of the nerve cell.

20. The method of claim 17, wherein administering the vector occurs in the peripheral nervous system.

21. The method of claim 17, wherein administering the vector occurs at the axon or axon terminal of the nerve cell.

22. The method of claim 17, wherein administering the vector occurs at the dendrite of the nerve cell.

23. The method of claim 17, wherein administering the vector occurs at the trigeminal ganglion.

24. The vector of claim 1, wherein the vector comprises the sequence set forth in SEQ ID NO:48.

25. The vector of claim 1, wherein the vector comprises the sequence set forth in SEQ ID NO: 7.

26. A method of producing the vector of claim 1, comprising linking the opiod receptor sequence operably to a promoter.

27. A method of producing the cell of claim 13, comprising transfecting the vector of claim 1 into the cell.

28. An animal produced by the process of administering the vector of claim 1 to the animal.

29. The vector of claim 7, wherein the vector backbone is an HIV vector backbone.

30. The vector of claim 7, wherin the promoter is a NSE promoter.
